SEQUENCE LISTING (237 sequences)

SEQ ID NO: 1 - DNA, 22 bp, artificial
  synthetic
  modified_base (3)..(3): c is methyl-cytosine
  modified_base (12)..(12): c is methyl-cytosine
  modified_base (15)..(15): c is methyl-cytosine
  modified_base (18)..(18): c is methyl-cytosine
  modified_base (21)..(21): c is methyl-cytosine
aactatacaatctactacctca

SEQ ID NO: 2 - DNA, 23 bp, artificial
  synthetic
  modified_base (2)..(2): c is methyl-cytosine
  modified_base (20)..(20): c is methyl-cytosine
tcagttttgcatggatttgcaca

SEQ ID NO: 3 - DNA, 24 bp, artificial
  synthetic
actacctgcactgtaagcactttg

SEQ ID NO: 4 - DNA, 24 bp, artificial
  synthetic
  modified_base (4)..(4): c is methyl-cytosine
  modified_base (13)..(13): c is methyl-cytosine
atccaatcagttcctgatgcagta

SEQ ID NO: 5 - DNA, 21 bp, artificial
  synthetic
acatggttagatcaagcacaa

SEQ ID NO: 6 - DNA, 24 bp, artificial
  synthetic
  modified_base (6)..(6): c is methyl-cytosine
gagacccagtagccagatgtagct

SEQ ID NO: 7 - DNA, 19 bp, artificial
  synthetic
  modified_base (3)..(3): c is methyl-cytosine
  modified_base (9)..(9): c is methyl-cytosine
  modified_base (12)..(12): c is methyl-cytosine
  modified_base (15)..(15): c is methyl-cytosine
  modified_base (18)..(18): c is methyl-cytosine
agcacaaactactacctca

SEQ ID NO: 8 - DNA, 20 bp, artificial
  synthetic
cagaacttagccactgtgaa

SEQ ID NO: 9 - DNA, 23 bp, artificial
  synthetic
  modified_base (13)..(13): c is methyl-cytosine
  modified_base (19)..(19): c is methyl-cytosine
gctttgacaatactattgcactg

SEQ ID NO: 10 - DNA, 21 bp, artificial
  synthetic
gctgagtgtaggatgtttaca

SEQ ID NO: 11 - DNA, 22 bp, artificial
  synthetic
cacaagttcggatctacgggtt

SEQ ID NO: 12 - DNA, 23 bp, artificial
  synthetic
  modified_base (3)..(3): c is methyl-cytosine
  modified_base (6)..(6): c is methyl-cytosine
  modified_base (18)..(18): c is methyl-cytosine
  modified_base (21)..(21): c is methyl-cytosine
aacaaccagctaagacactgcca

SEQ ID NO: 13 - DNA, 21 bp, artificial
  synthetic
  modified_base (16)..(16): c is methyl-cytosine
  modified_base (19)..(19): c is methyl-cytosine
aacaaaatcactagtcttcca

SEQ ID NO: 14 - DNA, 22 bp, artificial
  synthetic
  modified_base (4)..(4): c is methyl-cytosine
tcacaagttagggtctcaggga

SEQ ID NO: 15 - DNA, 22 bp, artificial
  synthetic
acagctggttgaaggggaccaa

SEQ ID NO: 16 - DNA, 22 bp, artificial
  synthetic
  modified_base (4)..(4): c is methyl-cytosine
  modified_base (13)..(13): c is methyl-cytosine
cttcagttatcacagtactgta

SEQ ID NO: 17 - DNA, 21 bp, artificial
  synthetic
  modified_base (7)..(7): c is methyl-cytosine
  modified_base (16)..(16): c is methyl-cytosine
aatgcccctaaaaatccttat

SEQ ID NO: 18 - DNA, 23 bp, artificial
  synthetic
  modified_base (8)..(8): c is methyl-cytosine
  modified_base (14)..(14): c is methyl-cytosine
tgatagccctgtacaatgctgct

SEQ ID NO: 19 - DNA, 20 bp, artificial
  synthetic
  modified_base (4)..(4): c is methyl-cytosine
  modified_base (13)..(13): c is methyl-cytosine
tcacttttgtgactatgcaa

SEQ ID NO: 20 - DNA, 22 bp, artificial
  synthetic
  modified_base (2)..(2): c is methyl-cytosine
  modified_base (8)..(8): c is methyl-cytosine
acaaattcggttctacagggta

SEQ ID NO: 21 - DNA, 22 bp, artificial
  synthetic
  modified_base (4)..(4): c is methyl-cytosine
  modified_base (10)..(10): c is methyl-cytosine
acacaaattcggttctacaggg

SEQ ID NO: 22 - DNA, 22 bp, artificial
  synthetic
tccacatggagttgctgttaca

SEQ ID NO: 23 - DNA, 23 bp, artificial
  synthetic
  modified_base (12)..(12): c is methyl-cytosine
gaacaggtagtctgaacactggg

SEQ ID NO: 24 - DNA, 22 bp, artificial
  synthetic
  modified_base (4)..(4): c is methyl-cytosine
aaccaatgtgcagactactgta

SEQ ID NO: 25 - DNA, 23 bp, artificial
  synthetic
ctacctgcactataagcacttta

SEQ ID NO: 26 - DNA, 21 bp, artificial
  synthetic
  modified_base (9)..(9): c is methyl-cytosine
  modified_base (15)..(15): c is methyl-cytosine
  modified_base (18)..(18): c is methyl-cytosine
ctgcctgtctgtgcctgctgt

SEQ ID NO: 27 - DNA, 21 bp, artificial
  synthetic
agaattgcgtttggacaatca

SEQ ID NO: 28 - DNA, 21 bp, artificial
  synthetic
  modified_base (20)..(20): c is methyl-cytosine
ggggtatttgacaaactgaca

SEQ ID NO: 29 - DNA, 21 bp, artificial
  synthetic
  modified_base (8)..(8): c is methyl-cytosine
ggaaatccctggcaatgtgat

SEQ ID NO: 30 - DNA, 22 bp, artificial
  synthetic
  modified_base (20)..(20): c is methyl-cytosine
ctgttcctgctgaactgagcca

SEQ ID NO: 31 - DNA, 22 bp, artificial
  synthetic
  modified_base (4)..(4): c is methyl-cytosine
agcctatcctggattacttgaa

SEQ ID NO: 32 - DNA, 21 bp, artificial
  synthetic
  modified_base (12)..(12): c is methyl-cytosine
gcattattactcacggtacga

SEQ ID NO: 33 - DNA, 21 bp, artificial
  synthetic
  modified_base (3)..(3): c is methyl-cytosine
cgcgtaccaaaagtaataatg

SEQ ID NO: 34 - DNA, 22 bp, artificial
  synthetic
  modified_base (9)..(9): c is methyl-cytosine
  modified_base (15)..(15): c is methyl-cytosine
aaaagagaccggttcactgtga

SEQ ID NO: 35 - DNA, 21 bp, artificial
  synthetic
  modified_base (3)..(3): c is methyl-cytosine
  modified_base (9)..(9): c is methyl-cytosine
aacaaaatcacaagtcttcca

SEQ ID NO: 36 - DNA, 22 bp, artificial
  synthetic
  modified_base (3)..(3): c is methyl-cytosine
  modified_base (12)..(12): c is methyl-cytosine
  modified_base (15)..(15): c is methyl-cytosine
  modified_base (18)..(18): c is methyl-cytosine
  modified_base (21)..(21): c is methyl-cytosine
aaccatacaacctactacctca

SEQ ID NO: 37 - DNA, 22 bp, artificial
  synthetic
  modified_base (3)..(3): c is methyl-cytosine
  modified_base (6)..(6): c is methyl-cytosine
  modified_base (12)..(12): c is methyl-cytosine
  modified_base (15)..(15): c is methyl-cytosine
  modified_base (18)..(18): c is methyl-cytosine
  modified_base (21)..(21): c is methyl-cytosine
aaccacacaacctactacctca

SEQ ID NO: 38 - DNA, 23 bp, artificial
  synthetic
  modified_base (2)..(2): c is methyl-cytosine
  modified_base (8)..(8): c is methyl-cytosine
  modified_base (14)..(14): c is methyl-cytosine
tcatagccctgtacaatgctgct

SEQ ID NO: 39 - DNA, 22 bp, artificial
  synthetic
  modified_base (7)..(7): c is methyl-cytosine
  modified_base (13)..(13): c is methyl-cytosine
  modified_base (16)..(16): c is methyl-cytosine
agcaagcccagaccgcaaaaag

SEQ ID NO: 40 - DNA, 22 bp, artificial
  synthetic
  modified_base (20)..(20): c is methyl-cytosine
atgctttttggggtaagggctt

SEQ ID NO: 41 - DNA, 20 bp, artificial
  synthetic
  modified_base (3)..(3): c is methyl-cytosine
  modified_base (18)..(18): c is methyl-cytosine
gcccttttaacattgcactg

SEQ ID NO: 42 - DNA, 22 bp, artificial
  synthetic
cgaccatggctgtagactgtta

SEQ ID NO: 43 - DNA, 23 bp, artificial
  synthetic
  modified_base (4)..(4): c is methyl-cytosine
  modified_base (19)..(19): c is methyl-cytosine
tcacataggaataaaaagccata

SEQ ID NO: 44 - DNA, 22 bp, artificial
  synthetic
ctacgcgtattcttaagcaata

SEQ ID NO: 45 - DNA, 22 bp, artificial
  synthetic
  modified_base (10)..(10): c is methyl-cytosine
acatcgttaccagacagtgtta

SEQ ID NO: 46 - DNA, 23 bp, artificial
  synthetic
  modified_base (2)..(2): c is methyl-cytosine
  modified_base (17)..(17): c is methyl-cytosine
tccataaagtaggaaacactaca

SEQ ID NO: 47 - DNA, 20 bp, artificial
  synthetic
  modified_base (7)..(7): c is methyl-cytosine
gtagtgctttctactttatg

SEQ ID NO: 48 - DNA, 24 bp, artificial
  synthetic
  modified_base (3)..(3): c is methyl-cytosine
aacccaccgacagcaatgaatgtt

SEQ ID NO: 49 - DNA, 23 bp, artificial
  synthetic
cagtgaattctaccagtgccata

SEQ ID NO: 50 - DNA, 22 bp, artificial
  synthetic
  modified_base (3)..(3): c is methyl-cytosine
  modified_base (12)..(12): c is methyl-cytosine
  modified_base (21)..(21): c is methyl-cytosine
acctaatatatcaaacatatca

SEQ ID NO: 51 - DNA, 21 bp, artificial
  synthetic
ctgggactttgtaggccagtt

SEQ ID NO: 52 - DNA, 23 bp, artificial
  synthetic
  modified_base (2)..(2): c is methyl-cytosine
  modified_base (20)..(20): c is methyl-cytosine
tcagttttgcatagatttgcaca

SEQ ID NO: 53 - DNA, 23 bp, artificial
  synthetic
caggcataggatgacaaagggaa

SEQ ID NO: 54 - DNA, 22 bp, artificial
  synthetic
cagactccggtggaatgaagga

SEQ ID NO: 55 - DNA, 21 bp, artificial
  synthetic
  modified_base (3)..(3): c is methyl-cytosine
  modified_base (9)..(9): c is methyl-cytosine
cacagttgccagctgagatta

SEQ ID NO: 56 - DNA, 23 bp, artificial
  synthetic
  modified_base (5)..(5): c is methyl-cytosine
  modified_base (14)..(14): c is methyl-cytosine
gaaacccagcagacaatgtagct

SEQ ID NO: 57 - DNA, 22 bp, artificial
  synthetic
  modified_base (7)..(7): c is methyl-cytosine
tcagaccgagacaagtgcaatg

SEQ ID NO: 58 - DNA, 22 bp, artificial
  synthetic
  modified_base (4)..(4): c is methyl-cytosine
taaccgatttcaaatggtgcta

SEQ ID NO: 59 - DNA, 22 bp, artificial
  synthetic
  modified_base (2)..(2): c is methyl-cytosine
  modified_base (11)..(11): c is methyl-cytosine
  modified_base (20)..(20): c is methyl-cytosine
acactgatttcaaatggtgcta

SEQ ID NO: 60 - DNA, 23 bp, artificial
  synthetic
  modified_base (2)..(2): c is methyl-cytosine
gctgagagtgtaggatgtttaca

SEQ ID NO: 61 - DNA, 21 bp, artificial
  synthetic
  modified_base (18)..(18): c is methyl-cytosine
ctaccatagggtaaaaccact

SEQ ID NO: 62 - DNA, 21 bp, artificial
  synthetic
  modified_base (6)..(6): c is methyl-cytosine
actttcggttatctagcttta

SEQ ID NO: 63 - DNA, 22 bp, artificial
  synthetic
  modified_base (2)..(2): c is methyl-cytosine
acaggccgggacaagtgcaata

SEQ ID NO: 64 - DNA, 23 bp, artificial
  synthetic
  modified_base (20)..(20): c is methyl-cytosine
agcaaaaatgtgctagtgccaaa

SEQ ID NO: 65 - DNA, 22 bp, artificial
  synthetic
cacaagatcggatctacgggtt

SEQ ID NO: 66 - DNA, 24 bp, artificial
  synthetic
aagggattcctgggaaaactggac

SEQ ID NO: 67 - DNA, 22 bp, artificial
  synthetic
  modified_base (3)..(3): c is methyl-cytosine
cccctatcacgattagcattaa

SEQ ID NO: 68 - DNA, 22 bp, artificial
  synthetic
  modified_base (3)..(3): c is methyl-cytosine
aaccgatttcaaatggtgctag

SEQ ID NO: 69 - DNA, 23 bp, artificial
  synthetic
  modified_base (4)..(4): c is methyl-cytosine
  modified_base (16)..(16): c is methyl-cytosine
gtccgtggttctaccctgtggta

SEQ ID NO: 70 - DNA, 22 bp, artificial
  synthetic
  modified_base (4)..(4): c is methyl-cytosine
  modified_base (16)..(16): c is methyl-cytosine
ccacacacttccttacattcca

SEQ ID NO: 71 - DNA, 22 bp, artificial
  synthetic
  modified_base (4)..(4): c is methyl-cytosine
  modified_base (10)..(10): c is methyl-cytosine
  modified_base (13)..(13): c is methyl-cytosine
tggcattcaccgcgtgccttaa

SEQ ID NO: 72 - DNA, 23 bp, artificial
  synthetic
  modified_base (6)..(6): c is methyl-cytosine
  modified_base (9)..(9): c is methyl-cytosine
  modified_base (15)..(15): c is methyl-cytosine
  modified_base (21)..(21): c is methyl-cytosine
acaaacaccattgtcacactcca

SEQ ID NO: 73 - DNA, 21 bp, artificial
  synthetic
  modified_base (3)..(3): c is methyl-cytosine
  modified_base (15)..(15): c is methyl-cytosine
tacatacttctttacattcca

SEQ ID NO: 74 - DNA, 23 bp, artificial
  synthetic
  modified_base (6)..(6): c is methyl-cytosine
actcaccgacagcgttgaatgtt

SEQ ID NO: 75 - DNA, 23 bp, artificial
  synthetic
  modified_base (14)..(14): c is methyl-cytosine
  modified_base (17)..(17): c is methyl-cytosine
cacaaattcggatctacagggta

SEQ ID NO: 76 - DNA, 21 bp, artificial
  synthetic
  modified_base (19)..(19): c is methyl-cytosine
ccaacaacatgaaactaccta

SEQ ID NO: 77 - DNA, 22 bp, artificial
  synthetic
  modified_base (3)..(3): c is methyl-cytosine
  modified_base (12)..(12): c is methyl-cytosine
  modified_base (15)..(15): c is methyl-cytosine
  modified_base (18)..(18): c is methyl-cytosine
  modified_base (21)..(21): c is methyl-cytosine
aactatacaacctactacctca

SEQ ID NO: 78 - DNA, 23 bp, artificial
  synthetic
  modified_base (6)..(6): c is methyl-cytosine
  modified_base (9)..(9): c is methyl-cytosine
  modified_base (18)..(18): c is methyl-cytosine
tcatacagctagataaccaaaga

SEQ ID NO: 79 - DNA, 20 bp, artificial
  synthetic
  modified_base (4)..(4): c is methyl-cytosine
  modified_base (10)..(10): c is methyl-cytosine
  modified_base (16)..(16): c is methyl-cytosine
agccgctgtcacacgcacag

SEQ ID NO: 80 - DNA, 21 bp, artificial
  synthetic
  modified_base (6)..(6): c is methyl-cytosine
  modified_base (9)..(9): c is methyl-cytosine
  modified_base (12)..(12): c is methyl-cytosine
tagtacatcatctatactgta

SEQ ID NO: 81 - DNA, 22 bp, artificial
  synthetic
  modified_base (12)..(12): c is methyl-cytosine
  modified_base (18)..(18): c is methyl-cytosine
caacaaaatcactgatgctgga

SEQ ID NO: 82 - DNA, 20 bp, artificial
  synthetic
  modified_base (16)..(16): c is methyl-cytosine
ggctgcaacacaagacacga

SEQ ID NO: 83 - DNA, 23 bp, artificial
  synthetic
  modified_base (14)..(14): c is methyl-cytosine
catcattaccaggcagtattaga

SEQ ID NO: 84 - DNA, 21 bp, artificial
  synthetic
  modified_base (2)..(2): c is methyl-cytosine
  modified_base (8)..(8): c is methyl-cytosine
  modified_base (20)..(20): c is methyl-cytosine
cccttatcagttctccgtcca

SEQ ID NO: 85 - DNA, 21 bp, artificial
  synthetic
  modified_base (12)..(12): c is methyl-cytosine
  modified_base (15)..(15): c is methyl-cytosine
gcggaacttagccactgtgaa

SEQ ID NO: 86 - DNA, 19 bp, artificial
  synthetic
  modified_base (10)..(10): c is methyl-cytosine
ctgtcaattcataggtcat

SEQ ID NO: 87 - DNA, 20 bp, artificial
  synthetic
  modified_base (18)..(18): c is methyl-cytosine
agtggtcctaaacatttcac

SEQ ID NO: 88 - DNA, 20 bp, artificial
  synthetic
ccaatatttacgtgctgcta

SEQ ID NO: 89 - DNA, 19 bp, artificial
  synthetic
  modified_base (14)..(14): c is methyl-cytosine
aagttctgtcatgcactga

SEQ ID NO: 90 - DNA, 22 bp, artificial
  synthetic
ctacctgcacaaacagcacttt

SEQ ID NO: 91 - DNA, 22 bp, artificial
  synthetic
  modified_base (7)..(7): c is methyl-cytosine
  modified_base (10)..(10): c is methyl-cytosine
  modified_base (13)..(13): c is methyl-cytosine
acagttcttcagctggcagctt

SEQ ID NO: 92 - DNA, 22 bp, artificial
  synthetic
  modified_base (5)..(5): c is methyl-cytosine
ggtacagtcaacggtcgatggt

SEQ ID NO: 93 - DNA, 21 bp, artificial
  synthetic
  modified_base (4)..(4): c is methyl-cytosine
  modified_base (10)..(10): c is methyl-cytosine
  modified_base (13)..(13): c is methyl-cytosine
  modified_base (16)..(16): c is methyl-cytosine
cagctatgccaacatcttgcc

SEQ ID NO: 94 - DNA, 23 bp, artificial
  synthetic
  modified_base (2)..(2): c is methyl-cytosine
  modified_base (14)..(14): c is methyl-cytosine
actgttatcagctcagtaggcac

SEQ ID NO: 95 - DNA, 22 bp, artificial
  synthetic
  modified_base (4)..(4): c is methyl-cytosine
  modified_base (7)..(7): c is methyl-cytosine
  modified_base (16)..(16): c is methyl-cytosine
  modified_base (19)..(19): c is methyl-cytosine
tatctgcactaaatgcacctta

SEQ ID NO: 96 - DNA, 22 bp, artificial
  synthetic
  modified_base (8)..(8): c is methyl-cytosine
  modified_base (17)..(17): c is methyl-cytosine
  modified_base (20)..(20): c is methyl-cytosine
cacaaaccattctgtgctgcta

SEQ ID NO: 97 - DNA, 23 bp, artificial
  synthetic
  modified_base (5)..(5): c is methyl-cytosine
  modified_base (8)..(8): c is methyl-cytosine
  modified_base (17)..(17): c is methyl-cytosine
  modified_base (20)..(20): c is methyl-cytosine
caatcagctaacaacactgccta

SEQ ID NO: 98 - DNA, 22 bp, artificial
  synthetic
  modified_base (19)..(19): c is methyl-cytosine
acaaagttctgtaatgcactga

SEQ ID NO: 99 - DNA, 22 bp, artificial
  synthetic
  modified_base (3)..(3): c is methyl-cytosine
  modified_base (15)..(15): c is methyl-cytosine
cacaggttaagggtctcaggga

SEQ ID NO: 100 - DNA, 18 bp, artificial
  synthetic
  modified_base (6)..(6): c is methyl-cytosine
  modified_base (12)..(12): c is methyl-cytosine
agacacatgcactgtaga

SEQ ID NO: 101 - DNA, 22 bp, artificial
  synthetic
cactggtacaaggattgggaga

SEQ ID NO: 102 - DNA, 20 bp, artificial
  synthetic
  modified_base (7)..(7): c is methyl-cytosine
  modified_base (19)..(19): c is methyl-cytosine
ggctgtcaattcataggtca

SEQ ID NO: 103 - DNA, 22 bp, artificial
  synthetic
  modified_base (19)..(19): c is methyl-cytosine
aacaacacaacttactacctca

SEQ ID NO: 104 - DNA, 21 bp, artificial
  synthetic
  modified_base (2)..(2): c is methyl-cytosine
  modified_base (11)..(11): c is methyl-cytosine
  modified_base (14)..(14): c is methyl-cytosine
  modified_base (17)..(17): c is methyl-cytosine
  modified_base (20)..(20): c is methyl-cytosine
actgtacaaacaactacctca

SEQ ID NO: 105 - DNA, 23 bp, artificial
  synthetic
  modified_base (10)..(10): c is methyl-cytosine
  modified_base (22)..(22): c is methyl-cytosine
gcttccagtcggggatgtttaca

SEQ ID NO: 106 - DNA, 22 bp, artificial
  synthetic
  modified_base (4)..(4): c is methyl-cytosine
aacctatcctggattacttgaa

SEQ ID NO: 107 - DNA, 21 bp, artificial
  synthetic
  modified_base (11)..(11): c is methyl-cytosine
caacaccagtctgataagcta

SEQ ID NO: 108 - DNA, 21 bp, artificial
  synthetic
  modified_base (4)..(4): c is methyl-cytosine
  modified_base (13)..(13): c is methyl-cytosine
acccttggaattcagttctca

SEQ ID NO: 109 - DNA, 22 bp, artificial
  synthetic
  modified_base (13)..(13): c is methyl-cytosine
  modified_base (19)..(19): c is methyl-cytosine
tgtgagttctaccattgccaaa

SEQ ID NO: 110 - DNA, 21 bp, artificial
  synthetic
tagttggcaagtctagaacca

SEQ ID NO: 111 - DNA, 21 bp, artificial
  synthetic
  modified_base (8)..(8): c is methyl-cytosine
aagtgtccgatacggttgtgg

SEQ ID NO: 112 - DNA, 23 bp, artificial
  synthetic
  modified_base (3)..(3): c is methyl-cytosine
  modified_base (12)..(12): c is methyl-cytosine
gcccatcaaagctggctgtgata

SEQ ID NO: 113 - DNA, 22 bp, artificial
  synthetic
  modified_base (13)..(13): c is methyl-cytosine
aaaaagaacagccactgtgata

SEQ ID NO: 114 - DNA, 23 bp, artificial
  synthetic
  modified_base (2)..(2): c is methyl-cytosine
  modified_base (5)..(5): c is methyl-cytosine
  modified_base (11)..(11): c is methyl-cytosine
acaacaaaatcactagtcttcca

SEQ ID NO: 115 - DNA, 21 bp, artificial
  synthetic
taggagagagaaaaagactga

SEQ ID NO: 116 - DNA, 23 bp, artificial
  synthetic
  modified_base (9)..(9): c is methyl-cytosine
  modified_base (18)..(18): c is methyl-cytosine
tgtcgtaccagatagtgcattta

SEQ ID NO: 117 - DNA, 22 bp, artificial
  synthetic
  modified_base (15)..(15): c is methyl-cytosine
aaacggacgaaagtcccaccga

SEQ ID NO: 118 - DNA, 22 bp, artificial
  synthetic
ttaatgagtgtggatctagtca

SEQ ID NO: 119 - DNA, 22 bp, artificial
  synthetic
  modified_base (17)..(17): c is methyl-cytosine
taggacaaactttacccagtgc

SEQ ID NO: 120 - DNA, 22 bp, artificial
  synthetic
aaaggccgggaagtgtgcaata

SEQ ID NO: 121 - DNA, 22 bp, artificial
  synthetic
  modified_base (10)..(10): c is methyl-cytosine
  modified_base (16)..(16): c is methyl-cytosine
tgagataaacaaagcccagtga

SEQ ID NO: 122 - DNA, 23 bp, artificial
  synthetic
aatcagctttcaaaatgatctca

SEQ ID NO: 123 - DNA, 19 bp, artificial
  synthetic
  modified_base (5)..(5): c is methyl-cytosine
  modified_base (8)..(8): c is methyl-cytosine
  modified_base (17)..(17): c is methyl-cytosine
catacttctttacattcca

SEQ ID NO: 124 - DNA, 18 bp, artificial
  synthetic
  modified_base (7)..(7): c is methyl-cytosine
tcaaagctggctgtgata

SEQ ID NO: 125 - DNA, 18 bp, artificial
  synthetic
  modified_base (6)..(6): c is methyl-cytosine
tcacacttgaggtctcag

SEQ ID NO: 126 - DNA, 21 bp, artificial
  synthetic
  modified_base (14)..(14): c is methyl-cytosine
  modified_base (16)..(16): c is methyl-cytosine
  modified_base (20)..(20): c is methyl-cytosine
gatattggcgcggctcaatca

SEQ ID NO: 127 - DNA, 21 bp, artificial
  synthetic
  modified_base (11)..(11): c is methyl-cytosine
  modified_base (14)..(14): c is methyl-cytosine
  modified_base (20)..(20): c is methyl-cytosine
gatattggcgcggctcaatca

SEQ ID NO: 128 - DNA, 21 bp, artificial
  synthetic
  modified_base (6)..(6): c is methyl-cytosine
  modified_base (8)..(8): c is methyl-cytosine
  modified_base (10)..(10): c is methyl-cytosine
  modified_base (18)..(18): c is methyl-cytosine
tagagctcccttcaatccaaa

SEQ ID NO: 129 - DNA, 21 bp, artificial
  synthetic
  modified_base (8)..(8): c is methyl-cytosine
  modified_base (17)..(17): c is methyl-cytosine
tagagctcccttcaatccaaa

SEQ ID NO: 130 - DNA, 21 bp, artificial
  synthetic
  modified_base (2)..(2): c is methyl-cytosine
ccccgatgtagtcactttcaa

SEQ ID NO: 131 - DNA, 21 bp, artificial
  synthetic
  modified_base (14)..(14): c is methyl-cytosine
  modified_base (17)..(17): c is methyl-cytosine
  modified_base (20)..(20): c is methyl-cytosine
tagatcatgctggcagcttca

SEQ ID NO: 132 - DNA, 20 bp, artificial
  synthetic
  modified_base (6)..(6): c is methyl-cytosine
  modified_base (9)..(9): c is methyl-cytosine
  modified_base (18)..(18): c is methyl-cytosine
gggagctcccttcagtccaa

SEQ ID NO: 133 - DNA, 21 bp, artificial
  synthetic
  modified_base (8)..(8): c is methyl-cytosine
  modified_base (17)..(17): c is methyl-cytosine
tagagctcccttcaatccaaa

SEQ ID NO: 134 - DNA, 20 bp, artificial
  synthetic
  modified_base (6)..(6): c is methyl-cytosine
  modified_base (9)..(9): c is methyl-cytosine
  modified_base (18)..(18): c is methyl-cytosine
gggagctcccttcagtccaa

SEQ ID NO: 135 - DNA, 19 bp, artificial
  synthetic
gttcaagaaagctgtggaa

SEQ ID NO: 136 - DNA, 20 bp, artificial
  synthetic
  modified_base (4)..(4): c is methyl-cytosine
  modified_base (10)..(10): c is methyl-cytosine
  modified_base (19)..(19): c is methyl-cytosine
gtgctcactctcttctgtca

SEQ ID NO: 137 - DNA, 21 bp, artificial
  synthetic
  modified_base (4)..(4): c is methyl-cytosine
  modified_base (7)..(7): c is methyl-cytosine
  modified_base (10)..(10): c is methyl-cytosine
  modified_base (13)..(13): c is methyl-cytosine
atgcagcatcatcaagattct

SEQ ID NO: 138 - DNA, 22 bp, artificial
  synthetic
  modified_base (4)..(4): c is methyl-cytosine
  modified_base (10)..(10): c is methyl-cytosine
  modified_base (16)..(16): c is methyl-cytosine
ccacacactctcttacattcca

SEQ ID NO: 139 - DNA, 22 bp, artificial
  synthetic
  modified_base (4)..(4): c is methyl-cytosine
  modified_base (10)..(10): c is methyl-cytosine
  modified_base (16)..(16): c is methyl-cytosine
ccacacactcccttacattcca

SEQ ID NO: 140 - DNA, 22 bp, artificial
  synthetic
  modified_base (4)..(4): c is methyl-cytosine
  modified_base (13)..(13): c is methyl-cytosine
tggcattcaaagcgtgccttaa

SEQ ID NO: 141 - DNA, 22 bp, artificial
  synthetic
  modified_base (4)..(4): c is methyl-cytosine
  modified_base (13)..(13): c is methyl-cytosine
tggcattcaacgcgtgccttaa

SEQ ID NO: 142 - DNA, 23 bp, artificial
  synthetic
  modified_base (6)..(6): c is methyl-cytosine
  modified_base (9)..(9): c is methyl-cytosine
  modified_base (12)..(12): c is methyl-cytosine
  modified_base (15)..(15): c is methyl-cytosine
  modified_base (21)..(21): c is methyl-cytosine
acaaacaccaccgtcacactcca

SEQ ID NO: 143 - DNA, 23 bp, artificial
  synthetic
  modified_base (6)..(6): c is methyl-cytosine
  modified_base (9)..(9): c is methyl-cytosine
  modified_base (12)..(12): c is methyl-cytosine
  modified_base (15)..(15): c is methyl-cytosine
  modified_base (21)..(21): c is methyl-cytosine
acaaacaccatcgtcacactcca

SEQ ID NO: 144 - DNA, 22 bp, artificial
  synthetic
  modified_base (3)..(3): c is methyl-cytosine
  modified_base (12)..(12): c is methyl-cytosine
  modified_base (15)..(15): c is methyl-cytosine
  modified_base (18)..(18): c is methyl-cytosine
  modified_base (21)..(21): c is methyl-cytosine
aactatacaacctactacctca

SEQ ID NO: 145 - DNA, 22 bp, artificial
  synthetic
  modified_base (3)..(3): c is methyl-cytosine
  modified_base (12)..(12): c is methyl-cytosine
  modified_base (15)..(15): c is methyl-cytosine
  modified_base (18)..(18): c is methyl-cytosine
  modified_base (21)..(21): c is methyl-cytosine
aactatacaatctactacctca

SEQ ID NO: 146 - DNA, 22 bp, artificial
  synthetic
  modified_base (5)..(5): c is methyl-cytosine
  modified_base (8)..(8): c is methyl-cytosine
tgagctacagtgcttcatctca

SEQ ID NO: 147 - DNA, 24 bp, artificial
  synthetic
aagggattcctgggaaaactggac

SEQ ID NO: 148 - DNA, 23 bp, artificial
  synthetic
  modified_base (5)..(5): c is methyl-cytosine
  modified_base (11)..(11): c is methyl-cytosine
  modified_base (14)..(14): c is methyl-cytosine
ttcgccctctcaacccagctttt

SEQ ID NO: 149 - DNA, 22 bp, artificial
  synthetic
  modified_base (4)..(4): c is methyl-cytosine
agcctatcctggattacttgaa

SEQ ID NO: 150 - DNA, 22 bp, artificial
  synthetic
cacaagatcggatctacgggtt

SEQ ID NO: 151 - DNA, 22 bp, artificial
  synthetic
  modified_base (8)..(8): c is methyl-cytosine
  modified_base (17)..(17): c is methyl-cytosine
  modified_base (20)..(20): c is methyl-cytosine
cacaaaccattatgtgctgcta

SEQ ID NO: 152 - DNA, 22 bp, artificial
  synthetic
  modified_base (3)..(3): c is methyl-cytosine
cgccaatatttacgtgctgcta

SEQ ID NO: 153 - DNA, 22 bp, artificial
  synthetic
  modified_base (20)..(20): c is methyl-cytosine
ctgttcctgctgaactgagcca

SEQ ID NO: 154 - DNA, 21 bp, artificial
  synthetic
  modified_base (2)..(2): c is methyl-cytosine
  modified_base (11)..(11): c is methyl-cytosine
  modified_base (14)..(14): c is methyl-cytosine
  modified_base (17)..(17): c is methyl-cytosine
  modified_base (20)..(20): c is methyl-cytosine
actgtacaaactactacctca

SEQ ID NO: 155 - DNA, 22 bp, artificial
  synthetic
  modified_base (3)..(3): c is methyl-cytosine
  modified_base (15)..(15): c is methyl-cytosine
  modified_base (18)..(18): c is methyl-cytosine
  modified_base (21)..(21): c is methyl-cytosine
aactatacaacatactacctca

SEQ ID NO: 156 - DNA, 22 bp, artificial
  synthetic
  modified_base (3)..(3): c is methyl-cytosine
  modified_base (15)..(15): c is methyl-cytosine
  modified_base (18)..(18): c is methyl-cytosine
  modified_base (21)..(21): c is methyl-cytosine
aactatacaatatactacctca

SEQ ID NO: 157 - DNA, 22 bp, artificial
  synthetic
  modified_base (5)..(5): c is methyl-cytosine
  modified_base (8)..(8): c is methyl-cytosine
  modified_base (11)..(11): c is methyl-cytosine
tgagctacagcgcttcatctca

SEQ ID NO: 158 - DNA, 24 bp, artificial
  synthetic
aagggattcctcggaaaactggac

SEQ ID NO: 159 - DNA, 23 bp, artificial
  synthetic
  modified_base (5)..(5): c is methyl-cytosine
  modified_base (14)..(14): c is methyl-cytosine
ttcgccctctaaacccagctttt

SEQ ID NO: 160 - DNA, 22 bp, artificial
  synthetic
  modified_base (4)..(4): c is methyl-cytosine
agcctatcctcgattacttgaa

SEQ ID NO: 161 - DNA, 22 bp, artificial
  synthetic
cacaagatcgcatctacgggtt

SEQ ID NO: 162 - DNA, 22 bp, artificial
  synthetic
  modified_base (8)..(8): c is methyl-cytosine
  modified_base (11)..(11): c is methyl-cytosine
  modified_base (17)..(17): c is methyl-cytosine
  modified_base (20)..(20): c is methyl-cytosine
cacaaaccatcatgtgctgcta

SEQ ID NO: 163 - DNA, 22 bp, artificial
  synthetic
  modified_base (3)..(3): c is methyl-cytosine
cgccaatattttcgtgctgcta

SEQ ID NO: 164 - DNA, 22 bp, artificial
  synthetic
  modified_base (11)..(11): c is methyl-cytosine
  modified_base (20)..(20): c is methyl-cytosine
ctgttcctgccgaactgagcca

SEQ ID NO: 165 - DNA, 21 bp, artificial
  synthetic
  modified_base (2)..(2): c is methyl-cytosine
  modified_base (14)..(14): c is methyl-cytosine
  modified_base (17)..(17): c is methyl-cytosine
  modified_base (20)..(20): c is methyl-cytosine
actgtacaaaatactacctca

SEQ ID NO: 166 - DNA, 22 bp, artificial
  synthetic
tgaggtagtaggttgtatagtt

SEQ ID NO: 167 - DNA, 21 bp, artificial
  synthetic
tggaatgtaaagaagtatgta

SEQ ID NO: 168 - DNA, 22 bp, artificial
  synthetic
  modified_base (4)..(4): c is methyl-cytosine
  modified_base (7)..(7): c is methyl-cytosine
tagcagcacgtaaatattggcg

SEQ ID NO: 169 - DNA, 22 bp, artificial
  synthetic
  modified_base (4)..(4): c is methyl-cytosine
  modified_base (7)..(7): c is methyl-cytosine
  modified_base (19)..(19): c is methyl-cytosine
aagctgccagttgaagaactgt

SEQ ID NO: 170 - DNA, 22 bp, artificial
  synthetic
ttcaagtaattcaggataggtt

SEQ ID NO: 171 - DNA, 23 bp, artificial
  synthetic
  modified_base (7)..(7): c is methyl-cytosine
  modified_base (10)..(10): c is methyl-cytosine
  modified_base (16)..(16): c is methyl-cytosine
tgtaaacatcctacactctcagc

SEQ ID NO: 172 - DNA, 22 bp, artificial
  synthetic
tggagtgtgacaatggtgtttg

SEQ ID NO: 173 - DNA, 21 bp, artificial
  synthetic
cattattacttttggtacgcg

SEQ ID NO: 174 - DNA, 21 bp, artificial
  synthetic
  modified_base (7)..(7): c is methyl-cytosine
tcgtaccgtgagtaataatgc

SEQ ID NO: 175 - DNA, 22 bp, artificial
  synthetic
  modified_base (8)..(8): c is methyl-cytosine
ttggtccccttcaaccagctgt

SEQ ID NO: 176 - DNA, 22 bp, artificial
  synthetic
  modified_base (11)..(11): c is methyl-cytosine
tgagatgaagcactgtagctca

SEQ ID NO: 177 - DNA, 22 bp, artificial
  synthetic
tacagtatagatgatgtactag

SEQ ID NO: 178 - DNA, 22 bp, artificial
  synthetic
  modified_base (3)..(3): c is methyl-cytosine
  modified_base (12)..(12): c is methyl-cytosine
  modified_base (15)..(15): c is methyl-cytosine
  modified_base (18)..(18): c is methyl-cytosine
  modified_base (21)..(21): c is methyl-cytosine
aactatacaacctactacctca

SEQ ID NO: 179 - DNA, 21 bp, artificial
  synthetic
  modified_base (3)..(3): c is methyl-cytosine
  modified_base (15)..(15): c is methyl-cytosine
tacatacttctttacattcca

SEQ ID NO: 180 - DNA, 22 bp, artificial
  synthetic
  modified_base (3)..(3): c is methyl-cytosine
cgccaatatttacgtgctgcta

SEQ ID NO: 181 - DNA, 22 bp, artificial
  synthetic
  modified_base (7)..(7): c is methyl-cytosine
  modified_base (10)..(10): c is methyl-cytosine
  modified_base (13)..(13): c is methyl-cytosine
acagttcttcaactggcagctt

SEQ ID NO: 182 - DNA, 22 bp, artificial
  synthetic
  modified_base (4)..(4): c is methyl Cytosine
aacctatcctgaattacttgaa

SEQ ID NO: 183 - DNA, 23 bp, artificial
  synthetic
  modified_base (2)..(2): c is methyl-cytosine
gctgagagtgtaggatgtttaca

SEQ ID NO: 184 - DNA, 22 bp, artificial
  synthetic
  modified_base (5)..(5): c is methyl-cytosine
  modified_base (8)..(8): c is methyl-cytosine
  modified_base (14)..(14): c is methyl-cytosine
  modified_base (20)..(20): c is methyl-cytosine
caaacaccattgtcacactcca

SEQ ID NO: 185 - DNA, 21 bp, artificial
  synthetic
  modified_base (3)..(3): c is methyl-cytosine
cgcgtaccaaaagtaataatg

SEQ ID NO: 186 - DNA, 21 bp, artificial
  synthetic
  modified_base (12)..(12): c is methyl-cytosine
gcattattactcacggtacga

SEQ ID NO: 187 - DNA, 22 bp, artificial
  synthetic
acagctggttgaaggggaccaa

SEQ ID NO: 188 - DNA, 22 bp, artificial
  synthetic
  modified_base (5)..(5): c is methyl-cytosine
  modified_base (8)..(8): c is methyl-cytosine
tgagctacagtgcttcatctca

SEQ ID NO: 189 - DNA, 22 bp, artificial
  synthetic
  modified_base (7)..(7): c is methyl-cytosine
  modified_base (10)..(10): c is methyl-cytosine
  modified_base (13)..(13): c is methyl-cytosine
ctagtacatcatctatactgta

SEQ ID NO: 190 - DNA, 22 bp, artificial
  synthetic
tgaggtagtaagttgtatagtt

SEQ ID NO: 191 - DNA, 21 bp, artificial
  synthetic
tggaatgtaaggaagtatgta

SEQ ID NO: 192 - DNA, 22 bp, artificial
  synthetic
tagcagcacggaaatattggcg

SEQ ID NO: 193 - DNA, 22 bp, artificial
  synthetic
aagctgccaggtgaagaactgt

SEQ ID NO: 194 - DNA, 22 bp, artificial
  synthetic
ttcaagtaatgcaggataggtt

SEQ ID NO: 195 - DNA, 23 bp, artificial
  synthetic
  modified_base (7)..(7): c is methyl-cytosine
  modified_base (10)..(10): c is methyl-cytosine
  modified_base (16)..(16): c is methyl-cytosine
tgtaaacatcatacactctcagc

SEQ ID NO: 196 - DNA, 22 bp, artificial
  synthetic
tggagtgtgaaaatggtgtttg

SEQ ID NO: 197 - DNA, 21 bp, artificial
  synthetic
cattattactgttggtacgcg

SEQ ID NO: 198 - DNA, 21 bp, artificial
  synthetic
  modified_base (2)..(2): c is methyl-cytosine
tcgtaccgtgggtaataatgc

SEQ ID NO: 199 - DNA, 22 bp, artificial
  synthetic
  modified_base (7)..(7): c is methyl-cytosine
  modified_base (16)..(16): c is methyl-cytosine
  modified_base (19)..(19): c is methyl-cytosine
ttggtcccctgcaaccagctgt

SEQ ID NO: 200 - DNA, 22 bp, artificial
  synthetic
tgagatgaagaactgtagctca

SEQ ID NO: 201 - DNA, 22 bp, artificial
  synthetic
tacagtataggtgatgtactag

SEQ ID NO: 202 - DNA, 22 bp, artificial
  synthetic
  modified_base (8)..(8): c is methyl-cytosine
  modified_base (11)..(11): c is methyl-cytosine
aactatacaacttactacctca

SEQ ID NO: 203 - DNA, 21 bp, artificial
  synthetic
  modified_base (7)..(7): c is methyl-cytosine
  modified_base (10)..(10): c is methyl-cytosine
  modified_base (19)..(19): c is methyl-cytosine
tacatacttccttacattcca

SEQ ID NO: 204 - DNA, 22 bp, artificial
  synthetic
  modified_base (3)..(3): c is methyl-cytosine
  modified_base (12)..(12): c is methyl-cytosine
cgccaatatttccgtgctgcta

SEQ ID NO: 205 - DNA, 22 bp, artificial
  synthetic
  modified_base (2)..(2): c is methyl-cytosine
  modified_base (17)..(17): c is methyl-cytosine
  modified_base (20)..(20): c is methyl-cytosine
acagttcttcacctggcagctt

SEQ ID NO: 206 - DNA, 22 bp, artificial
  synthetic
  modified_base (3)..(3): c is methyl-cytosine
  modified_base (9)..(9): c is methyl-cytosine
  modified_base (12)..(12): c is methyl-cytosine
aacctatcctgcattacttgaa

SEQ ID NO: 207 - DNA, 23 bp, artificial
  synthetic
  modified_base (2)..(2): c is methyl-cytosine
gctgagagtgtatgatgtttaca

SEQ ID NO: 208 - DNA, 22 bp, artificial
  synthetic
  modified_base (5)..(5): c is methyl-cytosine
  modified_base (8)..(8): c is methyl-cytosine
  modified_base (14)..(14): c is methyl-cytosine
  modified_base (20)..(20): c is methyl-cytosine
caaacaccattttcacactcca

SEQ ID NO: 209 - DNA, 21 bp, artificial
  synthetic
  modified_base (3)..(3): c is methyl-cytosine
cgcgtaccaacagtaataatg

SEQ ID NO: 210 - DNA, 21 bp, artificial
  synthetic
  modified_base (2)..(2): c is methyl-cytosine
  modified_base (11)..(11): c is methyl-cytosine
  modified_base (14)..(14): c is methyl-cytosine
gcattattacccacggtacga

SEQ ID NO: 211 - DNA, 22 bp, Artificial Sequence
  synthetic
  modified_base (19)..(19): c is methyl-cytosine
acagctggttgcaggggaccaa

SEQ ID NO: 212 - DNA, 22 bp, Artificial Sequence
  synthetic
tgagctacagttcttcatctca

SEQ ID NO: 213 - DNA, 22 bp, Artificial Sequence
  synthetic
ctagtacatcacctatactgta

SEQ ID NO: 214 - DNA, 21 bp, artificial sequence
  synthetic
gagcucggccaacgucuactt

SEQ ID NO: 215 - DNA, 20 bp, artificial sequence
  synthetic
tctctaaggctccagcctac

SEQ ID NO: 216 - DNA, 20 bp, artificial sequence
  synthetic
gcttaaccacggagatctga

SEQ ID NO: 217 - DNA, 20 bp, artificial sequence
  synthetic
caccatgccgttcggcaaca

SEQ ID NO: 218 - DNA, 18 bp, artificial sequence
  synthetic
ggttgtccaccccagtct

SEQ ID NO: 219 - DNA, 22 bp, artificial sequence
  synthetic
atgtgcagctgataaagactgg

SEQ ID NO: 220 - DNA, 22 bp, artificial sequence
  synthetic
aggccttgaccttttcagtaag

SEQ ID NO: 221 - RNA, 23 nt, artificial sequence
  synthetic
aacauucaacgcugucggugagu

SEQ ID NO: 222 - RNA, 23 nt, artificial sequence
  synthetic
aacauucaacgcugucggugagu

SEQ ID NO: 223 - RNA, 23 nt, artificial sequence
  synthetic
aacauucaacgcugucggugagu

SEQ ID NO: 224 - DNA, 22 bp, Artificial Sequence
  synthetic
tcacaagttagggtctcaggga

SEQ ID NO: 225 - DNA, 22 bp, Artificial Sequence
  synthetic
catgtcatgtgtcacatctctt

SEQ ID NO: 226 - DNA, 23 bp, Artificial Sequence
  synthetic
actcaccgacagcgttgaatgtt

SEQ ID NO: 227 - DNA, 23 bp, Artificial Sequence
  synthetic
actcacggtccgagttgaatgtt

SEQ ID NO: 228 - DNA, 22 bp, Artificial Sequence
  synthetic
uugggauucaaaaacaaaaatt

SEQ ID NO: 229 - DNA, 22 bp, Artificial Sequence
  synthetic
ttaaccccaaguuuuuguuuuu

SEQ ID NO: 230 - DNA, 22 bp, Artificial Sequence
  synthetic
uuggcauucgaaaucagaaatt

SEQ ID NO: 231 - DNA, 22 bp, Artificial Sequence
  synthetic
ttaaccgcaagcuuuagucuuu

SEQ ID NO: 232 - RNA, 23 nt, Artificial Sequence
  synthetic
aacauucaacgcugucggugagu

SEQ ID NO: 233 - DNA, 43 bp, Artificial Sequence
  synthetic
gttccaaggttaaactcgctcactgctaaaatatttgaatgta

SEQ ID NO: 234 - DNA, 43 bp, Artificial Sequence
  synthetic
gttccaaggttaaagtcgctcactgctaaaatatttatcagta

SEQ ID NO: 235 - DNA, 23 bp, Artificial Sequence
  synthetic
ccuuguaaguugcgacagccacu

SEQ ID NO: 236 - DNA, 23 bp, Artificial Sequence
  synthetic
ccauguaaguugcgacagccacu

SEQ ID NO: 237 - DNA, 20 bp, Artificial Sequence
  synthetic
ccaugucaguugccagccac

(19) United States
(12) Patent Application Publication
(10) Pub. No.: US 2007/0065840 A1
(43) Pub. Date: Mar. 22, 2007

(54) Novel oligonucleotide compositions and probe sequences useful for detection and analysis of microRNAS and their target mRNAS

(76) Inventors: Irena Naguibneva, (US); Annick Harel-Bellan, (US); Anders Lund, (US); Soren Morgonthaler Echwald, (US)

Correspondence Address: CLARK & ELBING LLP, 101 FEDERAL STREET, BOSTON, MA, 02110, US

(21) Appl. No.: 11/388,079

(22) Filed: Mar. 23, 2006

Related U.S. Application Data
(60) Provisional application No. 60/664,566, filed on Mar. 23, 2005.

(51) Int. Cl. C12Q 1/68; C07H 21/04

(52) U.S. Cl. 435/6; 536/24.1

(57) ABSTRACT

The invention relates to ribonucleic acids and oligonucleotide probes useful for detection and analysis of microRNAs and their target mRNAs, as well as small interfering RNAs (siRNAs). The invention furthermore relates to oligonucleotide probes for detection and analysis of other non-coding RNAs, mRNAs, mRNA splice variants, allelic variants of single transcripts, mutations, deletions, or duplications of particular exons in transcripts, e.g. alterations associated with human disease, such as cancer.

### CROSS-REFERENCE TO RELATED APPLICATIONS

This application claims benefit of U.S. Provisional Application No. 60/664,566, filed Mar. 23, 2005, which is hereby incorporated by reference.

### BACKGROUND OF THE INVENTION

The present invention relates to ribonucleic acids and oligonucleotide probes useful for detection and analysis of microRNAs and their target mRNAs, as well as small interfering RNAs (siRNAs). The invention furthermore relates to oligonucleotide probes for detection and analysis of other non-coding RNAs, as well as mRNAs, mRNA splice variants, allelic variants of single transcripts, mutations, deletions, or duplications of particular exons in transcripts, e.g. alterations associated with human disease, such as cancer.

The present invention relates to the detection and analysis of target nucleo-tide sequences in a wide variety of nucleic acid samples and more specifically to the methods employing the design and use of oligonucleotide probes that are useful for detecting and analysing target nucleotide sequences, especially RNA target sequences, such as microRNAs and their target mRNAs and siRNA sequences of interest and for detecting differences between nucleic acid samples (e.g., such as samples from a cancer patient and a healthy patient).

MicroRNAs

The expanding inventory of international sequence databases and the con-comitant sequencing of nearly 200 genomes representing all three domains of life—bacteria, archea and eukaryota—have been the primary drivers in the process of de-constructing living organisms into comprehensive molecular catalogs of genes, transcripts and proteins. The importance of the genetic variation within a single species has become apparent, extending beyond the completion of genetic blueprints of several important genomes, culminating in the publication of the working draft of the human genome sequence in 2001 (Lander, Linton, Birren et aL, 2001 Nature 409: 860-921; Venter, Adams, Myers et al., 2001 Science 291: 1304-1351; Sachidanandam, Weissman, Schmidt et al., 2001 Nature 409: 928-933). On the other hand, the increasing number of detailed, large-scale molecular analyses of transcription originating from the human and mouse genomes along with the recent identification of several types of non-protein-coding RNAs, such as small nuclear RNAs, siRNAs, microRNAs and antisense RNAs, indicate that the transcriptomes of higher eukaryotes are much more complex than originally anticipated (Wong et al. 2001, Genome Research 11: 1975-1977; Kampa etal. 2004, Genome Research 14: 331-342).

As a result of the Central Dogma: ‘DNA makes RNA, and RNA makes protein’, RNAs have been considered as simple molecules that just translate the genetic information into protein. Recently, it has been estimated that although most of the genome is transcribed, almost 97% of the genome does not encode proteins in higher eukaryotes, but putative, non-coding RNAs (Wong et al. 2001, Genome Research 11: 1975-1977). The non-coding RNAs (ncRNAs) appear to be particularly well suited for regulatory roles that require highly specific nucleic acid recognition. Therefore, the view of RNA is rapidly changing from the merely informational molecule to comprise a wide variety of structural, informational and catalytic molecules in the cell.

Recently, a large number of small non-coding RNA genes have been identified and designated as microRNAs (miRNAs) (for review, see Ke et al. 2003, Curr.Opin. Chem. Biol. 7:516-523). The first miRNAs to be discovered were the lin-4 and let-7 that are heterochronic switching genes essential for the normal temporal control of diverse developmental events (Lee et al. 1993, Cell 75:843-854; Reinhart et al. 2000, Nature 403: 901-906) in the roundworm C. elegans. miRNAs have been evolutionarily conserved over a wide range of species and exhibit diversity in expression profiles, suggesting that they occupy a wide variety of regulatory functions and exert significant effects on cell growth and development (Ke et al. 2003, Curr.Opin. Chem. Biol. 7:516-523). Recent work has shown that miRNAs can regulate gene expression at many levels, representing a novel gene regulatory mechanism and supporting the idea that RNA is capable of performing similar regulatory roles as proteins. Understanding this RNA-based regulation will help us to understand the complexity of the genome in higher eukaryotes as well as understand the complex gene regulatory networks.

miRNAs are 19-25 nucleotide (nt) RNAs that are processed from longer endogenous hairpin transcripts (Ambros et aL 2003, RNA 9: 277-279). To date more than 1345 microRNAs have been identified in humans, worms, fruit flies and plants according to the miRNA registry database release 5.0 in September 2004, hosted by Sanger Institute, UK, and many miRNAs that correspond to putative genes have also been identified. Some miRNAs have multiple loci in the genome (Reinhart et al. 2002, Genes Dev. 16: 1616-1626) and occasionally, several miRNA genes are arranged in tandem clusters (Lagos-Quintana et al. 2001, Science 294: 853-858). The fact that many of the miRNAs reported to date have been isolated just once suggests that many new miRNAs will be discovered in the future. A recent in-depth transcriptional analysis of the human chromosomes 21 and 22 found that 49% of the observed transcription was outside of any known annotation, and furthermore, that these novel transcripts were both coding and non-coding RNAs (Kampa et aL. 2004, Genome Research 14: 331-342). The identified miRNAs to date represent most likely the tip of the iceberg, and the number of miRNAs might turn out to be very large.

The combined characteristics of microRNAs characterized to date (Ke et al. 2003, Curr.Opin. Chem. Biol. 7:516-523; Lee et al. 1993, Cell 75:843-854; Reinhart et aL. 2000, Nature 403: 901-906) can be summarized as:
1. miRNAs are single-stranded RNAs of about 19-25 nt that regulate the expression of complementary messenger RNAs
2. They are cleaved from a longer endogenous double-stranded hairpin precursor by the enzyme Dicer.
3. miRNAs match precisely the genomic regions that can potentially encode precursor RNAs in the form of double-stranded hairpins.
4. miRNAs and their predicted precursor secondary structures are phylogenetically conserved.

Several lines of evidence suggest that the enzymes Dicer and Argonaute are crucial participants in miRNA biosynthesis, maturation and function (Grishok et al. 2001, Cell 106: 23-24). Mutations in genes required for miRNA biosynthesis lead to genetic developmental defects, which are, at least in part, derived from the role of generating miRNAs. The current view is that miRNAs are cleaved by Dicer from the hairpin precursor in the form of duplex, initially with 2 or 3 nt overhangs in the 3′ ends, and are termed pre-miRNAs. Cofactors join the pre-miRNP and unwind the pre-miRNAs into single-stranded miRNAs, and pre-miRNP is then transformed to miRNP. miRNAs can recognize regulatory targets while part of the miRNP complex. There are several similarities between miRNP and the RNA-induced silencing complex, RISC, including similar sizes and both containing RNA helicase and the PPD proteins. It has therefore been proposed that miRNP and RISC are the same RNP with multiple functions (Ke et al. 2003, Curr.Opin. Chem. Biol. 7:516-523). Different effectors direct miRNAs into diverse pathways. The structure of pre-miRNAs is consistent with the observation that 22 nt RNA duplexes with 2 or 3 nt overhangs at the 3′ ends are beneficial for reconstitution of the protein complex and might be required for high affinity binding of the short RNA duplex to the protein components (for review, see Ke et al. 2003, Curr.Opin. Chem. Biol. 7:516-523).

Growing evidence suggests that miRNAs play crucial roles in eukaryotic gene regulation. The first miRNAs genes to be discovered, lin-4 and let-7, base-pair incompletely to repeated elements in the 3′ untranslated regions (UTRs) of other heterochronic genes, and regulate the translation directly and negatively by antisense RNA-RNA interaction (Lee et al. 1993, Cell 75:843-854; Reinhart et al. 2000, Nature 403: 901-906). Other miRNAs are thought to interact with target mRNAs by limited complementary and suppressed translation as well (Lagos-Quintana et al. 2001, Science 294: 853-858; Lee and Ambros 2001, Science 294: 858-862). Many studies have shown, however, that given a perfect complementarity between miRNAs and their target RNA, could lead to target RNA degradation rather than inhibit translation (Hutvagner and Zamore 2002, Science 297: 2056-2060), suggesting that the degree of complementarity determines their functions. By identifying sequences with near complementarity, several targets have been predicted, most of which appear to be potential transcriptional factors that are crucial in cell growth and development. The high percentage of predicted miRNA targets acting as developmental regulators and the conservation of target sites suggest that miRNAs are involved in a wide range of organism development and behaviour and cell fate decisions (for review, see Ke et al. 2003, Curr.Opin. Chem. Biol. 7:516-523). For example, John et al. 2004 (PLoS Biology 2: e363) used known mammalian miRNAs to scan the 3′ untranslated regions (UTRs) from human, mouse and rat genomes for potential miRNA target sites using a scanning algorithm based on sequence complementarity between the mature miRNA and the target site, binding energy of the miRNA:mRNA duplex and evolutionary conservation. They identified a total of 2307 target mRNAs conserved across the mammals with more than one target site at 90% conservation of target site sequence and 660 target genes at 100% conservation level. Scanning of the two fish genomes; Danio rerio (zebrafish) and Fugu rubripes (Fugu) identified 1000 target genes with two or more conserved miRNA sites between the two fish species (John et al. 2004 PLoS Biology 2: e363). Among the predicted targets, particularly interesting groups included mRNA encoding transcription factors, components of the miRNA machinery, other proteins involved in the translational regulation as well as components of the ubiquitin machinery. Wang et al. 2004 (Genome Biology 5:R65) have developed and applied a computational algorithm to predict 95 Arabidopsis thaliana miRNAs, which included 12 known ones and 83 new miRNAs. The 83 new miRNAs were found to be conserved with more than 90% sequence identity between the Arabidopsis and rice genomes. Using the Smith-Waterman nucleotide-alignment algorithm to predict mRNA targets for the 83 new miRNAs and by focusing on target sites that were conserved in both Arabidopsis and rice, Wang et al. 2004 (Genome Biology 5:R65) predicted 371 mRNA targets with an average of 4.8 targets per miRNA. A large proportion of these mRNA targets encoded proteins with transcription regulatory activity.

MicroRNAs and Human Disease

Analysis of the genomic location of miRNAs indicates that they play important roles in human development and disease. Several human diseases have already been pinpointed in which miRNAs or their processing machinery might be implicated. One of them is spinal muscular atrophy (SMA), a paediatric neurodegenerative disease caused by reduced protein levels or loss-of-function mutations of the survival of motor neurons (SMN) gene (Paushkin et al. 2002, Curr.Opin.Cell Biol. 14: 305-312). Two proteins (Gemin3 and Gemin4) that are part of the SMN complex are also components of miRNPs, whereas it remains to be seen whether miRNA biogenesis or function is dysregulated in SMA and what effect this has on pathogenesis. Another neurological disease linked to mi/siRNAs is fragile X mental retardation (FXMR) caused by absence or mutations of the fragile X mental retardation protein (FMRP)(Nelson et al. 2003, TIBS 28: 534-540), and there are additional clues that miRNAs might play a role in other neurological diseases. Yet another interesting finding is that the miR-224 gene locus lies within the minimal candidate region of two different neurological diseases: early-onset Parkinsonism and X-linked mental retardation (Dostie et al. 2003, RNA: 9: 180-186). Links between cancer and miRNAs have also been recently described. The most frequent single genetic abnormality in chronic lymphocytic leukaemia (CLL) is a deletion localized to chromosome 13q14 (50% of the cases). A recent study determined that two different miRNA (miR15 and miR16) genes are clustered and located within the intron of LEU2, which lies within the deleted minimal region of the B-cell chronic lymphocytic leukaemia (B-CLL) tumour suppressor locus, and both genes are deleted or down-regulated in the majority of CLL cases (Calin et al. 2002, Proc. Natl. Acad. Sci.U.S.A. 99: 15524-15529). It has been anticipated that connections between miRNAs and human diseases will only strengthen in parallel with the knowledge of miRNAs and the gene networks that they control. Moreover, the understanding of the regulation of RNA-mediated gene expression is leading to the development of novel therapeutic approaches that will be likely to revolutionize the practice of medicine (Nelson et al. 2003, TIBS 28: 534-540).

Small Interfering RNAs and RNAi

Some of the recent attention paid to small RNAs in the size range of 21 to 25 nt is due to the phenomenon RNA interference (RNAi), in which double-stranded RNA leads to the degradation of any RNA that is homologous (Fire et al. 1998, Nature 391: 806-811). RNAi relies on a complex and ancient cellular mechanism that has probably evolved for protection against viral attack and mobile genetic elements. A crucial step in the RNAi mechanism is the generation of short interfering RNAs (siRNAs), double-stranded RNAs that are about 22 nt long each. The siRNAs lead to the degradation of homologous target RNA and the production of more siRNAs against the same target RNA (Lipardi et al. 2001, Cell 107: 297-307). The present view for the mRNA degradation pathway of RNAi is that antiparallel Dicer dimers cleave long double-stranded dsRNAs to form siRNAs in an ATP-dependent manner. The siRNAs are then incorporated in the RNA-induced silencing complex (RISC) and ATP-dependent unwinding of the siRNAs activates RISC (Zhang et al. 2002, EMBO J. 21: 5875-5885; Nykänen et al. 2001, Cell 107: 309-321). The active RISC complex is thus guided to degrade the specific target mRNAs.

Detection and Analysis of MicroRNAs and SiRNAs

The current view that miRNAs may represent a newly discovered, hidden layer of gene regulation has resulted in high interest among researchers around the world in the discovery of miRNAs, their targets and mechanism of action. Detection and analysis of these small RNAs is, however not trivial. Thus, the discovery of more than 1400 miRNAs to date has required taking advantage of their special features. First, the research groups have used the small size of the miRNAs as a primary criterion for isolation and detection. Consequently, standard cDNA libraries would lack miRNAs, primarily because RNAs that small are normally excluded by sixe selection in the cDNA library construction procedure. Total RNA from fly embryos, worms or HeLa cells have been size fractionated so that only molecules 25 nucleotides or smaller would be captured (Moss 2002, Curr.Biology 12: R138-R140). Synthetic oligomers have then been ligated directly to the RNA pools using T4 RNA ligase. Then the sequences have been reverse-transcribed, amplified by PCR, cloned and sequenced (Moss 2002, Curr.Biology 12: R138-R140). The genome databases have subsequently been queried with the sequences, confirming the origin of the miRNAs from these organisms as well as placing the miRNA genes physically in the context of other genes in the genome. The vast majority of the cloned sequences have been located in intronic regions or between genes, occasionally in clusters, suggesting that the tandemly arranged miRNAs are processed from a single transcript to allow coordinate regulation. Furthermore, the genomic sequences have revealed the fold-back structures of the miRNA precursors (Moss 2002, Curr.Biology 12: R138-R140).

The size and often low level of expression of different miRNAs require the use of sensitive and quantitative analysis tools. Due to their small size of 19-25 nt, the use of quantitative real-time PCR for monitoring expression of mature miRNAs is excluded. Therefore, most miRNA researchers currently use Northern blot analysis combined with polyacrylamide gels to examine expression of both the mature and pre-miRNAs (Reinhart et al. 2000, Nature 403: 901-906; Lagos-Quintana et al. 2001, Science 294: 853-858; Lee and Ambros 2001, Science 294: 862-864). Primer extension has also been used to detect the mature miRNA (Zeng and Cullen 2003, RNA 9: 112-123). The disadvantage of all the gel-based assays (Northern blotting, primer extension, RNase protection assays etc.) as tools for monitoring miRNA expression includes low throughput and poor sensitivity. Consequently, a large amount of total RNA per sample is required for Northern analysis of miRNAs, which is not feasible when the cell or tissue source is limited.

DNA microarrays would appear to be a good alternative to Northern blot analysis to quantify miRNAs in a genome-wide scale, since microarrays have excellent throughput. Krichevsky et al. 2003 used cDNA microarrays to monitor the expression of miRNAs during neuronal development with 5 to 10 μg aliquot of input total RNA as target, but the mature miRNAs had to be separated from the miRNA precursors using micro concentrators prior to microarray hybridizations (Krichevsky et al. 2003, RNA 9: 1274-1281). Liu et al. 2004 (Liu et al. 2004, Proc.Natl. Acad. Sci, U.S.A 101:9740-9744) have developed a microarray for expression profiling of 245 human and mouse miRNAs using 40-mer DNA oligonucleotide capture probes. Thomson et al. 2004 (Thomson et al. 2004, Nature Methods 1: 1-6) describe the development of a custom oligonucleotide microarray platform for expression profiling of 124 mammalian miRNAs conserved in human and mouse using oligonucleotide capture probes complementary to the mature microRNAs. The microarray was used in expression profiling of the 124 miRNAs in question in different adult mouse tissues and embryonic stages. A similar approach was used by Miska et al. 2004 (Genome Biology 2004; 5:R68) for the development of an oligoarray for expression profiling of 138 mammalian miRNAs, including 68 miRNAs from rat and monkey brains. Yet another approach was taken by Barad et al. 2004 (Genome Research 2004; 14: 2486-2494), who developed a 60-mer oligonucleotide microarray platform for known human mature miRNAs and their precursors. The drawback of all DNA-based oligonucleotide arrays regardless of the capture probe length is the requirement of high concentrations of labelled input target RNA for efficient hybridization and signal generation, low sensitivity for rare and low-abundant miRNAs, and the necessity for post-array validation using more sensitive assays such as real-time quantitative PCR, which is not currently feasible. In addition, at least in some array platforms discrimination of highly homologous miRNA differing by just one or two nucleotides could not be achieved, thus presenting problems in data interpretation, although the 60-mer microarray by Barad et al. 2004 (Genome Research 2004; 14: 2486-2494) appears to have adequate specificity.

A PCR approach has also been used to determine the expression levels of mature miRNAs (Grad et al. 2003, Mol. Cell 11: 1253-1263). This method is useful to clone miRNAs, but highly impractical for routine miRNA expression profiling, since it involves gel isolation of small RNAs and ligation to linker oligonucleotides. Allawi et al. (2004, RNA 10: 1153-1161) have developed a method for quantitation of mature miRNAs using a modified Invader assay. Although apparently sensitive and specific for the mature miRNA, the drawback of the Invader quantitation assay is the number of oligonucleotide probes and individual reaction steps needed for the complete assay, which increases the risk of cross-contamination between different assays and samples, especially when high-throughput analyses are desired. Schmittgen et al. (2004, Nucleic Acids Res. 32: e43) describe an alternative method to Northern blot analysis, in which they use real-time PCR assays to quantify the expression of miRNA precursors. The disadvantage of this method is that it only allows quantification of the precursor miRNAs, which does not necessarily reflect the expression levels of mature miRNAs. In order to fully characterize the expression of large numbers of miRNAs, it is necessary to quantify the mature miRNAs, such as those expressed in human disease, where alterations in miRNA biogenesis produce levels of mature miRNAs that are very different from those of the precursor miRNA. For example, the precursors of 26 miRNAs were equally expressed in non-cancerous and cancerous colorectal tissues from patients, whereas the expression of mature human miR143 and miR145 was greatly reduced in cancer tissues compared with non-cancer tissues, suggesting altered processing for specific miRNAs in human disease (Michael et al. 2003, Mol. Cancer Res. 1: 882-891). On the other hand, recent findings in maize with miR166 and miR165 in Arabidopsis thaliana, indicate that microRNAs act as signals to specify leaf polarity in plants and may even form movable signals that emanate from a signalling centre below the incipient leaf (Juarez et al. 2004, Nature 428: 84-88; Kidner and Martienssen 2004, Nature 428: 81-84).

Most of the miRNA expression studies in animals and plants have utilized Northern blot analysis, tissue-specific small RNA cloning and expression profiling by microarrays or real-time PCR of the miRNA hairpin precursors, as described above. However, these techniques lack the resolution for addressing the spatial and temporal expression patterns of mature miRNAs. Due to the small size of mature miRNAs, detection of them by standard RNA in situ hybridization has proven difficult to adapt in both plants and vertebrates, even though in situ hybridization has recently been reported in A. thaliana and maize using RNA probes corresponding to the stem-loop precursor miRNAs (Chen et al. 2004, Science 203: 2022-2025; Juarez et al. 2004, Nature 428: 84-88). Brennecke et al. 2003 (Cell 113: 25-36) and Mansfield et al. 2004 (Nature Genetics 36: 1079-83) report on an alternative method in which reporter transgenes, so-called sensors, are designed and generated to detect the presence of a given miRNA in an embryo. Each sensor contains a constitutively expressed reporter gene (e.g. lacZ or green fluorescent protein) harbouring miRNA target sites in its 3′-UTR. Thus, in cells that lack the miRNA in question, the transgene RNA is stable allowing detection of the reporter, whereas cells expressing the miRNA, the sensor mRNA is targeted for degradation by the RNAi pathway. Although sensitive, this approach is time-consuming since it requires generation of the expression constructs and transgenes. Furthermore, the sensor-based technique detects the spatiotemporal miRNA expression patterns via an indirect method as opposed to direct in situ hybridization of the mature miRNAs.

The large number of miRNAs along with their small size makes it difficult to create loss-of-function mutants for functional genomics analyses. Another potential problem is that many miRNA genes are present in several copies per genome occurring in different loci, which makes it even more difficult to obtain mutant phenotypes. Boutla et al. 2003 (Nucleic Acids Research 31: 4973-4980) describe the use of DNA antisense oligonucleotides complementary to 11 different miRNAs in Drosophila as well as their use to inactivate the miRNAs by injecting the DNA oligonucleotides into fly emryos. Of the 11 DNA antisense oligonucleotides, only 4 constructs showed severe interference with normal development, while the remaining 7 oligonucleotides didn't show any phenotypes presumably due to their inability to inhibit the miRNA in question. Thus, the succes rate for using DNA antisense oligonucleotides to inhibit miRNA function would most likely be too low to allow functional analyses of miRNAs on a larger, genomic scale. An alternative approach to this has been reported by Hutvagner et al. 2004 (PLoS Biology 2: 1-11), in which 2′-O-methyl antisense oligonucleotides could be used as potent and irreversible inhibitors of siRNA and miRNA function in vitro and in vivo in Drosophila and C. elegans, thereby inducing a loss-of-function phenotype. A drawback of this method is the need of high 2′-O-methyl oligonucleotide concentrations (100 micromolar) in transfection and injection experiments, which may be toxic to the animal.

In conclusion, the biggest challenge in detection, quantitation and functional analysis of the mature miRNAs as well as siRNAs using currently available methods is their small size of the of 19-25 nt and often low level of expression

RNA Editing and Alternative Splicing

RNA editing is used to describe any specific change in the primary sequence of an RNA molecule, excluding other mechanistically defined processes such as alternative splicing or polyadenylation. RNA alterations due to editing fall into two broad categories, depending on whether the change happens at the base or nucleotide level (Gott 2003, C. R. Biologies 326 901-908). RNA editing is quite widespread, occurring in mammals, viruses, marsupials, plants, flies, frogs, worms, squid, fungi, slime molds, dinoflagellates, kinetoplastid protozoa, and other unicellular eukaryotes. The current list most likely represents only the tip of the iceberg; based on the distribution of homologues of known editing enzymes, as RNA editing almost certainly occurs in many other species, including all metazoa. Since RNA editing can be regulated in a developmental or tissue-specific manner, it is likely to play a significant role in the etiology of human disease (Goff 2003, C. R. Biologies 326 901-908).

A common feature for eukaryotic genes is that they are composed of protein-encoding exons and introns. Introns are characterized by being excised from the pre-mRNA molecule in RNA splicing, as the sequences on each side of the intron are spliced together. RNA splicing not only provides functional mRNA, but is also responsible for generating additional diversity. This phenomenon is called alternative splicing, which results in the production of different mRNAs from the same gene. The mRNAs that represent isoforms arising from a single gene can differ by the use of alternative exons or retention of an intron that disrupts two exons. This process often leads to different protein products that may have related or drastically different, even antagonistic, cellular functions. There is increasing evidence indicating that alternative splicing is very widespread (Croft et al. Nature Genetics, 2000). Recent studies have revealed that at least 80% of the roughly 35,000 genes in the human genome are alternatively spliced (Kampa et al. 2004, Genome Research 14: 331-342). Clearly, by combining different types of modifications and thus generating different possible combinations of transcripts of different genes, alternative splicing together with RNA editing are potent mechanisms for generating protein diversity. Analysis of the alternative splice variants and RNA editing, in tum, represents a novel approach to functional genomics, disease diagnostics and pharmacogenomics.

Misplaced Control of Alternative Splicing as a Causative Agent for Human Disease

The detection of the detailed structure of the transcriptional output is an important goal for molecular characterization of a cell or tissue. Without the ability to detect and quantify the splice variants present in one tissue, the transcript content or the protein content cannot be described accurately. Molecular medical research shows that many cancers result in altered levels of splice variants, so an accurate method to detect and quantify these transcripts is required. Mutations that produce an aberrant splice form can also be the primary cause of such severe diseases such as spinal muscular dystrophy and cystic fibrosis.

Much of the study of human disease, indeed much of genetics is based upon the study of a few model organisms. The evolutionary stability of alternative splicing patterns and the degree to which splicing changes according to mutations and environmental and cellular conditions influence the relevance of these model systems. At present, there is little understanding of the rates at which alternative splicing patterns or RNA editing change, and the factors influencing these rates.

Previously, other analysis methods have been performed with the aim of detecting either splicing of RNA transcripts per se in yeast, or of detecting putative exon skipping splicing events in rat tissues, but neither of these approaches had sufficient resolution to estimate quantities of splice variants, a factor that could be essential to an understanding of the changes in cell life cycle and disease. Thus, improved methods are needed for nucleic acid hybridization and quantitation.

Antisense Transcription in Eukaryotes

RNA-mediated gene regulation is widespread in higher eukaryotes and complex genetic phenomena like RNA interference, co-suppression, transgene silencing, imprinting, methylation, and possibly position-effect variegation and transvection, all involve intersecting pathways based on or connected to RNA signalling (Mattick 2001; EMBO reports 2, 11: 986-991). Recent studies indicate that antisense transcription is a very common phenomenon in the mouse and human genomes (Okazaki et al. 2002; Nature 420: 563-573; Yelin et al. 2003, Nature Biotechnol.). Thus, antisense modulation of gene expression in eukaryotic cells, e.g. human cells appear to be a common regulatory mechanism.

### SUMMARY OF THE INVENTION

The challenges of establishing genome function and understanding the layers of information hidden in the complex transcriptomes of higher eukaryotes call for novel, improved technologies for detection and analysis of non-coding RNA and protein-coding RNA molecules in complex nucleic acid samples. Thus, it would be highly desirable to be able to detect and analyse the expression of mature microRNAs, siRNAs, RNA-edited transcripts as well as highly homologous splice variants in the transcriptomes of eukaryotes using methods based on specific and sensitive oligonucleotide detection probes.

The present invention solves the current problems faced by conventional approaches used in detection and analysis of mature miRNAs, their target mRNAs as well as siRNAs as outlined above by providing a method for the design, synthesis and use of novel oligonucleotide compositions and probe sequences with improved sensitivity and high sequence specificity for RNA target sequences, such as mature miRNAs and siRNAs so that they are unlikely to detect a random RNA target molecule. Such oligonucleotide probes comprise a recognition sequence complementary to the RNA target sequence, which said recognition sequence is substituted with high-affinity nucleotide analogues, e.g. LNA, to increase the sensitivity and specificity of conventional oligonucleotides, such as DNA oligonucleotides, for hybridization to short target sequences, e.g. mature miRNAs, stem-loop precursor miRNAs, siRNAs or other non-coding RNAS as well as miRNA binding sites in their cognate mRNA targets, mRNAs, mRNA splice variants, RNA-edited mRNAs and antisense RNAs.

In one aspect, the invention features a nucleotide probe including a plurality of LNA monomers that hybridizes to a miRNA. Desirably, two of the plurality of LNA monomers are disposed 3 or 4 nucleotides apart, or a combination thereof. In other embodiments, each LNA monomer in a probe is spaced 3 or 4 nucleotides from the closest LNA monomer. When LNA monomers are spaced apart, only naturally-occurring nucleotides may be disposed between the LNA monomers. In another embodiment, two, three, four, or more LNA monomers are disposed adjacent to one another. The adjacent LNA monomers may be disposed at the 3′ or 5′ end or so that one of the LNA monomers hybridizes to the center of the miRNA. In other embodiments, the prove includes none or at most one mismatched base, deletion, or addition. Desirably, the probe hybridizes to the miRNA under stringent conditions. Or high stringency conditions. In certain embodiments, the melting point of the duplex formed between the probe and the miRNA is at least 1° C. higher, e.g., at least 5° C., than the melting point of the duplex formed between the miRNA and a nucleic acid sequence not comprising a LNA monomer or any modified backbone. The probe may include at least 70% DNA; at least 10% LNA units; and/or at most 30% LNA units. In addition, the probe may be at least 8 nucleotides long and at most 30 nucleotides long. The probe may further include a 5′ or 3′ amino group and/or a 5′ or 3′ label, e.g., a fluorescent (such as fluorescein) or radioactive label. Other potential modifications of probes are described herein. In other embodiments, the probe when hydridized to the miRNA provides a substrate for RNase H; alternatively, the probe when hybridized to the miRNA may not provide a substrate for RNase H. Preferably, the probes of the invention exhibit increases binding affinity for the target sequence by at least two-fold compared to probes of the same sequence without the modification, under the same conditions for hybridization or stringent hybridization conditions.

The invention further features a method of creating a nucleotide duplex by providing a miRNA; and contacting the miRNA with a probe of the invention that hybridizes to the miRNA. The invention also features a method of inhibiting the biological activity of a miRNA by providing the miRNA; and contacting the miRNA with a probe of the invention that hybridizes to said miRNA, thereby inhibiting the biological activity of the miRNA. In addition, the invention features a method of determining the biological activity of a miRNA by providing the miRNA; contacting the miRNA with a probe of the invention that hybridizes to the miRNA; and assaying the biological activity. Any method of the invention may involve contacting a probe with miRNA that is endogenously or exogenously produced. Such contacting may occur in vitro or in vivo or within or without a cell, which may or may not naturally express the miRNA.

In another aspect, the invention feauters a kit including a probe of the invention and packaging and/or labeling indicative of the miRNA to which the probe hybridizes and conditions under which the hybridization occurs.

Exemplary miRNAs are described herein and are known in the art, e.g., in U.S. 2005/0182005; WO 2005/013901, and the miRBase Sequence Database (D140-D144 Nucleic Acids Research, 2006, Vol. 34, Database issue), each of which is hereby incorporated by reference.

The invention also features probes, as described herein, in combination with a pharmaceutically acceptable carrier. Such carriers are known in the art.

Also, discussed primarily with respect to miRNA, LNA containing probes, polynucleotides, and oligonucleotides are broadly applicable to other antisense uses, as described herein.

The present invention provides the design and development of novel oligonucleotide compositions and probe sequences for accurate, highly sensitive and specific detection and functional analysis of miRNAs, their target mRNAs and siRNA transcripts.

The present invention enables discrimination between mRNA splice variants as well as RNA-edited transcripts and detects each variant in a nucleic acid sample, such as a sample derived from a patient in e.g. addressing the spatiotemporal expression patterns by RNA in situ hybridization.

The present invention provides a method for detection and functional analysis of non-coding antisense RNAs, as well as a method for detecting the overlapping regions between sense-antisense transcriptional units.

The invention features a method of designing the detection probe sequences by selecting optimal substitution patterns for the high-affinity analogues, e.g. LNAs for the detection probes. This method involves (a) substituting the detection probe sequence with the high affinity analogue LNA in chimeric LNA-DNA oligonucleotides using regular spacing between the LNA substitutions, e.g. at every second nucleotide position, every third nucleotide position, or every fourth nucleotide position, in order to promote the A-type duplex geometry between the substituted detection probe and its complementary RNA target; with the said LNA monomer substitutions spiked in all the possible phases in the probe sequence with an unsubstituted monomer at the 5′-end position and 3′-end position in all the substituted designs; (b) determining the ability of the designed detection probes with different regular substitution patterns to self-anneal; and (c) determining the melting temperature of the substituted probes sequences of the invention, and (d) selecting the probe sequences with the highest melting temperatures and lowest self-complementarity score, i.e. lowest ability to self-anneal are selected. In another aspect of the invention

In another aspect the invention features a method of designing the detection probe sequences by selecting optimal substitution patterns for the LNAs, which said method involves substituting the detection probe sequence with the high affinity analogue LNA in chimeric LNA-DNA oligonucleotides using irregular spacing between the LNA monomers and selecting the probe sequences with the highest melting temperatures and lowest self-complementarity score. In yet another aspect the invention features a computer code for a preferred software program of the invention for the design and selection of the said substituted detection probe sequences.

In another aspect the invention features detection probe sequences containing a ligand. Such ligand-containing detection probes of the invention are useful for isolating target RNA molecules from complex nucleoc acid mixtures, such as miRNAs, their cognate target mRNAs and siRNAs. Ligands comprise biotin and functional groups such as: aromatic groups (such as benzene, pyridine, naphthalene, anthracene, and phenanthrene), heteroaromatic groups (such as thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicar-bazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C1-C20 alkyl groups optionally interrupted or terminated with one or more heteroatoms such as oxygen atoms, nitrogen atoms, and/or sulphur atoms, optionally containing aromatic or mono/polyunsaturated hydrocarbons, polyoxyethylene such as polyethylene glycol, oligo/polyamides such as poly-α-alanine, polyglycine, polylysine, peptides, oligo/polysaccharides, oligo/polyphosphates, toxins, antibiotics, cell poisons, and steroids, and also affinity ligands, i.e. functional groups or biomolecules that have a specific affinity for sites on particular proteins, antibodies, poly- and oligosaccharides, and other biomolecules.

In another aspect the invention features detection probe sequences, which sequences have been further modified by Selectively Binding Complementary (SBC) nucleobases, i.e. modified nucleobases that can make stable hydrogen bonds to their complementary nucleobases, but are unable to make stable hydrogen bonds to other SBC nucleobases. Such SBC monomer substitutions are especially useful when highly self-complementary detection probe sequences are employed. As an example, the SBC nucleobase A′, can make a stable hydrogen bonded pair with its complementary unmodified nucleobase, T. Likewise, the SBC nucleobase T′ can make a stable hydrogen bonded pair with its complementary unmodified nucleobase, A. However, the SBC nucleobases A′ and T′ will form an unstable hydrogen bonded pair as compared to the base pairs A′-T and A-T′. Likewise, a SBC nucleobase of C is designated C′ and can make a stable hydrogen bonded pair with its complementary unmodified nucleobase G, and a SBC nucleobase of G is designated G′ and can make a stable hydrogen bonded pair with its complementary unmodified nucleobase C, yet C′ and G′ will form an unstable hydrogen bonded pair as compared to the base pairs C′-G and C-G′. A stable hydrogen bonded pair is obtained when 2 or more hydrogen bonds are formed e.g. the pair between A′ and T, A and T′, C and G′, and C′ and G. An unstable hydrogen bonded pair is obtained when 1 or no hydrogen bonds is formed e.g. the pair between A′ and T′, and C′ and G′. Especially interesting SBC nucleobases are 2,6-diaminopurine (A′, also called D) together with 2-thio-uracil (U′, also called 2SU)(2-thio4-oxo-pyrimidine) and 2-thio-thymine (T′, also called 2ST)(2-thio4-oxo-5-methyl-pyrimidine).

In another aspect the detection probe sequences of the invention are covalently bonded to a solid support by reaction of a nucleoside phosphoramidite with an activated solid support, and subsequent reaction of a nucleoside phosphoramide with an activated nucleotide or nucleic acid bound to the solid support. In some embodiments, the solid support or the detection probe sequences bound to the solid support are activated by illumination, a photogenerated acid, or electric current. In other embodiments the detection probe sequences contain a spacer, e.g. a randomized nucleotide sequence or a non-base sequence, such as hexaethylene glycol, between the reactive group and the recognition sequence. Such covalently bonded detection probe sequence populations are highly useful for large-scale detection and expression profiling of mature miRNAs, stem-loop precursor miRNAs, siRNAs and other non-coding RNAs.

The present oligonucleotide compositions and detection probe sequences of invention are highly useful and applicable for detection of individual small RNA molecules in complex mixtures composed of hundreds of thousands of different nucleic acids, such as detecting mature miRNAs, their target mRNAs or siRNAs, by Northern blot analysis or for addressing the spatiotemporal expression patterns of miRNAs, siRNAs or other non-coding RNAs as well as mRNAs by in situ hybridization in whole-mount embryos, whole-mount animals or plants or tissue sections of plants or animals, such as human, mouse, rat, zebrafish, Caenorhabditis elegans, Drosophila melanogaster, Arabidopsis thaliana, rice and maize. The present oligonucleotide compositions and detection probe sequences of the invention are furthermore highly useful and applicable for large-scale and genome-wide expression profiling of mature miRNAs, siRNAs or other non-coding RNAs in animals and plants by oligonucleotide microarrays. The present oligonucleotide compositions and detection probe sequences are furthermore highly useful in functional analysis of miRNAs, siRNAs or other non-coding RNAs in vitro and in vivo in plants or animals, such as human, mouse, rat, zebrafish, Caenorhabditis elegans, Drosophila melanogaster, Arabidopsis thaliana, rice and maize, by inhibiting their mode of action, e.g. the binding of mature miRNAs to their cognate target mRNAs. The oligonucleotide compositions and detection probe sequences of invention are also applicable to detecting, testing, diagnosing or quantifying miRNAs, siRNAs, other non-coding RNAs, RNA-edited transcripts or alternative mRNA splice variants implicated in or connected to human disease in complex human nucleic acid samples, e.g. from cancer patients.

Other features and advantages of the invention will be apparent from the following description, the figures, and the claims.

### BRIEF DESCRIPTION OF THE DRAWINGS

FIG. 1: Expression of selected miRNAs during muscle differentiation. Northern blot of ES or C2C12 cells, either proliferating or differentiating. U6 was used as a loading control.

FIG. 2: Mir181 is expressed in muscle. A) time course of miR-181 upregulation and muscle marker expression in ES or C2C12 cells; upper panel: northern blot (NB), P: 76 nt precursor, m:mature microRNA, Br: brain (positive control); lower panel: western blot WB), MCK: muscle creatine kinase, α-tub: α-tubulin (used as a loading control). B) time course of miR-181 expression during cardiotoxin-induced regeneration of tibialis anterior (TA) muscle of 6-week-old Balb/c mice in vivo; ES cells were used as a negative, and embryonic bodies (EB) as a positive control. C) miR-181 expression in resting muscle or during cardiotoxin-induced regeneration of tibialis anterior, as detected by in situ hybridization: transversal slices of muscles were probed for miR181(FITC), and nuclei were couterstained (DAPI).

FIG. 3: Inhibition of miRNA by LNA/DNA antisense oligonucleotides. A) sequences of oligonucleotides; lower case: non-modified nucleotides; upper case: locked nucleotides; wt: wild type; scr: scrambled. B) C2C12 cells were transfected with anti-miR-125b oligonucleotides as indicated, and miR-125b was analyzed by northern blot as in FIG. 1. C) in vitro analysis of the wt LNA/miRNA complex: a fixed amount of radiolabelled miRNA was incubated with increasing amounts of LNA at indicated ratios, and the mixture was resolved on a denaturing gel; MW: molecular weight marker, number on the right indicate the size of the ladder's component (in nucleotides). D) C2C12 cells were transfected with the indicated LNAs and analyzed by northern; a smear above the position of the miRNA that might correspond to melting LNA/miRNA duplexes is marked by a star; E) dose curve and F) time course analysis of inhibition; C2C12 cells were transfected with indicated doses of the anti-miR-125b wt 8 LNA and analyzed by northern blot.

FIG. 4: Inhibition of miR-181 affects myoblastic differentiation. C2C12 cells were transfected with indicated doses of anti-miR-181a antisense LNA or siRNA (sequences in panel A; L: LNA; S: siRNA; mutations are underlined); MHC expression was analyzed by immunofluorescence (B; DAPI stains of the fields are also shown), MCK was detected by western blotting (C), and miR-181 was detected by northern blotting (D); a smear above the position of the precursor that might correspond to melting LNA/precursor duplexes is marked by a star.

FIG. 5: Rescue experiments. A: sequence of the miR-181 synthetic oligonucleotide. B and C: Cells were transfected with the LNAs (L/wt: wild type LNA, L/mut: mutant LNA), alone or along with a synthetic RNA oligonucleotide corresponding to miR181 (Lwt+R); B) photomicrographs of transfected cells; C) western blot analysis of MCK expression.

FIG. 6: MiR181 and Hox-a11 are in the same pathway. A) Hox-a11 expression in resting muscle or during cardiotoxin-induced regeneration of tibialis anterior, as detected by in situ immunofluorescence: transversal slices of muscle were labelled with anti-Hox26 a11 antibodies, and nuclei were counterstained (DAPI). B) Inhibition of Hox-a11 expression using an siRNA: C2C12 cells were transfected with indicated synthetic siRNAs and kept in proliferation medium for 24h; extracts were analyzed by western blot using anti-Hoxa11 antibodies. C) MiR181 and Hox-a11 belong to the same genetic pathway: C2C12 cells were transfected with the indicated siRNAs and LNAs, and put in differentiation medium for 2 days; MCK expression was monitored by western blot. wt+R: control cells transfected with the anti-miR181 LNA and the synthetic miRNA.

FIG. 7: Supplemental data.

FIG. 8: miR-181 is expressed in muscle. (a) Time-course of miR-181 induction and muscle marker expression in embryonic stem (ES) or C2C12 cells. P, miRNA precursor; M, mature miRNA; Br, brain (positive control). α-Tubulin was used as a loading control. (b) The a and b isoforms of miR-181 are co-expressed in differentiating myoblasts. Synthetic RNA oligonucleotides corresponding to the miR-181 isoforms were analysed by northern blot together with RNA extracted from proliferating (P) or differentiating (D, differentiation was for 3 d) cells, using LNA modified probes complementary to miR-181a or miR-181b as indicated; on the left are the positions of a 10 nucleotide RNA ladder; bp, base pairs. Note that the two gels have migrated independently. (c) Time course of miR-181 expression during cardiotoxin-induced regeneration of the tibialis anterior muscle of 6-week-old Balb/c mice in vivo (cardiotoxin injected at day 0); (d) Cells expressing miR-181 are differentiating muscle cells. Cross-sections of tibialis anterior muscles at day 5 of cardiotoxin-induced regeneration were submitted to immuno- FISH using antibodies against embryonic MHC (eMHC) with TRITC-labelled secondary antibodies, and a DIG-labelled oligonucleotide probe complementary to miR-181 with FITC-labelled secondary antibodies; sections were also counterstained with DAPI. Magnification: ×200. Scale bar represents 25 μm. (e) Higher magnification (×630) of the fields shown in d. Scale bar represents 100 μm. (f) Time course of eMHC and miR-181 expression during regeneration (0: injection of cardiotoxin); time points in the shaded area were not tested.

FIG. 9: Inhibition of miR-181 affects myoblast differentiation. (a, b) miR-181 LNA interferes with miR-181 detection. Sequences are shown in a. Mutations are underlined. miR-181 was detected by northern blotting (b). U6 was used as a loading control; P, precursor; M, mature miR-181. (c) miR-181 LNA interferes with miR-181 function. C2C12 cells were transfected with firefly luciferase reporter constructs harbouring a sequence complementary to miR-181, or a mutated version of this sequence (mutations as in a) in their 3′UTR, together with a Renilla construct to monitor transfection efficiency, and placed under differentiation conditions; luciferase was measured after 2 d. Graphs present the firefly:renilla ratios standardized with respect to the control sample to eliminate variations due to the construct itself. (d, e) C2C12 cells were transfected with the anti-miR-181a antisense LNA (50 nM), either wild-type or mutated; MHC expression was analysed by immunofluorescence microscopy (DAPI staining also shown), and MCK was detected by western blotting (e). Scale bar represents 100 μm. (f) Rescue experiments. C2C12 cells were transfected with the control (con) or miR-181 antisense (miR-181) LNA oligonucleotide (50 nM); after 24 h cells were transfected again with synthetic miR-181a (+; 75 nM) or a control double-stranded RNA sequence (−) and placed under differentiation conditions as described on the left; at day 3 cell extracts were analysed by western blot. (g, h) Inhibition of miR-181 affects MyoD and myogenin induction. C2C12 cells were transfected with the indicated LNAs (mut, mutant LNA; WT, wild-type miR-181 LNA) and placed under differentiation conditions. MyoD (f) or myogenin (g) expression was monitored by western blotting at the indicated times. β-actin and α-tubulin are shown as loading controls.

FIG. 10: Hox-A11 expression pattem in muscle. (a) Hox-A11 expression in resting and regenerating tibialis anterior muscle, as detected by in situ immunofluorescence microscopy. Cross-sections were labelled with anti-Hox- A11 antibodies (Hox-A11), and nuclei were counterstained (DAPI). Two areas are shown; one corresponding to resting muscle surrounding the regeneration area (resting area) and one corresponding to the regeneration area itself (regenerating area). Scale bar represents 50 μm. (b, c) Hox-A11 expression during myoblast cell differentiation in vitro. Extracts of differentiating C2C12 cells were analysed by western blot at the indicated times, using anti-Hox-A11 or anti-MCK antibodies (b) or by quantitative real-time PCR using primers for Hox-A11 or MCK detection (c). RNA was quantified after standardization with 36B4 mRNA (used as an internal control), and is presented as the percentage of maximal value for each of the Hox-A11 and MCK RNAs.

FIG. 11: Hox-A11 is a target for miR-181. (a) Hox-A11 protein is sensitive to ectopic miR-181 expression. Proliferating C2C12 cells were transfected with a double-stranded synthetic RNA oligonucleotide corresponding to miR-181a (+), or with a control sequence (−), and extracts were analysed by western blot using anti-Hox-A11 antibodies. (b) Inhibition of miR-181 by a miR-181 antisense LNA upregulates Hox-A11 protein. C2C12 cells were transfected with an anti-miR-181 LNA or a control LNA as indicated, and placed under differentiation conditions. Extracts were analysed at indicated times, using an anti-Hox-A11 antibody, or an anti-α-tubulin as a control. (c, d) Hox-A11 predicted target sequence confers sensitivity to miR-181. Firefly luciferase constructs harbouring four tandem repeats of the Hox-A11 target sequences (either wild-type or mutant sequences as shown in c) in their 3′UTR were transfected into HeLa cells together with synthetic double-stranded oligonucleotides corresponding to miR- 181a, miR-181b1, miR-181b2, or miR196 (a miRNA that controls the expression of other Hox proteins and in particular Hox-B8 (ref. 14 of Example 13), and with a Renilla reporter construct as a control. Luciferases were measured 24 h later. The ratios between firefly and Renilla are shown; mean ±s. d., n =3. (e, f) Hox-A11 is an important target of miR-181. Inhibition of Hox-A11 expression using an siRNA (e). C2C12 cells were transfected with the indicated synthetic siRNAs and kept in proliferation medium for 24 h; extracts were analysed by western blot using anti-Hox-A 11 antibodies. (f, g) Hox-A11 downregulation suppresses the phenotype induced by miR- 181 inhibition. C2C12 cells were transfected with an siRNA against Hox- A 11 (Hox) or a control scrambled sequence (C) along with mutant (Mut) or wild-type (WT) miR-181 antisense LNAs as indicated, and placed in differentiation medium for 2 d; MCK, MHC (f) and Hox-A11 (g) expression was monitored by western blot. WT+R: control cells transfected with equimolar amounts of the anti-miR-181 LNA and the synthetic miRNA so that the effect of the LNA was abolished.

FIG. 12: miR-181 pathway in terminally differentiating myoblasts. On differentiation, miR-181 is upregulated, resulting in downregulation of Hox-A11 and in the release of MyoD expression. As a result, myogenin and muscle marker proteins (MHC, MCK) are upregulated.

FIG. 13: miR-181c is not expressed in C2C12 cells: Equal amounts of synthetic RNA oligonucleotides (synthetic miR-181), or extracts from proliferating (P) or differentiating (D) C2C12 cells, or else a mixture of proliferating cell extracts and synthetic miR181 RNAs (P+a, b or c) were analyzed by Northern blot, using an LNA probe (Exiqon) complementary to miR-181c. The probe anneals to the a and b iso-forms although far less than to the c, and detects a product in cell extracts; however, analysis of the size indicates that no band migrating at the position of synthetic miR181c is apparent in the extracts; this is not due to the conditions of migration, since synthetic miR-181c migrates at the expected position and is recognized by the c probe even when mixed with extracts from cells (P+c); note that the right panel is a much stronger exposure than the left panel.

FIG. 14a: anti-miR-181a LNA inhibits miR-181a and miR-181b. C2C12 cells were transfected with control (c) or anti-miR-181 (181) LNA and analyzed by Northern blot as described in FIG. 9, using probes recognizing preferentially mir-181a or miR181b as indicated (see FIG. 8b).

FIG. 14b: Synthetic miRNAs transfected into C2C12 cells. Proliferating C2C12 cells were transfected with the double-stranded synthetic oligonucleotides (50 nM) shown in the upper panel (mismatches are in bold; miR-181a-1: a fully annealed doubled-stranded sequence: miR-181a-2: a double-stranded sequence destabilized to favour incorporation of the miRNA strand into protein complexes: miR181a-3, a double stranded sequence mimicking the predicted product of precursor processing by DICER). Northern blot analysis of transfected cells showed that the last design was the most efficient with regard to the level of intracellular miRNA (lower panel; 1, 2, 3: miR-181a-1, miR-181a-2 and miR- 181a-3), and was used in subsequent experiments.

FIG. 14c: p21 is down-regulated in miR-181-depleted cells. C2C12 cells were transfected with the control (c) or wt LNAs as in FIG. 9g and f,and p21 expression was monitored by western blot after 24 h.

FIG. 15a: HoxA11 mRNA is not affected by anti-miR-181 LNA: C2C12 cells were transfected with 50 nM of LNA, either control (con) or anti-miR-181 (miR-181) or mock transfected (−), placed in differentiation medium for 2 days (dif) or kept in proliferation medium (prol), and analyzed by real time RT-PCR for HoxA11 and 36B4 mRNA. Shown are the ratios between HoxA11 and 36B4.

FIG. 15b: MiR181 a and b do not synergize for HoxA11 target inhibition: HeLa cells were transfected as in FIG. 11d with either 50 nM of synthetic double-stranded miRNA (c: miR-196, a: miR-181a as in FIG. 9e, b2:miR-181 b2), or a mixture of two, as indicated, at 25 nM each. Shown is the ratio between firefly and Renilla luciferases (bars indicate standard deviations).

FIG. 15c: Several mIRNAs predicted to bind to HoxA11 mRNA are upregulated in differentiating myoblasts. RNA from C2C12 cells, either proliferating (P) or after 3 days of differentiation (D), were analyzed by Northern blot as described in FIG. 8, using probes complementary to miR-23a,miR-188, miR-339 or miR- 30b; Brain (Br) is shown as a positive control and U6 as a loading control.

FIG. 15d: Down-regulation of HoxA11 or ectopic expression of miR-181 do not induce differentiation of proliferating C2C12 cells. C2CC12 cells were transfected with a control siRNA (c) an siRNA against Hox-A11 (Hox) or synthetic double-stranded miR181a (181) as in FIG. 11E ), kept in proliferation medium (prol) or placed under differentiation conditions (dif) and analyzed by western blot 36h later (for myogenin) or 48h later (MCK).

FIG. 16: Efficient uptake of fluorochrome-labelled miRCURY™ knockdown probes (LNA probes from Exiqon) into human K562 cells.

FIG. 17: The effective knockdown effect of miRCURY™ antisense molecules.

FIG. 18: Knockdown of dme-bantam miRNA in Drosophila KC167 cells by miRCURY™ antisense.

### DEFINITIONS

In the present context “ligand” means something that binds. Ligands comprise biotin and functional groups such as: aromatic groups (such as benzene, pyridine, naphthalene, anthracene, and phenanthrene), heteroaromatic groups (such as thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C₁-C₂₀ alkyl groups optionally interrupted or terminated with one or more heteroatoms such as oxygen atoms, nitrogen atoms, and/or sulphur atoms, optionally containing aromatic or mono/polyunsaturated hydrocarbons, polyoxyethylene such as polyethylene glycol, oligo/polyamides such as poly-β-alanine, polyglycine, polylysine, peptides, oligo/polysaccharides, oligo/polyphosphates, toxins, antibiotics, cell poisons, and steroids, and also “affinity ligands”, i.e. functional groups or biomolecules that have a specific affinity for sites on particular proteins, antibodies, poly- and oligosaccharides, and other biomolecules.

The singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

“Transcriptome” refers to the complete collection of transcriptional units of the genome of any species. In addition to protein-coding mRNAs, it also represents non-coding RNAs, such as small nucleolar RNAs, siRNAs, microRNAs and antisense RNAs, which comprise important structural and regulatory roles in the cell.

“Sample” refers to a sample of cells, or tissue or fluid isolated from an organism or organisms, including but not limited to, for example, skin, plasma, serum, spinal fluid, lymph fluid, synovial fluid, urine, tears, blood cells, organs, tumours, and also to samples of in vitro cell culture constituents (including but not limited to conditioned medium resulting from the growth of cells in cell culture medium, recombinant cells and cell components).

An “organism” refers to a living entity, including but not limited to, for example, human, mouse, rat, Drosophila, C. elegans, yeast, Arabidopsis thaliana, maize, rice, zebra fish, primates, domestic animals, etc.

The terms “detection probes” or “detection probe” or “detection probe sequence” refer to an oligonucleotide, which oligonucleotide comprises a recognition sequence complementary to a RNA target sequence, which said recognition sequence is substituted with high-affinity nucleotide analogues, e.g. LNA, to increase the sensitivity and specificity of conventional oligonucleotides, such as DNA oligonucleotides, for hybridization to short target sequences, e.g. mature miRNAs, stem-loop precursor miRNAs, pri-miRNAs, siRNAs or other non-coding RNAs as well as miRNA binding sites in their cognate mRNA targets, mRNAs, mRNA splice variants, RNA-edited mRNAs and antisense RNAs.

The terms “miRNA” and “microRNA” refer to 19-25 nt, e.g., 21-25, non-coding RNAs derived from endogenous genes. They are processed from longer (ca 75 nt) hairpin-like precursors termed pre-miRNAs. MicroRNAs assemble in complexes termed miRNPs and recognize their targets by antisense complementarity. If the microRNAs match 100% their target, i.e. the complementarity is complete, the target mRNA is cleaved, and the miRNA acts like a siRNA. If the match is incomplete, i.e. the complementarity is partial, then the translation of the target mRNA is blocked.

The terms “Small interfering RNAs” or “siRNAs” refer to 21-25 nt RNAs derived from processing of linear double-stranded RNA. siRNAs assemble in complexes termed RISC (RNA-induced silencing complex) and target homologous RNA sequences for endonucleolytic cleavage. Synthetic siRNAs also recruit RISCs and are capable of cleaving homologous RNA sequences

The term “RNA interference” (RNAi) refers to a phenomenon where double-stranded RNA homologous to a target mRNA leads to degradation of the targeted mRNA. More broadly defined as degradation of target mRNAs by homologous siRNAs.

The term “recognition sequence” refers to a nucleotide sequence that is complementary to a region within the target nucleotide sequence essential for sequence-specific hybridization between the target nucleotide sequence and the recognition sequence.

The term “label” as used herein refers to any atom or molecule which can be used to provide a detectable (preferably quantifiable) signal, and which can be attached to a nucleic acid or protein. Labels may provide signals detectable by fluorescence, radioactivity, colorimetric, X-ray diffraction or absorption, magnetism, enzymatic activity, and the like.

As used herein, the terms “nucleic acid”, “polynucleotide” and “oligonucleotide” refer to primers, probes, oligomer fragments to be detected, oligomer controls and unlabelled blocking oligomers and shall be generic to polydeoxyribonucleotides (containing 2-deoxy-D-ribose), to polyribonucleotides (containing D-ribose), and to any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA. The oligonucleotide is comprised of a sequence of approximately at least 3 nucleotides, preferably at least about 6 nucleotides, and more preferably at least about 8-30 nucleotides corresponding to a region of the designated target nucleotide sequence. “Corresponding” means identical to or complementary to the designated sequence. The oligonucleotide is not necessarily physically derived from any existing or natural sequence but may be generated in any manner, including chemical synthesis, DNA replication, reverse transcription or a combination thereof.

The terms “oligonucleotide” or “nucleic acid” intend a polynucleotide of genomic DNA or RNA, cDNA, semi synthetic, or synthetic origin which, by virtue of its origin or manipulation: (1) is not associated with all or a portion of the polynucleotide with which it is associated in nature; and/or (2) is linked to a polynucleotide other than that to which it is linked in nature; and (3) is not found in nature. Because mononucleotides are reacted to make oligonucleotides in a manner such that the 5′-phosphate of one mononucleotide pentose ring is attached to the 3′ oxygen of its neighbour in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have a 5′ and 3′ ends. When two different, non-overlapping oligonucleotides anneal to different regions of the same linear complementary nucleic acid sequence, the 3′ end of one oligonucleotide points toward the 5′ end of the other; the former may be called the “upstream” oligonucleotide and the latter the “downstream” oligonucleotide.

By the term “SBC nucleobases” is meant “Selective Binding Complementary” nucleobases, i.e. modified nucleobases that can make stable hydrogen bonds to their complementary nucleobases, but are unable to make stable hydrogen bonds to other SBC nucleobases. As an example, the SBC nucleobase A′, can make a stable hydrogen bonded pair with its complementary unmodified nucleobase, T. Like-wise, the SBC nucleobase T′ can make a stable hydrogen bonded pair with its complementary unmodified nucleobase, A. However, the SBC nucleobases A′ and T′ will form an unstable hydrogen bonded pair as compared to the base pairs A′-T and A-T′. Likewise, a SBC nucleobase of C is designated C′ and can make a stable hydrogen bonded pair with its complementary unmodified nucleobase G, and a SBC nucleobase of G is designated G′ and can make a stable hydrogen bonded pair with its complementary unmodified nucleobase C, yet C′ and G′ will form an unstable hydrogen bonded pair as compared to the base pairs C′-G and C-G′. A stable hydrogen bonded pair is obtained when 2 or more hydrogen bonds are formed e.g. the pair between A′ and T, A and T′, C and G′, and C′ and G. An unstable hydrogen bonded pair is obtained when 1 or no hydrogen bonds is formed e.g. the pair between A′ and T′, and C′ and G′. Especially interesting SBC nucleobases are 2,6-diaminopurine (A′, also called D) together with 2-thio-uracil (U′, also called ^{2S}U)(2-thio4-oxo-pyrimidine) and 2-thio-thymine (T′, also called ^{2S}T)(2-thio4-oxo-5-methyl-pyrimidine).

“SBC LNA oligomer” refers to a “LNA oligomer” containing at least one LNA monomer where the nucleobase is a “SBC nucleobase”. By “LNA monomer with an SBC nucleobase” is meant a “SBC LNA monomer”. Generally speaking SBC LNA oligomers include oligomers that besides the SBC LNA monomer(s) contain other modified or naturally occurring nucleotides or nucleosides. By “SBC monomer” is meant a non-LNA monomer with a SBC nucleobase. By “isosequential oligonucleotide” is meant an oligonucleotide with the same sequence in a Watson-Crick sense as the corresponding modified oligonucleotide e.g. the sequences agTtcATg is equal to agTscD^{2S}Ug where s is equal to the SBC DNA monomer 2-thio-t or 2-thio-u, D is equal to the SBC LNA monomer LNA-D and ^{2S}U is equal to the SBC LNA monomer LNA ^{2S}U.

The complement of a nucleic acid sequence as used herein refers to an oligonucleotide which, when aligned with the nucleic acid sequence such that the 5′ end of one sequence is paired with the 3′ end of the other, is in “antiparallel association.” Bases not commonly found in natural nucleic acids that may be included in the nucleic acids of the present invention include, for example, inosine and 7-deazaguanine. Complementarity may not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

Stability of a nucleic acid duplex is measured by the melting temperature, or “Tₘ”. The Tₘ of a particular nucleic acid duplex under specified conditions is the temperature at which half of the duplexes have disassociated.

The term “nucleobase” covers the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, Nucleic Acid Research, 25: 4429-4443, 1997. The term “nucleobase” thus includes not only the known purine and pyrimidine heterocycles, but also heterocyclic analogues and tautomers thereof. Further naturally and non naturally occurring nucleobases include those disclosed in U.S. Pat. No. 3,687,808; in chapter 15 by Sanghvi, in Antisense Research and Application, Ed. S. T. Crooke and B. Lebleu, CRC Press, 1993; in Englisch, et al., Angewandte Chemie, International Edition, 30: 613-722, 1991 (see, especially pages 622 and 623, and in the Concise Encyclopedia of Polymer Science and Engineering, J. I. Kroschwitz Ed., John Wiley & Sons, pages 858-859, 1990, Cook, Anti-Cancer DrugDesign 6: 585-607, 1991, each of which are hereby incorporated by reference in their entirety).

The term “nucleosidic base” or “nucleobase analogue” is further intended to include heterocyclic compounds that can serve as like nucleosidic bases including certain “universal bases” that are not nucleosidic bases in the most classical sense but serve as nucleosidic bases. Especially mentioned as a universal base is 3-nitropyrrole or a 5-nitroindole. Other preferred compounds include pyrene and pyridyloxazole derivatives, pyrenyl, pyrenylmethylglycerol derivatives and the like. Other preferred universal bases include, pyrrole, diazole or triazole derivatives, including those universal bases known in the art.

By “oligonucleotide,”“oligomer,” or “oligo” is meant a successive chain of monomers (e.g., glycosides of heterocyclic bases) connected via internucleoside linkages. The linkage between two successive monomers in the oligo consist of 2 to 4, desirably 3, groups/atoms selected from —CH₂—, —O—, —S—, —NR^{H}—, >C═O, >C═NR^{H}, >C═S, —Si(R″)₂—, —S—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆alkyl and phenyl. Illustrative examples of such linkages are —CH₂—CH₂—CH₂—, —CH₂—CO—CH₂—, —CH₂—CHOH—CH₂—, —O—CH₂—O—, —OoCH₂—CH₂—, —(O)—CH₂—CH═(including R⁵ when used as a linkage to a succeeding monomer), —CH₂—CH₂—(O)—, —NR^{H}—CH₂—CH₂—, —CH₂—CH₂—NR^{H}, —CH₂—NR^{H}—CH₂—, —(O)—CH₂—CH₂—NR^{H}—, —NR^{H}—C(O)—(O)—, —NR^{H}—CO—NR^{H}—, —NR^{H}—CS—NR^{H}—, —NR^{H}—C(═NR^{H})—NR^{H}—,—NR^{H}—CO—CH₂—NR^{H}—, —(O)—CO—O—, —O—CO—CH₂—O—, —(O)—CH₂—CO—O—, —CH₂—CO—NR^{H}—, —(O)—CO—NR^{H}, —NR^{H}—CO—CH₂—, —(O)—CH₂—CO—NR^{H}, —(O)—CH₂—CH₂—NR^{H}—, —CH═N—O—, ,CH₂—NR^{H}—O—, —CH₂—(O)—N═(including R⁵ when used as a linkage to a succeeding monomer), —CH₂—O—NR^{H}, —CO—NR^{H}—CH₂—, —CH₂—NR^{H}—(O)—, —CH₂—NR^{H}—CO—, —(O)—NR^{H}—CH₂—, —(O)—NR^{H}—, —O—CH₂—S—, —S—CH₂—O—, —CH₂—CH₂—S—, —O—CH₂—CH₂—S—, —S—CH₂—CH═(including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—N RH^{H}—, —NR^{H}—S(O)₂—CH₂—, —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}—, —NR^{H—P(O)}₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—, —O—P(O,S)—O—, —O—P(S)₂—O—, —NR^{H}—P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆alkyl and phenyl, are especially desirable. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443. The left-hand side of the internucleoside linkage is bound to the 5-membered ring as substituent P at the 3′-position, whereas the right-hand side is bound to the 5′-position of a preceding monomer.

By “LNA” or “LNA monomer” (e.g., an LNA nucleoside or LNA nucleotide) or an LNA oligomer (e.g., an oligonucleotide or nucleic acid) is meant a nucleoside or nucleotide analogue that includes at least one LNA monomer. LNA monomers as disclosed in PCT Publication WO 99/14226 are in general particularly desirable modified nucleic acids for incorporation into an oligonucleotide of the invention. Additionally, the nucleic acids may be modified at either the 3′ and/or 5′ end by any type of modification known in the art. For example, either or both ends may be capped with a protecting group, attached to a flexible linking group, attached to a reactive group to aid in attachment to the substrate surface, etc. Desirable LNA monomers and their method of synthesis also are disclosed in U.S. Pat. No. 6,043,060, U.S. Pat. No. 6,268,490, PCT Publications WO 01/07455, WO 01/00641, WO 98/39352, WO 00/56746, WO 00/56748 and WO 00/66604 as well as in the following papers: Morita et al., Bioorg. Med. Chem. Lett. 12(1):73-76, 2002; Hakansson et al., Bioorg. Med. Chem. Lett. 11(7):935-938, 2001; Koshkin et aL, J. Org. Chem. 66(25):8504-8512, 2001; Kvaerno etal., J. Org. Chem. 66(16):5498-5503, 2001; Hakansson et al., J. Org. Chem. 65(17):5161-5166, 2000; Kvaerno et al., J. Org. Chem. 65(17):5167-5176, 2000; Pfundheller et al., Nucleosides Nucleotides 18(9):2017-2030, 1999; and Kumar et al., Bioorg. Med. Chem. Lett. 8(16):2219-2222, 1998.

Preferred LNA monomers, also referred to as “oxy-LNA” are LNA monomers which include bicyclic compounds as disclosed in PCT Publication WO 03/020739 wherein the bridge between R^{4′} and R^{2′} as shown in formula (I) below together designate —CH₂—O— or —CH₂—CH₂—O—.

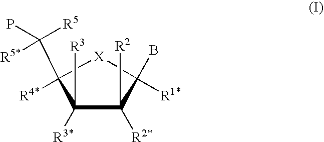

wherein X is selected from —O—, —S—, —N(R^{N})—, —C(R⁶R^{6*})—, —O—C(R⁷R^{7*}) —, —C(R⁶R^{6*})—O—, —S—C(R⁷R^{7*})—, —C(R⁶R^{6*})—S—, —N(R^{N*})—C(R⁷ R^{7*})—, —C(R⁶R^{6*})—N(R^{N*})—, and —C(R⁶R^{6*})—C(R⁷R^{7*}).

B is selected from a modified base as discussed above e.g. an optionally substituted carbocyclic aryl such as optionally substituted pyrene or optionally substituted pyrenylmethylglycerol, or an optionally substituted heteroalicylic or optionally substituted heteroaromatic such as optionally substituted pyridyloxazole, optionally substituted pyrrole, optionally substituted diazole or optionally substituted triazole moieties; hydrogen, hydroxy, optionally substituted C₁₋₄-alkoxy, optionally substituted C₁₋₄-alkyl, optionally substituted C₁₋₄-acyloxy, nucleobases, DNA intercalators, photo-chemically active groups, thermochemically active groups, chelating groups, reporter groups, and ligands.

P designates the radical position for an internucleoside linkage to a succeeding monomer, or a 5′-terminal group, such internucleoside linkage or 5′-terminal group optionally including the substituent R⁵. One of the substituents R², R², R³, and R^{3*} is a group P* which designates an internucleoside linkage to a preceding monomer, or a 2′/3′-terminal group. The substituents of R^{1*}, R^{4*}, R⁵, R^{5*}, R⁶, R^{6*}, R⁷, R^{7*}, R^{N}, and the ones of R², R^{2*}, R³, and R^{3*} not designating P* each designates a biradical comprising about 1-8 groups/atoms selected from —C(R^{a}R^{b})—, —C(R^{a})═C(R^{a})—, —C(R^{a})═N—, —C(R^{a})—O—, —O—, —Si(R^{a})₂—, —C(R^{a})—S, —S—, —SO₂—, —C(R^{a})—N(R^{b})—, —N(R^{a})—, and >C═Q, wherein Q is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, hetero-aryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆- alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents R^{a} and R^{b} together may designate optionally substituted methylene (═CH₂), and wherein two non-geminal or geminal substituents selected from R^{a}, R^{b}, and any of the substituents R^{1*}, R², R^{2*}, R³, R^{3*}, R^{4*}, R⁵, R^{5*}, R⁶ and R^{6*}, R⁷, and R^{7*} which are present and not involved in P, P^{*} or the biradical(s) together may form an associated biradical selected from biradicals of the same kind as defined before; the pair(s) of non-geminal substituents thereby forming a mono- or bicyclic entity together with (i) the atoms to which said non-geminal substituents are bound and (ii) any intervening atoms.

Each of the substituents R^{1*}, R², R^{2*}, R³, R^{4*}, R⁵, R^{5*}, R⁶ and R^{6*}, R⁷, and R^{7*} which are present and not involved in P, P^{*} or the biradical(s), is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di-(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N*}, when present and not involved in a biradical, is selected from hydrogen and C₁₋₄-alkyl; and basic salts and acid addition salts thereof.

Exemplary 5′, 3′, and/or 2′ terminal groups include —H, —OH, halo (e.g., chloro, fluoro, iodo, or bromo), optionally substituted aryl, (e.g., phenyl or benzyl), alkyl (e.g., methyl or ethyl), alkoxy (e.g., methoxy), acyl (e.g. acetyl or benzoyl), aroyl, aralkyl, hydroxy, hydroxyalkyl, alkoxy, aryloxy, aralkoxy, nitro, cyano, carboxy, alkoxycarbonyl, aryloxycarbonyl, aralkoxycarbonyl, acylamino, aroylamino, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, amidino, amino, carbamoyl, sulfamoyl, alkene, alkyne, protecting groups (e.g., silyl, 4,4′-dimethoxytrityl, monomethoxytrityl, or trityl(triphenylmethyl)), linkers (e.g., a linker containing an amine, ethylene glycol, quinone such as anthraquinone), detectable labels (e.g., radiolabels or fluorescent labels), and biotin.

It is understood that references herein to a nucleic acid unit, nucleic acid residue, LNA monomer, or similar term are inclusive of both individual nucleoside units and nucleotide units and nucleoside units and nucleotide units within an oligonucleotide.

A “modified base” or other similar terms refer to a composition (e.g., a non-naturally occurring nucleobase or nucleosidic base), which can pair with a natural base (e.g., adenine, guanine, cytosine, uracil, and/or thymine) and/or can pair with a non-naturally occurring nucleobase or nucleosidic base. Desirably, the modified base provides a Tₘ differential of 15, 12, 10, 8, 6, 4, or 2° C. or less as described herein. Exemplary modified bases are described in EP 1 072 679 and WO 97/12896.

The term “chemical moiety” refers to a part of a molecule. “Modified by a chemical moiety” thus refer to a modification of the standard molecular structure by inclusion of an unusual chemical structure. The attachment of said structure can be covalent or non-covalent.

The term “inclusion of a chemical moiety” in an oligonucleotide probe thus refers to attachment of a molecular structure. Such as chemical moiety include but are not limited to covalently and/or non-covalently bound minor groove binders (MGB) and/or intercalating nucleic acids (INA) selected from a group consisting of asymmetric cyanine dyes, DAPI, SYBR Green I, SYBR Green II, SYBR Gold, Pi-coGreen, thiazole orange, Hoechst 33342, Ethidium Bromide, 1-O-(1-pyrenylmethyl)glycerol and Hoechst 33258. Other chemical moieties include the modified nucleobases, nucleosidic bases or LNA modified oligonucleotides.

“Oligonucleotide analogue” refers to a nucleic acid binding molecule capable of recognizing a particular target nucleotide sequence. A particular oligonucleotide analogue is peptide nucleic acid (PNA) in which the sugar phosphate backbone of an oligonucleotide is replaced by a protein like backbone. In PNA, nucleobases are attached to the uncharged polyamide backbone yielding a chimeric pseudopeptide-nucleic acid structure, which is homomorphous to nucleic acid forms.

“High affinity nucleotide analogue” refers to a non-naturally occurring nucleotide analogue that increases the “binding affinity” of an oligonucleotide probe to its complementary recognition sequence when substituted with at least one such high-affinity nucleotide analogue.

As used herein, a probe with an increased “binding affinity” for a recognition sequence compared to a probe which comprises the same sequence but does not comprise a stabilizing nucleotide, refers to a probe for which the association constant (Kₐ) of the probe recognition segment is higher than the association constant of the complementary strands of a double-stranded molecule. In another preferred embodiment, the association constant of the probe recognition segment is higher than the dissociation constant (K_{d}) of the complementary strand of the recognition sequence in the target sequence in a double stranded molecule.

Monomers are referred to as being “complementary” if they contain nucleobases that can form hydrogen bonds according to Watson-Crick base-pairing rules (e.g. G with C, A with T or A with U) or other hydrogen bonding motifs such as for example diaminopurine with T, 5-methyl C with G, 2-thiothymidine with A, inosine with C, pseudoisocytosine with G, etc.

The term “succeeding monomer” relates to the neighbouring monomer in the 5′-terminal direction and the “preceding monomer” relates to the neighbouring monomer in the 3′-terminal direction.

The term “target nucleic acid” or “target ribonucleic acid” refers to any relevant nucleic acid of a single specific sequence, e.g., a biological nucleic acid, e.g., derived from a patient, an animal (a human or non-human animal), a plant, a bacteria, a fungi, an archae, a cell, a tissue, an organism, etc. For example, where the target ribonucleic acid or nucleic acid is derived from a bacteria, archae, plant, non-human animal, cell, fungi, or non-human organism, the method optionally further comprises selecting the bacteria, archae, plant, non-human animal, cell, fungi, or non-human organism based upon detection of the target nucleic acid. In one embodiment, the target nucleic acid is derived from a patient, e.g., a human patient. In this embodiment, the invention optionally further includes selecting a treatment, diagnosing a disease, or diagnosing a genetic predisposition to a disease, based upon detection of the target nucleic acid.

“Target sequence” refers to a specific nucleic acid sequence within any target nucleic acid.

The term “stringent conditions”, as used herein, is the “stringency” which occurs within a range from about Tₘ−5° C. (5° C. below the melting temperature (Tₘ) of the probe) to about 20° C. to 25° C. below Tₘ. As will be understood by those skilled in the art, the stringency of hybridization may be altered in order to identify or detect identical or related polynucleotide sequences. Hybridization techniques are generally described in Nucleic Acid Hybridization, A Practical Approach, Ed. Hames, B. D. and Higgins, S. J., IRL Press, 1985; Gall and Pardue, Proc. Natl. Acad. Sci., USA 63: 378-383, 1969; and John, et al. Nature 223: 582-587, 1969, each of which is hereby incorporated by reference. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least bout 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “modified backbone” is meant a nucleotide backbone structure other than the naturally occurring ribose-phosphate or deoxyribose-phosphate backbones. Exemplary modified backbones include a ribonucleotide moiety that is substituted at the 2′ position. The substituents at the 2′ position may, for example, be a saturated, unsaturated, unbranched, or branched C1 to C4 alkyl group, e.g., 2′-O-methyl ribose. Another suitable example of a substituent at the 2′ position of a modified ribonucleotide moiety is a C1 to C4 alkoxy-C1 to C4 alkyl group, e.g., methoxyethyl. Another suitable example of a modified ribonucleotide moiety is a ribonucleotide that is substituted at the 2′ position with fluoro group. Preferred modified backbones also include LNA.

By two nucleotides “disposed X nucleotides apart” is meant positioned in a nucleotide sequence so that X-1 nucleotides are disposed between the two nucleotides. For example, in the sequence ACTG, the A and G are disposed three nucleotides apart.

### DETAILED DESCRIPTION OF THE INVENTION

The present invention provides novel oligonucleotide compositions and probe sequences for the use in detection, isolation, purification, amplification, identification, quantification, or capture of miRNAs, their target mRNAs, stem-loop precursor miRNAs, siRNAs, other non-coding RNAs, RNA-edited transcripts or alternative mRNA splice variants characterized in that the probe sequences contain a number of nucleoside analogues.

In a preferred embodiment the number of nucleoside analogue corresponds to from 20 to 40% of the oligonucleotide of the invention.

In a preferred embodiment the probe sequences are substituted with a nucleoside analogue with regular spacing between the substitutions

In another preferred embodiment the probe sequences are substituted with a nucleoside analogue with irregular spacing between the substitutions

In a preferred embodiment the nucleoside analogue is LNA.

In a further preferred embodiment the detection probe sequences comprise a photochemically active group, a thermochemically active group, a chelating group, a reporter group, or a ligand that facilitates the direct of indirect detection of the probe or the immobilisation of the oligonucleotide probe onto a solid support.

In a further preferred embodiment

(a) the photochemically active group, the thermochemically active group, the chelating group, the reporter group, or the ligand includes a spacer (K), said spacer comprising a chemically cleavable group; or

(b) the photochemically active group, the thermochemically active group, the chelating group, the reporter group, or the ligand is attached via the biradical of at least one of the LNA(s) of the oligonucleotide.

Preferred uses include:

(a) capture and detection of naturally occurring or synthetic single stranded nucleic acids such as miRNAs, their target mRNAs, stem-loop precursor miRNAs, siRNAs, other non-coding RNAs, RNA-edited transcripts or alternative mRNA splice variants; or

(b) purification of naturally occurring single stranded nucleic acids such as miRNAs, their target mRNAs, stem-loop precursor miRNAs, siRNAs, other non-coding RNAs, RNA-edited transcripts or alternative mRNA splice variants; or

(c) detection and assessment of expression patterns naturally occurring single stranded nucleic acids such as miRNAs, their target mRNAs, stem-loop precursor miRNAs, siRNAs, other non-coding RNAs, RNA-edited transcripts or alternative mRNA splice variants by RNA in-situ hybridisation, dot blot hybridisation, reverse Ddot blot hybridisation, or in Northem blot analysis or expression profiling by microarrays

(d) functional analysis of naturally occurring single stranded nucleic acids such as miRNAs, their target mRNAs, stem-loop precursor miRNAs, siRNAs, other non-coding RNAs, RNA-edited transcripts or altemative mRNA splice variants in vitro and in vivo in plants or animals, such as human, mouse, rat, zebrafish, Caenorhabditis elegans, Drosophila melanogaster, Arabidopsis thaliana, rice and maize, by inhibiting their mode of action, e.g. the binding of mature miRNAs to their cognate target mRNAs.

Further embodiments includes the use of an LNA modified oligonucleotide probe as an aptamer in molecular diagnostics or (b) as an aptamer in RNA mediated catalytic processes or (c) as an aptamer in specific binding of antibiotics, drugs, amino acids, peptides, structural proteins, protein receptors, protein enzymes, saccharides, polysaccharides, biological cofactors, nucleic acids, or triphosphates or (d) as an aptamer in the separation of enantiomers from racemic mixtures by stereo-specific binding or (e) for labelling cells or (f) to hybridise to non-protein coding cellular RNAs, such as tRNA, rRNA, snRNA and scRNA, in vivo or in-vitro or (g) to hybridise to non-protein coding cellular RNAs, such as tRNA, rRNA, snRNA and scRNA, in vivo or in-vitro or (h) in the construction of Taqman probes or Molecular Beacons.

The present invention also provides a kit for the isolation, purification, amplification, detection, identification, quantification, or capture of natural or synthetic nucleic acids, where the kit comprises a reaction body and one or more LNAs as defined herein. The LNAs are preferably immobilised onto said reactions body (e.g. by using the immobilising techniques described above).

For the kits according to the invention, the reaction body is preferably a solid support material, e.g. selected from borosilicate glass, soda-lime glass, polystyrene, polycarbonate, polypropylene, polyethylene, polyethyleneglycol terephthalate, polyvinylacetate, polyvinylpyrrolidinone, polymethylmethacrylate and polyvinylchloride, preferably polystyrene and polycarbonate. The reaction body may be in the form of a specimen tube, a vial, a slide, a sheet, a film, a bead, a pellet, a disc, a plate, a ring, a rod, a net, a filter, a tray, a microtitre plate, a stick, or a multi-bladed stick.

A written instruction sheet stating the optimal conditions for use of the kit typically accompanies the kits.

Further aspects of the invention

Once the appropriate target RNA sequences have been selected, LNA substituted detection probes are preferably chemically synthesized using commercially available methods and equipment as described in the art (Tetrahedron 54: 3607-30, 1998). For example, the solid phase phosphoramidite method can be used to produce short LNA probes (Caruthers, et al., Cold Spring Harbor Symp. Quant. Biol. 47:411-418, 1982, Adams, et al., J. Am. Chem. Soc. 105: 661 (1983).

LNA-containing-probes can be labelled during synthesis. The flexibility of the phosphoramidite synthesis approach furthermore facilitates the easy production of LNAs carrying all commercially available linkers, fluorophores and labelling-molecules available for this standard chemistry. LNA-modified probes may also be labelled by enzymatic reactions e.g. by kinasing using T4 polynucleotide kinase and gamma-³²P-ATP or by using terminal deoxynucleotidyl transferase (TDT) and any given digoxygenin-conjugated nucleotide triphosphate (dNTP) or dideoxynucleotide triphosphate (ddNTP).

Detection probes according to the invention can comprise single labels or a plurality of labels. In one aspect, the plurality of labels comprise a pair of labels which interact with each other either to produce a signal or to produce a change in a signal when hybridization of the detection probe to a target sequence occurs.

In another aspect, the detection probe comprises a fluorophore moiety and a quencher moiety, positioned in such a way that the hybridized state of the probe can be distinguished from the unhybridized state of the probe by an increase in the fluorescent signal from the nucleotide. In one aspect, the detection probe comprises, in addition to the recognition element, first and second complementary sequences, which specifically hybridize to each other, when the probe is not hybridized to a recognition sequence in a target molecule, bringing the quencher molecule in sufficient proximity to said reporter molecule to quench fluorescence of the reporter molecule. Hybridization of the target molecule distances the quencher from the reporter molecule and results in a signal, which is proportional to the amount of hybridization.

In the present context, the term “label” means a reporter group, which is detectable either by itself or as a part of a detection series. Examples of functional parts of reporter groups are biotin, digoxigenin, fluorescent groups (groups which are able to absorb electromagnetic radiation, e.g. light or X-rays, of a certain wavelength, and which subsequently reemits the energy absorbed as radiation of longer wavelength; illustrative examples are DANSYL (5-dimethylamino)-1-naphthalenesulfonyl), DOXYL (N-oxyl-4,4-dimethyloxazolidine), PROXYL (N-oxyl-2,2,5,5-tetramethylpyrrolidine), TEMPO (N-oxyl-2,2,6,6-tetramethylpiperidine), dinitrophenyl, acridines, coumarins, Cy3 and Cy5 (trademarks for Biological Detection Systems, Inc.), erythrosine, coumaric acid, umbelliferone, Texas red, rhodamine, tetramethyl rhodamine, Rox, 7-nitrobenzo-2-oxa-1-diazole (NBD), pyrene, fluorescein, Europium, Ruthenium, Samarium, and other rare earth metals), radio isotopic labels, chemiluminescence labels (labels that are detectable via the emission of light during a chemical reaction), spin labels (a free radical (e.g. substituted organic nitroxides) or other paramagnetic probes (e.g. Cu²⁺, Mg²⁺) bound to a biological molecule being detectable by the use of electron spin resonance spectroscopy). Especially interesting examples are biotin, fluorescein, Texas Red, rhodamine, dinitrophenyl, digoxigenin, Ruthenium, Europium, Cy5, Cy3, etc.

Suitable samples of target nucleic acid molecules may comprise a wide range of eukaryotic and prokaryotic cells, including protoplasts; or other biological materials, which may harbour target nucleic acids. The methods are thus applicable to tissue culture animal cells, animal cells (e.g., blood, serum, plasma, reticulocytes, lymphocytes, urine, bone marrow tissue, cerebrospinal fluid or any product prepared from blood or lymph) or any type of tissue biopsy (e.g. a muscle biopsy, a liver biopsy, a kidney biopsy, a bladder biopsy, a bone biopsy, a cartilage biopsy, a skin biopsy, a pancreas biopsy, a biopsy of the intestinal tract, a thymus biopsy, a mammae biopsy, a uterus biopsy, a testicular biopsy, an eye biopsy or a brain biopsy, e.g., homogenized in lysis buffer), archival tissue nucleic acids, plant cells or other cells sensitive to osmotic shock and cells of bacteria, yeasts, viruses, mycoplasmas, protozoa, rickettsia, fungi and other small microbial cells and the like.

Preferably, the detection probes of the invention are modified in order to increase the binding affinity of the probes for the target sequence by at least two-fold compared to probes of the same sequence without the modification, under the same conditions for hybridization or stringent hybridization conditions. The preferred modifications include, but are not limited to, inclusion of nucleobases, nucleosidic bases or nucleotides that have been modified by a chemical moiety or replaced by an analogue to increase the binding affinity. The preferred modifications may also include attachment of duplex-stabilizing agents e.g., such as minor-groove-binders (MGB) or intercalating nucleic acids (INA). Additionally, the preferred modifications may also include addition of non-discriminatory bases e.g., such as 5-nitroindole, which are capable of stabilizing duplex formation regardless of the nucleobase at the opposing position on the target strand. Finally, multi-probes composed of a non-sugar-phosphate backbone, e.g. such as PNA, that are capable of binding sequence specifically to a target sequence are also considered as a modification. All the different binding affinity-increasing modifications mentioned above will in the following be referred to as “the stabilizing modification(s)”, and the tagging probes and the detection probes will in the following also be referred to as “modified oligonucleotide”. More preferably the binding affinity of the modified oligonucleotide is at least about 3-fold, 4-fold, 5-fold, or 20-fold higher than the binding of a probe of the same sequence but without the stabilizing modification(s).

Most preferably, the stabilizing modification(s) is inclusion of one or more LNA nucleotide analogs. Probes from 6 to 30 nucleotides according to the invention may comprise from 1 to 8 stabilizing nucleotides, such as LNA nucleotides. When at least two LNA nucleotides are included, these may be consecutive or separated by one or more non-LNA nucleotides. In one aspect, LNA nucleotides are alpha and/or xylo LNA nucleotides.

The problems with existing detection, quantification and knock-down of miRNAs and siRNAs as outlined above are addressed by the use of the novel oligonucleotide probes of the invention in combination with any of the methods of the invention selected so as to recognize or detect a majority of all discovered and detected miRNAs, in a given cell type from a given organism. In one aspect, the probe sequences comprise probes that detect mammalian mature miRNAs, e.g., such as mouse, rat, rabbit, monkey, or human miRNAs. By providing a sensitive and specific method for detection of mature miRNAs, the present invention overcomes the limitations discussed above especially for conventional miRNA assays and siRNA assays. The detection element of the detection probes according to the invention may be single or double labelled (e.g. by comprising a label at each end of the probe, or an internal position). In one aspect, the detection probe comprises two labels capable of interacting with each other to produce a signal or to modify a signal, such that a signal or a change in a signal may be detected when the probe hybridizes to a target sequence. A particular aspect is when the two labels comprise a quencher and a reporter molecule.

In another aspect, the probe comprises a target-specific recognition segment capable of specifically hybridizing to a target molecule comprising the complementary recognition sequence. A particular detection aspect of the invention referred to as a “molecular beacon with a stem region” is when the recognition segment is flanked by first and second complementary hairpin-forming sequences which may anneal to form a hairpin. A reporter label is attached to the end of one complementary sequence and a quenching moiety is attached to the end of the other complementary sequence. The stem formed when the first and second complementary sequences are hybridized (i.e., when the probe recognition segment is not hybridized to its target) keeps these two labels in close proximity to each other, causing a signal produced by the reporter to be quenched by fluorescence resonance energy transfer (FRET). The proximity of the two labels is reduced when the probe is hybridized to a target sequence and the change in proximity produces a change in the interaction between the labels. Hybridization of the probe thus results in a signal (e.g. fluorescence) being produced by the reporter molecule, which can be detected and/or quantified.

The invention also provides a method, system and computer program embedded in a computer readable medium (“a computer program product”) for designing detection probes comprising at least one stabilizing nucleobase. The method comprises querying a database of target sequences (e.g., such as the miRNA registry at http://www.sancier.ac.uk/Software/Rfam/mima/index.shtml) and designing probes which: i) have sufficient binding stability to bind their respective target sequence under stringent hybridization conditions, ii) have limited propensity to form duplex structures with itself, and iii) are capable of binding to and detecting/quantifying at least about 60%, at least about 70%, at least about 80%, at least about 90% or at least about 95% of all the target sequences in the given database of miRNAs or other RNA sequences.

In one preferred aspect, the target sequence database comprises nucleic acid sequences corresponding to human, mouse, rat, Drosophila melanogaster, C. elegans, Arabidopsis thaliana, maize or rice miRNAs.

In another aspect, the method further comprises calculating stability based on the assumption that the recognition sequence comprises at least one stabilizing nucleotide, such as an LNA molecule. In one preferred aspect the calculated stability is used to eliminate probes with inadequate stability from the database of virtual candidate probes prior to the initial query against the database of target sequence to initiate the identification of optimal probe recognition sequences.

In another aspect, the method further comprises calculating the capability for a given probe sequence to form a duplex structure with itself based on the assumption that the sequence comprises at least one stabilizing nucleotide, such as an LNA molecule. In one preferred aspect the calculated propensity is used to eliminate probe sequences that are likely to form probe duplexes from the database of virtual candidate probes.

A preferred embodiment of the invention are kits for the detection or quantification of target miRNAs, siRNAs, RNA-edited transcripts, non-coding antisense transcripts or alternative splice variants comprising libraries of detection probes. In one aspect, the kit comprises in silico protocols for their use. The detection probes contained within these kits may have any or all of the characteristics described above. In one preferred aspect, a plurality of probes comprises at least one stabilizing nucleotide, such as an LNA nucleotide. In another aspect, the plurality of probes comprises a nucleotide coupled to or stably associated with at least one chemical moiety for increasing the stability of binding of the probe. The kits according to the invention allow a user to quickly and efficiently develop an assay for different miRNA targets, siRNA targets, RNA-edited transcripts, non-coding antisense transcripts or alternative splice variants.

In general, the invention features the design of high affinity oligonucleotide probes that have duplex stabilizing properties and methods highly useful for a variety of target nucleic acid detection methods (e.g., monitoring spatiotemporal expression of microRNAs or siRNAs or knock-down of miRNAs). Some of these oligonucleotide probes contain novel nucleotides created by combining specialized synthetic nucleobases with an LNA backbone, thus creating high affinity oligonucleotides with specialized properties such as reduced sequence discrimination for the complementary strand or reduced ability to form intramolecular double stranded structures. The invention also provides improved methods for detecting and quantifying ribonucleic acids in complex nucleic acid sample. Other desirable modified bases have decreased ability to self-anneal or to form duplexes with oligonucleotide probes containing one or more modified bases.

### EXAMPLES

The invention will now be further illustrated with reference to the following examples. It will be appreciated that what follows is by way of example only and that modifications to detail may be made while still falling within the scope of the invention.

### Example 1

### Synthesis, Deprotection and Purification of LNA-substituted Oli-gonucleotide Probes

The LNA-substituted probes of Example 2 to 11 were prepared on an automated DNA synthesizer (Expedite 8909 DNA synthesizer, PerSeptive Biosystems, 0.2 μmol scale) using the phosphoramidite approach (Beaucage and Caruthers, Tetrahedron Lett. 22: 1859-1862, 1981) with 2-cyanoethyl protected LNA and DNA phosphoramidites, (Sinha, et al., Tetrahedron Lett. 24: 5843-5846, 1983). CPG solid supports derivatised with a suitable quencher and 5′-fluorescein phosphoramidite (GLEN Research, Sterling, Va., USA). The synthesis cycle was modified for LNA phosphoramidites (250s coupling time) compared to DNA phosphoramidites. 1H-tetrazole or 4,5-dicyanoimidazole (Proligo, Hamburg, Germany) was used as activator in the coupling step.

The probes were deprotected using 32% aqueous ammonia (1 h at room temperature, then 2 hours at 60° C.) and purified by HPLC (Shimadzu-SpectraChrom series; Xterra™ RP18 column, 10?m 7.8×150 mm (Waters). Buffers: A: 0.05M Triethylammonium acetate pH 7.4. B. 50% acetonitrile in water. Eluent: 0-25 min: 10-80% B; 25-30 min: 80% B). The composition and purity of the probes were verified by MALDI-MS (PerSeptive Biosystem, Voyager DE-PRO) analysis.

### Example 2

### List of LNA-substituted Detection Probes for Detection of Fully Conserved Vertebrate microRNAs in all Vertebrates

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH2-C6- or a NH2-C6-hexaethylene glycol monomer or dimer group at the 5′-end or at the 3′-end of the probes during synthesis.

| | | Self- | Calcu- |
|---|---|---|---|
| | | comp | lated |
| LNA probe name | Sequence 5′-3′ | score | Tm |
| hsa-let7f/LNA | aamCtaTacAatmCtamCt | 16 | 67 |
| | amCctmCa | | |
| hsa-miR19b/LNA | tmCagTttTgcAtgGatTt | 34 | 75 |
| | gmCaca | | |
| hsa-miR17-5p/LNA | actAccTgcActGtaAgcA | 39 | 74 |
| | ctTtg | | |
| hsa-miR217/LNA | atcmCaaTcaGttmCctGa | 49 | 75 |
| | tGcaGta | | |
| hsa-miR218/LNA | acAtgGttAgaTcaAgcAc | 40 | 70 |
| | aa | | |
| hsa-miR222/LNA | gaGacmCcaGtaGccAgaT | 38 | 80 |
| | gtAgct | | |
| hsa-let7i/LNA | agmCacAaamCtamCtamC | 18 | 71 |
| | ctmCa | | |
| hsa-miR27b/LNA | cagAacTtaGccActGtg | 35 | 68 |
| | Aa | | |
| hsa-miR301/LNA | gctTtgAcaAtamCtaTtg | 36 | 70 |
| | mCacTg | | |
| hsa-miR30b/LNA | gcTgaGtgTagGatGttTa | 33 | 70 |
| | ca | | |
| hsa-miR100/LNA | cacAagTtcGgaTctAcgG | 38 | 77 |
| | gtt | | |
| hsa-miR34a/LNA | aamCaamCcaGctAagAca | 27 | 80 |
| | mCtgmCca | | |
| hsa-miR7/LNA | aacAaaAtcActAgtmCtt | 30 | 66 |
| | mCca | | |
| hsa-miR125b/LNA | tcamCaaGttAggGtcTca | 35 | 77 |
| | Ggga | | |
| hsa-miR133a/LNA | acAgcTggTtgAagGggAc | 41 | 82 |
| | cAa | | |
| hsa-miR101/LNA | cttmCagTtaTcamCagTa | 54 | 68 |
| | cTgta | | |
| hsa-miR108/LNA | aatGccmCctAaaAatmCc | 23 | 66 |
| | tTat | | |
| hsa-miR107/LNA | tGatAgcmCctGtamCaaT | 63 | 80 |
| | gcTgct | | |
| hsa-miR153/LNA | tcamCttTtgTgamCtaTg | 35 | 68 |
| | cAa | | |
| hsa-miR10b/LNA | amCaaAttmCggTtcTacA | 35 | 73 |
| | ggGta | | |
| mmu-miR10b/LNA | acamCaaAttmCggTtcTa | 27 | 73 |
| | cAggg | | |
| hsa-miR194/LNA | tccAcaTggAgtTgcTgtT | 41 | 75 |
| | aca | | |
| hsa-miR199a/LNA | gaAcaGgtAgtmCtgAacA | 40 | 78 |
| | ctGgg | | |
| hsa-miR199a*/LNA | aacmCaaTgtGcaGacTac | 39 | 74 |
| | Tgta | | |
| hsa-miR20/LNA | ctAccTgcActAtaAgcAc | 26 | 70 |
| | tTta | | |
| hsa-miR214/LNA | ctGccTgtmCtgTgcmCtg | 30 | 81 |
| | mCtgt | | |
| hsa-miR219/LNA | agAatTgcGttTggAcaAt | 35 | 70 |
| | ca | | |
| hsa-miR223/LNA | gGggTatTtgAcaAacTga | 40 | 73 |
| | mCa | | |
| hsa-miR23a/LNA | gGaaAtcmCctGgcAatGt | 37 | 76 |
| | gAt | | |
| hsa-miR24/LNA | cTgtTccTgcTgaActGag | 35 | 80 |
| | mCca | | |
| hsa-miR26a/LNA | agcmCtaTccTggAttAct | 34 | 70 |
| | Tgaa | | |
| hsa-miR126/LNA | gcAttAttActmCacGgtA | 25 | 71 |
| | cga | | |
| hsa-miR126*/LNA | cgmCgtAccAaaAgtAatA | 28 | 68 |
| | atg | | |
| hsa-miR128a/LNA | aaAagAgamCcgGttmCac | 47 | 77 |
| | TgtGa | | |
| mmu-miR7b/LNA | aamCaaAatmCacAagTct | 24 | 68 |
| | Tcca | | |
| hsa-let7c/LNA | aamCcaTacAacmCtamCt | 11 | 74 |
| | amCctmCa | | |
| hsa-let7b/LNA | aamCcamCacAacmCtamC | 6 | 77 |
| | tamCctmCa | | |
| hsa-miR103/LNA | tmCatAgcmCctGtamCaa | 63 | 80 |
| | TgcTgct | | |
| hsa-miR129/LNA | agcAagmCccAgamCcgmC | 21 | 80 |
| | aaAaag | | |
| rno-miR129*/LNA | aTgcTttTtgGggTaaGgg | 37 | 78 |
| | mCtt | | |
| hsa-miR130a/LNA | gcmCctTttAacAttGcam | 34 | 70 |
| | Ctg | | |
| hsa-miR132/LNA | cgAccAtgGctGtaGacTg | 48 | 76 |
| | tTa | | |
| hsa-miR135a/LNA | tcamCatAggAatAaaAag | 22 | 69 |
| | mCcaTa | | |
| hsa-miR137/LNA | cTacGcgTatTctTaaGca | 48 | 68 |
| | Ata | | |
| hsa-miR200a/LNA | acaTcgTtamCcaGacAgt | 39 | 72 |
| | Gtta | | |
| hsa-miR142-3p/LNA | tmCcaTaaAgtAggAaamC | 29 | 72 |
| | acTaca | | |
| hsa-miR142-5p/LNA | gtaGtgmCttTctActTta | 36 | 63 |
| | Tg | | |
| hsa-miR181b/LNA | aamCccAccGacAgcAatG | 30 | 81 |
| | aaTgtt | | |
| hsa-miR183/LNA | caGtgAatTctAccAgtGc | 32 | 73 |
| | cAta | | |
| hsa-miR190/LNA | acmCtaAtaTatmCaaAca | 31 | 62 |
| | TatmCa | | |
| hsa-miR193/LNA | ctGggActTtgTagGccAg | 31 | 76 |
| | tt | | |
| hsa-miR19a/LNA | tmCagTttTgcAtaGatTt | 37 | 72 |
| | gmCaca | | |
| hsa-miR204/LNA | cagGcaTagGatGacAaaG | 25 | 78 |
| | ggAa | | |
| hsa-miR205/LNA | caGacTccGgtGgaAtgAa | 39 | 81 |
| | gGa | | |
| hsa-miR216/LNA | camCagTtgmCcaGctGag | 64 | 74 |
| | Atta | | |
| hsa-miR221/LNA | gAaamCccAgcAgamCaaT | 31 | 80 |
| | gtAgct | | |
| hsa-miR25/LNA | tcaGacmCgaGacAagTgc | 27 | 77 |
| | Aatg | | |
| hsa-miR29c/LNA | taamCcgAttTcaAatGgt | 47 | 70 |
| | Gcta | | |
| hsa-miR29b/LNA | amCacTgaTttmCaaAtgG | 47 | 71 |
| | tgmCta | | |
| hsa-miR30c/LNA | gmCtgAgaGtgTagGatGt | 33 | 73 |
| | tTaca | | |
| hsa-miR140/LNA | ctAccAtaGggTaaAacmC | 43 | 71 |
| | act | | |
| hsa-miR9*/LNA | acTttmCggTtaTctAgc | 27 | 65 |
| | Ttta | | |
| hsa-miR92/LNA | amCagGccGggAcaAgtGc | 36 | 81 |
| | aAta | | |
| hsa-miR96/LNA | aGcaAaaAtgTgcTagTgc | 38 | 75 |
| | mCaaa | | |
| hsa-miR99a/LNA | cacAagAtcGgaTctAcgG | 42 | 77 |
| | gtt | | |
| hsa-miR145/LNA | aAggGatTccTggGaaAac | 50 | 79 |
| | TggAc | | |
| hsa-miR155/LNA | ccmCctAtcAcgAttAgcA | 29 | 71 |
| | ttAa | | |
| hsa-miR29a/LNA | aamCcgAttTcaAatGgtG | 47 | 75 |
| | ctAg | | |
| mo-miR140*/LNA | gtcmCgtGgtTctAccmCt | 49 | 81 |
| | gTggTa | | |
| hsa-miR206/LNA | ccamCacActTccTtamCa | 11 | 73 |
| | tTcca | | |
| hsa-miR124a/LNA | tggmCatTcamCcgmCgtG | 43 | 80 |
| | ccTtaa | | |
| hsa-miR122a/LNA | acAaamCacmCatTgtmCa | 25 | 78 |
| | cActmCca | | |
| hsa-miR1/LNA | tamCatActTctTtamCat | 11 | 64 |
| | Tcca | | |
| hsa-miR181a/LNA | acTcamCcgAcaGcgTtgA | 49 | 77 |
| | atGtt | | |
| hsa-miR10a/LNA | cAcaAatTcgGatmCtamC | 37 | 74 |
| | agGgta | | |
| hsa-miR196a/LNA | ccaAcaAcaTgaAacTacm | 20 | 67 |
| | Cta | | |
| hsa-let7a/LNA | aamCtaTacAacmCtamCt | 16 | 70 |
| | amCctmCa | | |
| hsa-miR9/LNA | tcAtamCagmCtaGatAac | 34 | 71 |
| | mCaaAga | | |

### Example 3

### List of LNA-substituted Detection Probes for Detection of Fully Conserved Vertebrate microRNAs in all Vertebrates

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH2-C6- or a NH2-C6-hexaethylene glycol monomer or dimer group at the 5′-end or at the 3′-end of the probes during synthesis.

| | | Self- | Calcu- |
|---|---|---|---|
| | | compl | lated |
| Probe name | Sequence 5′-3′ | score | Tm |
| hsa-miR-210 | agcmCgcTgtmCacAcgmCacAg | 37 | 84 |
| hsa-miR-144 | taGtamCatmCatmCtaTacTgta | 37 | 64 |
| hsa-miR-338 | caAcaAaaTcamCtgAtgmCtgGa | 33 | 72 |
| hsa-miR-187 | ggcTgcAacAcaAgamCacGa | 30 | 79 |
| hsa-miR-200b | cAtcAttAccAggmCagTatTaga | 29 | 71 |
| hsa-miR-184 | cmCctTatmCagTtcTccGtcmCa | 23 | 75 |
| hsa-miR-27a | gcGgaActTagmCcamCtgTgaa | 35 | 77 |
| hsa-miR-215 | ctgTcaAttmCatAggTcat | 38 | 65 |
| hsa-miR-203 | agTggTccTaaAcaTttmCac | 23 | 68 |
| hsa-miR-16 | ccaAtaTttAcgTgcTgcTa | 30 | 68 |
| hsa-miR-152 | aAgtTctGtcAtgmCacTga | 29 | 72 |

### Example 4

### List of LNA-substituted Detection Probes for Detection of Zebrafish microRNAs

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluoro-chromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH2-C6- or a NH2-C6-hexaethylene glycol monomer or dimer group at the 5′-end or at the 3′-end of the probes during synthesis.

| | | Self- | Calcu- |
|---|---|---|---|
| | | comp | lated |
| Probe name | Sequence 5′-3′ | score | Tm |
| dre-miR-93 | ctAccTgcAcaAacAgcActTt | 26 | 73 |
| dre-miR-22 | acaGttmCttmCagmCtgGcaG | 62 | 76 |
| | ctt | | |
| dre-miR-213 | gGtamCagTcaAcgGtcGatG | 63 | 80 |
| | gt | | |
| dre-miR-31 | cagmCtaTgcmCaamCatmCtt | 34 | 76 |
| | Gcc | | |
| dre-miR-189 | amCtgTtaTcaGctmCagTagG | 41 | 75 |
| | cac | | |
| dre-miR-18 | tatmCtgmCacTaaAtgmCacm | 45 | 69 |
| | Ctta | | |
| dre-miR-15a | cAcaAacmCatTctGtgmCtgm | 35 | 74 |
| | Cta | | |
| dre-miR-34b | cAatmCagmCtaAcaAcamCtg | 24 | 74 |
| | mCcta | | |
| dre-miR-148a | acaAagTtcTgtAatGcamCt | 44 | 69 |
| | ga | | |
| dre-miR-125a | camCagGttAagGgtmCtcAgg | 38 | 80 |
| | Ga | | |
| dre-miR-139 | agAcamCatGcamCtgTaga | 34 | 69 |
| dre-miR-150 | cacTggTacAagGatTggGaga | 30 | 75 |
| dre-miR-192 | ggcTgtmCaaTtcAtaGgtmCa | 46 | 73 |
| dre-miR-98 | aacAacAcaActTacTacmCt | 17 | 68 |
| | ca | | |
| dre-let-7g | amCtgTacAaamCaamCtamCc | 30 | 73 |
| | tmCa | | |
| dre-miR-30a-5p | gctTccAgtmCggGgaTgtTta | 45 | 80 |
| | mCa | | |
| dre-miR-26b | aacmCtaTccTggAttActTg | 36 | 68 |
| | aa | | |
| dre-miR-21 | cAacAccAgtmCtgAtaAgcTa | 35 | 72 |
| dre-miR-146 | accmCttGgaAttmCagTtcT | 40 | 72 |
| | ca | | |
| dre-miR-182 | tgtGagTtcTacmCatTgcmCa | 32 | 72 |
| | aa | | |
| dre-miR-182* | taGttGgcAagTctAgaAcca | 32 | 72 |
| dre-miR-220 | aAgtGtcmCgaTacGgtTgtGg | 47 | 81 |

### Example 5

### List of LNA-substituted Detection Probes for Detection of Drosophila melanogaster microRNAs

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH2-C6- or a NH2-C6-hexaethylene glycol monomer or dimer group at the 5′-end or at the 3′-end of the probes during synthesis.

| | | Self- | Calcu- |
|---|---|---|---|
| | | compl | lated |
| Probe name | Sequence 5′-3′ | score | Tm |
| dme-miR-2c | gcmCcaTcaAagmCtgGctGtgAta | 68 | 78 |
| dme-miR-6 | aaaAagAacAgcmCacTgtGata | 36 | 71 |
| dme-miR-7 | amCaamCaaAatmCacTagTctTcca | 30 | 71 |
| dme-miR-14 | tAggAgaGagAaaAagActGa | 15 | 71 |
| dme-miR-277 | tgTcgTacmCagAtaGtgmCatTta | 38 | 72 |
| dme-miR-278 | aaAcgGacGaaAgtmCccAccGa | 41 | 80 |
| dme-miR-279 | tTaaTgaGtgTggAtcTagTca | 40 | 70 |
| dme-miR-309 | tAggAcaAacTttAccmCagTgc | 37 | 74 |
| dme-miR-310 | aAagGccGggAagTgtGcaAta | 28 | 79 |
| dme-miR-318 | tgaGatAaamCaaAgcmCcaGtga | 25 | 73 |
| dme-miR- | aaTcaGctTtcAaaAtgAtcTca | 40 | 66 |
| bantam | | | |

### Example 6

### List of LNA-substituted Detection Probes for Detection of Drosophila melanogaster and Caenorhabditis elegans microRNAs

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH2-C6- or a NH2-C6-hexaethylene glycol monomer or dimer group at the 5′-end or at the 3′-end of the probes during synthesis.

| | | Self- | Calcu- |
|---|---|---|---|
| | | comp | lated |
| Probe name | Sequence 5′-3′ | score | Tm |
| dme_cel- | cAtamCttmCttTacAttmCca | 14 | 62 |
| miR1/LNA | | | |
| dme_cel- | tcaAagmCtgGctGtgAta | 56 | 67 |
| miR2/LNA | | | |
| cel-lin4/LNA | tcAcamCttGagGtcTcag | 50 | 68 |

### Example 7

### List of LNA-substituted Detection Probes for Detection of Arabidopsis thaliana microRNAs

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH2-C6- or a NH2-C6-hexaethylene glycol monomer or dimer group at the 5′-end or at the 3′-end of the probes during synthesis.

| | | Self- | Calcu- |
|---|---|---|---|
| | | comp | lated |
| Probe name | Sequence 5′-3′ | score | Tm |
| ath-MIR171_LNA2 | gAtAtTgGcGcGgmCtmCaAt | 64 | 83 |
| | mCa | | |
| ath-MIR171_LNA3 | gAtaTtgGcgmCggmCtcAat | 54 | 78 |
| | mCa | | |
| ath-MIR159_LNA2 | tAgAgmCtmCcmCtTcAaTcm | 46 | 79 |
| | CaAa | | |
| ath-MIR159_LNA3 | tAgaGctmCccTtcAatmCca | 43 | 72 |
| | Aa | | |
| ath-MIR161LNA3 | cmCccGatGtaGtcActTtc | 34 | 73 |
| | Aa | | |
| ath-MIR167LNA3 | tAgaTcaTgcTggmCagmCtt | 53 | 79 |
| | mCa | | |
| ath-MIR319LNA3 | ggGagmCtcmCctTcaGtcmC | 70 | 78 |
| | aa | | |

### Example 8

### List of LNA-substituted Detection Probes for Detection of Arabidopsis thaliana microRNAs

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH2-C6- or a NH2-C6-hexaethylene glycol monomer or dimer group at the 5′-end or at the 3′-end of the probes during synthesis.

| | | Predicted |
|---|---|---|
| Oligo name | Sequence 5′-3′ | Tm ° C. |
| ath-miR159a/LNA | tAgaGctmCccTtcAatmCcaAa | 145 |
| ath-miR319a/LNA | ggGagmCtcmCctTcaGtcmCaa | 183 |
| ath-miR396a/LNA | gTtcAagAaaGctGtgGaa | 242 |
| ath-miR156a/LNA | gtgmCtcActmCtcTtcTgtmCa | 235 |
| ath-miR172a/LNA | atgmCagmCatmCatmCaaGatT | 228 |
| | ct | |

### Example 9

### List of LNA-substituted Detection Probes Useful as Negative Controls in Detection of Vertebrate microRNAs

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH2-C6- or a NH2-C6-hexaethylene glycol monomer or dimer group at the 5′-end or at the 3′-end of the probes during synthesis.

| | | Self- |
|---|---|---|
| | | comp |
| Probe name | Sequence 5′-3′ | score |
| hsa-miR206/LNA/2MM | ccamCacActmCtcTtamCatT | 8 |
| | cca | |
| hsa-miR206/LNA/MM10 | ccamCacActmCccTtamCatT | 8 |
| | cca | |
| hsa-miR124a/LNA/2MM | tggmCatTcaAagmCgtGccTt | 60 |
| | aa | |
| hsa-miR124a/LNA/MM10 | tggmCatTcaAcgmCgtGccTt | 60 |
| | aa | |
| hsa-miR122a/LNA/2MM | acAaamCacmCacmCgtmCacA | 18 |
| | ctmCca | |
| hsa-miR122a/LNA/MM11 | acAaamCacmCatmCgtmCacA | 18 |
| | ctmCca | |

### Example 10

### List of LNA-substituted Detection Probes for Detection of Human microRNAs

LNA nucleotides are depicted by capital letters, DNA nucleotides by lowercase letters, mC denotes LNA methyl-cytosine, PM perfect match to the miRNA, MM one mismatch at the central position of the probe sequence. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH2-C6- or a NH2-C6-hexaethylene glycol monomer or dimer group at the 5′-end or at the 3′-end of the probes during synthesis.

| | |
|---|---|
| Probe name | Sequence 5′-3′ |

| | |
|---|---|
| hsa-let7a/LNA_PM | aamCtaTacAacmCtamCtamCctmCa |
| hsa-let7f/LNA_PM | aamCtaTacAatmCtamCtamCctmCa |
| hsa-miR143LNA_PM | tGagmCtamCagTgcTtcAtcTca |
| hsa-miR145/LNA_PM | aAggGatTccTggGaaAacTggAc |
| hsa-miR320/LNA_PM | tTcgmCccTctmCaamCccAgcTttt |
| hsa-miR26a/LNA_PM | agcmCtaTccTggAttActTgaa |
| hsa-miR99a/LNA_PM | cacAagAtcGgaTctAcgGgtt |
| hsa-miR15a/LNA_PM | cAcaAacmCatTatGtgmCtgmCta |
| hsa-miR16-1/LNA_PM | cgmCcaAtaTttAcgTgcTgcTa |
| hsa-miR24/LNA_PM | cTgtTccTgcTgaActGagmCca |
| hsa-let7g/LNA_PM | amCtgTacAaamCtamCtamCctmCa |
| hsa-let7a/LNA_MM | aamCtaTacAacAtamCtamCctmCa |
| hsa-let7f/LNA_MM | aamCtaTacAatAtamCtamCctmCa |
| hsa-miR143LNA_MM | tGagmCtamCagmCgcTtcAtcTca |
| hsa-miR145/LNA_MM | aAggGatTccTcgGaaAacTggAc |
| hsa-miR320/LNA_MM | tTcgmCccTctAaamCccAgcTttt |
| hsa-miR26a/LNA_MM | agcmCtaTccTcgAttActTgaa |
| hsa-miR99a/LNA_MM | cacAagAtcGcaTctAcgGgtt |
| hsa-miR15a/LNA_MM | cAcaAacmCatmCatGtgmCtgmCta |
| hsa-miR16-1/LNA_MM | cgmCcaAtaTttTcgTgcTgcTa |
| hsa-miR24/LNA_MM | cTgtTccTgcmCgaActGagmCca |
| hsa-let7g/LNA_MM | amCtgTacAaaAtamCtamCctmCa |

### Example 11

### List of LNA-substituted Detection Probes for Expression Profiling of Human and Mouse microRNAs by Oligonucleotide Microarrays

LNA nucleotides are depicted by capital letters, DNA nucleotides by lowercase letters, mC denotes LNA methyl-cytosine, PM perfect match to the miRNA, MM one mismatch at the central position of the probe sequence, dir denotes the probe sequence corresponding to the mature miRNA sequence, rev denotes the probe sequence complementary to the mature miRNA sequence in question. The detection probes can be used t as capture sequences for expression profiling by LNA oligonucleotide microarrays. Covalent attachment to the solid surfaces of the capture probes can be accomplished by incorporating a NH2-C6- or a NH2-C6-hexaethylene glycol monomer or dimer group at the 5′-end or at the 3′-end of the probes during synthesis.

| | | Self- |
|---|---|---|
| | | comp |
| Probe name | Sequence 5′-3′ | score |
| mmu-let7adirPM/LNA | tgaGgtAgtAggTtgTatA | 30 |
| | gtt | |
| mmu-miR1dirPM/LNA | tgGaaTgtAaaGaaGtaTg | 18 |
| | ta | |
| mmu-miR16dirPM/LNA | tagmCagmCacGtaAatAt | 46 |
| | tGgcg | |
| mmu-miR22dirPM/LNA | aagmCtgmCcaGttGaaGa | 48 |
| | amCtgt | |
| mmu-miR26bdirPM/LNA | tTcaAgtAatTcaGgaTag | 35 |
| | Gtt | |
| mmu-miR30cdirPM/LNA | tgtAaamCatmCctAcamC | 27 |
| | tcTcaGc | |
| mmu-miR122adirPM/LNA | tggAgtGtgAcaAtgGtgT | 32 |
| | ttg | |
| mmu-miR126stardirPM/LNA | catTatTacTttTggTacG | 28 |
| | cg | |
| mmu-miR126dirPM/LNA | tcgTacmCgtGagTaaTaa | 32 |
| | Tgc | |
| mmu-miR133dirPM/LNA | tTggTccmCctTcaAccAg | 37 |
| | cTgt | |
| mmu-miR143dirPM/LNA | tGagAtgAagmCacTgtAg | 49 |
| | cTca | |
| mmu-miR144dirPM/LNA | tAcaGtaTagAtgAtgTac | 41 |
| | Tag | |
| mmu-let7arevPM/LNA | aamCtaTacAacmCtamCt | 16 |
| | amCctmCa | |
| mmu-miR1revPM/LNA | tamCatActTctTtamCat | 11 |
| | Tcca | |
| mmu-miR16revPM/LNA | cgmCcaAtaTttAcgTgcT | 34 |
| | gcTa | |
| mmu-miR22revPM/LNA | acaGttmCttmCaamCtgG | 48 |
| | caGctt | |
| mmu-miR26brevPM/LNA | aacmCtaTccTgaAttAct | 28 |
| | Tgaa | |
| mmu-miR30crevPM/LNA | gmCtgAgaGtgTagGatGt | 33 |
| | tTaca | |
| mmu-miR122arevPM/LNA | cAaamCacmCatTgtmCac | 25 |
| | ActmCca | |
| mmu-miR126starrevPM/LNA | cgmCgtAccAaaAgtAatA | 28 |
| | atg | |
| mmu-miR126revPM/LNA | gcAttAttActmCacGgtA | 25 |
| | cga | |
| mmu-miR133revPM/LNA | acAgcTggTtgAagGggAc | 41 |
| | cAa | |
| mmu-miR143revPM/LNA | tGagmCtamCagTgcTtcA | 56 |
| | tcTca | |
| mmu-miR144revPM/LNA | ctaGtamCatmCatmCtaT | 37 |
| | acTgta | |
| mmu-let7adirMM/LNA | tgaGgtAgtAagTtgTatA | 34 |
| | gtt | |
| mmu-miR1dirMM/LNA | tgGaaTgtAagGaaGtaTg | 18 |
| | ta | |
| mmu-miR16dirMM/LNA | tAgcAgcAcgGaaAtaTtg | 33 |
| | Gcg | |
| mmu-miR22dirMM/LNA | aaGctGccAggTgaAgaAc | 35 |
| | tGt | |
| mmu-miR26bdirMM/LNA | tTcaAgtAatGcaGgaTag | 27 |
| | Gtt | |
| mmu-miR30cdirMM/LNA | tgtAaamCatmCatAcamC | 27 |
| | tcTcaGc | |
| mmu-miR122adirMM/LNA | tggAgtGtgAaaAtgGtgT | 29 |
| | ttg | |
| mmu-miR126stardirMM/LNA | catTatTacTgtTggTacG | 35 |
| | cg | |
| mmu-miR126dirMM/LNA | tmCgtAccGtgGgtAatAa | 39 |
| | tGc | |
| mmu-miR133dirMM/LNA | ttgGtcmCccTgcAacmCa | 42 |
| | gmCtgt | |
| mmu-miR143dirMM/LNA | tGagAtgAagAacTgtAgc | 49 |
| | Tca | |
| mmu-miR144dirMM/LNA | tAcaGtaTagGtgAtgTac | 41 |
| | Tag | |
| mmu-let7arevMM/LNA | aActAtamCaamCttActA | 17 |
| | ccTca | |
| mmu-miR1revMM/LNA | tacAtamCttmCctTacAt | 11 |
| | tmCca | |
| mmu-miR16revMM/LNA | cgmCcaAtaTttmCcgTgc | 34 |
| | TgcTa | |
| mmu-miR22revMM/LNA | amCagTtcTtcAccTggmC | 35 |
| | agmCtt | |
| mmu-miR26brevMM/LNA | aamCctAtcmCtgmCatTa | 24 |
| | cTtgAa | |
| mmu-miR30crevMM/LNA | gmCtgAgaGtgTatGatGt | 29 |
| | tTaca | |
| mmu-miR122arevMM/LNA | cAaamCacmCatTttmCac | 13 |
| | ActmCca | |
| mmu-miR126starrevMM/LNA | cgmCgtAccAacAgtAatA | 31 |
| | atg | |
| mmu-miR126revMM/LNA | gmCatTatTacmCcamCgg | 39 |
| | TacGa | |
| mmu-miR133revMM/LNA | acaGctGgtTgcAggGgam | 45 |
| | Ccaa | |
| mmu-miR143revMM/LNA | tgAgcTacAgtTctTcaTc | 49 |
| | tmCa | |
| mmu-miR144revMM/LNA | ctAgtAcaTcamCctAtam | 31 |
| | CtgTa | |

### Example 12

### A Loss of Function Assay for miRNAs

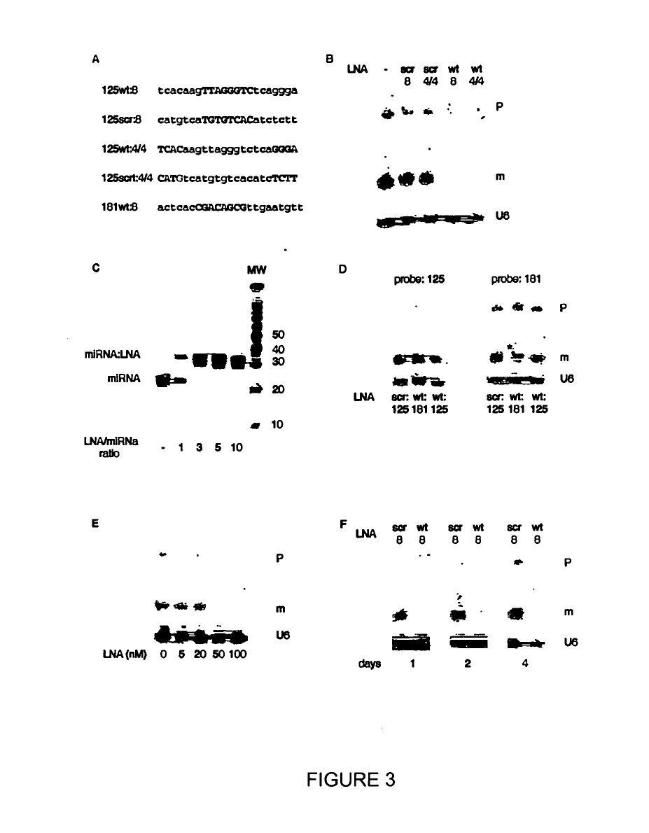

In order to address miRNA function in myoblastic differentiation, we designed a loss-of-function assay based on antisense oligonucleotides complementary to the miRNA sequence. Antisense oligonucleotides have been used to demonstrate a function for miRNAs in drosophila (Boutla et al., 2003). Antisense oligonucleotides with a 2′-O-methyl modification have been shown to block RISC activity (Hutvagner et al., 2004; Meister et al., 2004). We chose to use oligonucleotides containing locked nucleotides (LNAs) (Kumar et al., 1998). These form highly stable duplexes with complementary RNAs (Koshkin, 1998), even when using short sequences (Kurreck et al., 2002). They trigger the degradation of target mRNAs by RNase H, provided that the locked nucleotides are not located at the center of the sequence. (Wahlestedt et al., 2000). A stable interaction between the antisense oligonucleotide and the miRNA would be expected to facilitate the sequestration of the miRNA in a duplex unable to interact with cellular protein complexes such as RISC or the miRNPs, or to anneal with complementary target mRNAs. Alternatively, it could induce the degradation of the miRNA by RNase H, similar to what was described for LNA/DNA oligonucleotides targetting mRNAs. Mixed LNA/DNA oligonucleotides with LNAs located either at the center or at the extremities of the sequence were transfected into myoblatsts, and mRNAs were analyzed by northern blotting. Results (FIG. 3B) showed that the target miRNA became undetectable (FIG. 3B) in cells transfected with LNA/DNA antisense molecules, but not in cells transfected with a control sequence. This effect was independent of the position of the locked nucleotides, whether in the center (wt125:8) or at the ends (wt125:4/4) of the antisense oligonucleotides. Thus it is unlikely that the LNA induces miRNA cleavage by RNase H.

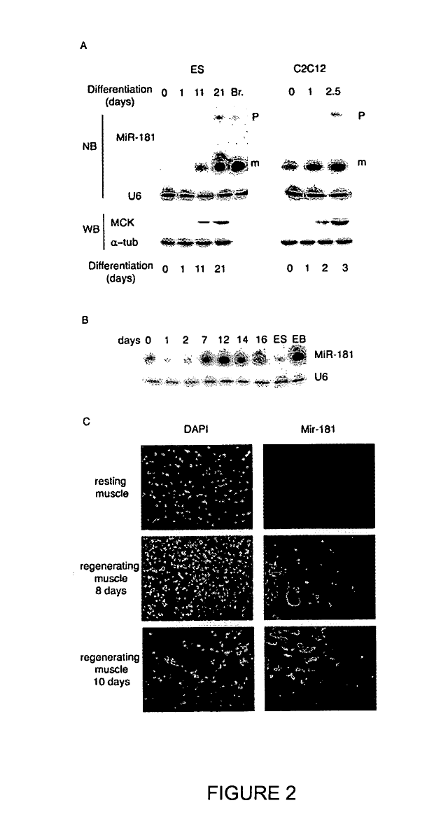

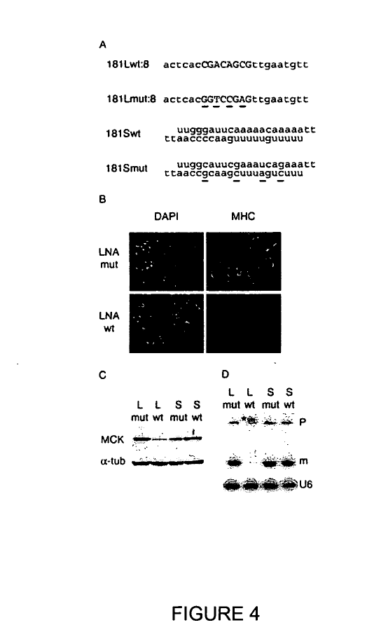

We favour a hypothesis in which the LNA/DNA oligonucleotides form a highly stable duplex with the miRNA, that subsequently resists the denaturing conditions used for northern blot, thereby preventing microRNA detection. Supporting this hypothesis, in some experiments we observed a faint smear migrating above the miRNA position that might correspond to melting miRNA/LNA duplexes (see for example FIG. 3D or FIG. 4D). Furthermore, a duplex formed in vitro between a radiolabelled synthetic miRNA and the 125wt:8 LNA indeed resisted the denaturing conditions of the migration (7 M Urea), with about 50% of the miRNA annealed to the LNA at a 1 to 1 ration of LNA to miRNA, and 100% at a 3 to 1 ratio (FIG. 3C). In extracts from transfected cells, the LNA also affected the apparent level of precursor (FIG. 2B). This effect, however, was only partial, as compared to the reduction observed with the mature miRNA. This might be due to the fact that the target sequence is more accessible in the mature miRNA than in the precursor, the latter having a secondary structure a priori more difficult to invade by the LNA. Alternatively, the LNA might influence the actual level of precursor by affecting a regulatory loop in miRNA metabolism. The effect was sequence specific and was not observed with a scrambled oligonucleotide (FIG. 3A and B). Furthermore, inhibiting one miRNA did not affect another miRNA: LNA/DNA 10 complementary to miR-125b affected miR-125b but did not have any effect on miR-181 and, conversely, LNA/DNA complementary to miR-181 affected miR-181 but had no effect on miR- 125b (FIG. 3D). A dose response analysis of the LNA/DNA oligonucleotide showed that the effect was dose dependent, and that 50 nM of LNA/DNA were sufficient to affect the target miRNA (FIG. 3E). Finally, LNA-containing oligonucleotides are highly stable in physiological media (Kurreck et al., 2002). Consistent with this observation, inhibition of target miRNAs lasted for at least several days in myoblastic cells (FIG. 3F), and the LNA persisted for the same duration (FIG. 7), a timing compatible with the functional analysis of miRNAs during in vitro differentiation of myoblastic cells. Taken together, these data show that this approach is usable to address miRNA functions during the differentiation process.

MiR-181 is Required for Muscle Cell Terminal Differentiation

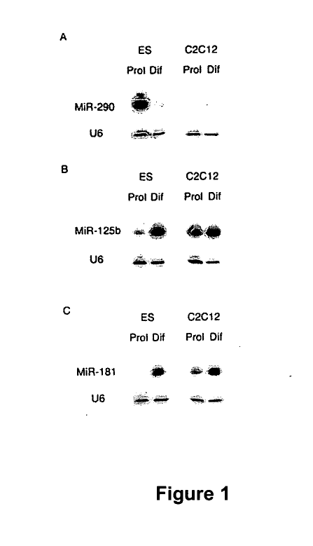

MiR-181 has been described as having a role in B lymphocytes, and its upregulation during skeletal muscle differentiation ex vivo and during muscle regeneration in vivo (FIG. 1 and 2) was unexpected. In order to analyze its function, C2C12 cells were treated with the LNA/DNA oligonucleotide complementary to miR-181, or with a mutated sequence as a control (FIG. 4A), and myotube formation and muscle marker expression were monitored. Cells treated with miR- 181 LNA oligonucleotide formed few myotubes (FIG. 4B) and expressed low levels of muscle specific markers such as MHC (FIG. 4B) or MCK (FIG. 4C), as compared to cells treated with mutant LNA. In fact, most of the cells scoring positive for MHC in the miR-181 LNA-treated population were neither elongated nor fused to form multinucleated myotubes (FIG. 4B). An siRNA directed against the precursor loop of miR-181 (FIG. 4A), and designed as previously described (Zeng et al., 2002), did not affect differentiation (FIG. 4 B and C). Monitoring miR- 181 expression, however, revealed that, in contrast to the LNA, the siRNA did not significantly decrease the levels of mature or precursor miR-181 (FIG. 4D). This lack of activity might be due to the remarkably AT-rich nature of the target loop. Alternatively, it might be due to the apparent long half-life of miRNA in these cells: indeed, down-regulating DICER, the enzyme involved in precursor processing, resulted in increased levels of precursor but not in reduced levels of mature miRNA (FIG. 7).

Inhibition is Rescued by miR-181

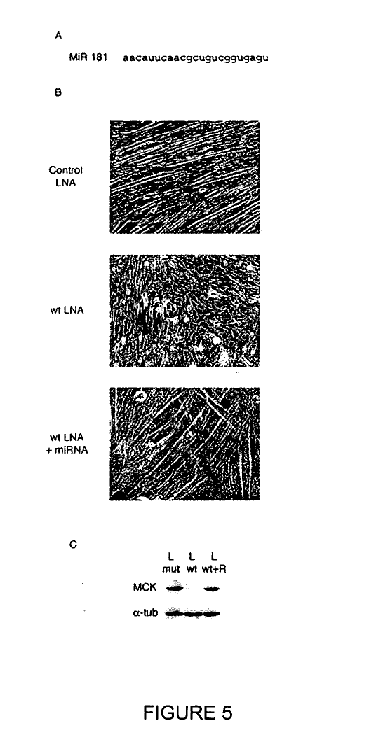

The level of expression achieved with a plasmid designed as previously described (Zeng et al., 2002) was very low. Rescue experiments were therefore performed by co-transfecting the miRNA as a synthetic sequence. When cotransfected with the LNA, synthetic miR-181 was able to restore a significant level of differentiation in transfected cells, as assessed by the number of myotubes (FIG. 5B) and the expression of muscle markers. The miRNA alone did not affect differentiation. Furthermore, differentiation was not restored by cotransfection of the LNA with an irrelevant RNA sequence. Taken together, these results demonstrate a sequence-specific effect of the LNA, and rule out non-specific effects potentially induced by the formation of an LNA/RNA duplex in the cells.

Mir181 and Hox-a11 are in the Same Genetic Pathway

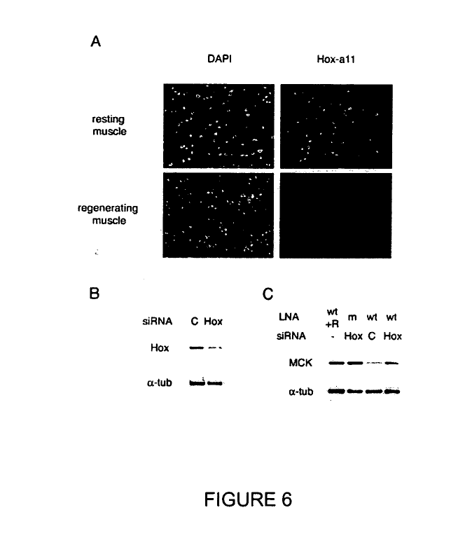

In mammals, miRNA targets have been predicted in silico (Lewis et al., 2003), but have been validated in a very limited number of cases. Among the potential targets identified in silico for miR181, some may be relevant to muscle. In particular, Hoxa11, a homeodomain protein that is essential for limb (Small and Polter, 1993) and kidney formation (Patterson et al., 2001), is a repressor of MyoD function (Yamamoto and Kuroiwa, 2003). Immunofluorescence analysis demonstrated that it is expressed in nuclei of adult resting muscle, confirming previous observations (Takahashi et al., 2004), but is undetectable in regenerating muscle (FIG. 6A), when miR181 expression is upregulated. In order to determine whether Hox-a11 could be a target for miR181 in muscle, we used a synthetic siRNA to knock down the protein (FIG. 6B). We reasoned that if Hox-a11 is downstream of miR181, then co-inhibiting miR181 and Hox-a11 simultaneously should restore, at least in part, normal differentiation. Indeed, whereas a control siRNA had no effect on MCK expression, the siRNA against Hox-a11 significantly rescued the differentiation phenotype (FIG. 6C). Thus, knocking down Hox-a11 resulted in what might be thought of as a phenotypic suppressive mutation. This results indicates that Hox-a11 is in the same genetic pathway as miR181, and supports a model in which Hox-a11 is a direct target of this miRNA.

Experimental Procedures

Cell culture and transfection

Myoblastic C2C12 cells were maintained in Dulbecco's minimal essential medium (DMEM), (Life Technologies, Inc.) supplemented with 15% fetal bovine serum (Dominique Dutscher). For transfections, 5×10⁴ cells were plated in 6-well plates and transfected the next day with 1 μg of oligonucleotides using Lipofectamine (Invitrogen). To induce terminal differentiation, 24 h after transfection, growth medium was replaced by differentiation medium (DMEM, 2% horse serum, Sigma). Phenotypic differentiation was observed after 72-96 h.

Mouse ES cells (kind gift or Dr. Muriel Vernet) were cultured and maintained in an undifferentiated state as described (Zhuang et al., 1992). For embryoid body (EB) formation they were diluted and kept in suspension for 4 days. To induce muscle differentiation EB were maintained an additional 2 days in presence of 1% DMSO in DMEM with 10% horse serum, then plated on tissue culture dishes in DMEM with 2% horse serum. Myotubes were observed at 21 days of differentiation.

Cardiotoxin-induced Regeneration Assay

6 to 7 week-old Balb/c mice were put to sleep with Avertin as described in (Weiss and Zimmermann, 1999), the leg was shaved, and injections were made into the tibialis anterior (TA) muscle, using a 29 G ½ insulin syringe. Cardiotoxin (Latoxan) was diluted to 10 μM in PBS, and 25 μl were injected per TA muscle (Hosaka et al., 2002). Control animals received equal volumes of PBS. TA muscles were harvested at different time points, and total RNA was extracted and used for northern blot.

Oligonucleotides

SiRNA, RNA, DNA and LNA/DNA mixed oligonucleotides were obtained from Proligo, France.

Northern blot

For northern blot analysis 30 μg of total RNA were separated in 15% denaturating polyacrylamide gels and electro-transferred to Hybond-N+ membranes. DNA anti-sense probes were end-labeled with T4 polynucleotide kinase (BioLabs) using y-32P-ATP with high specific activity (7000 Ci/mmol, ICN). Hybridization and washing were carried out in Rapid-hyb buffer (Amersham) at 42° C. U6 expression was tested as loading control.

In situ hybridization

The miRNAin situ hybridization protocol by Ambion was optimized for skeletal muscle. Briefly, the tibialis anterior (TA) muscle was frozen in Tissue-Tek OCT reagent, cryo-sections (12 μM) were prepared, de-proteinized and acetylated as described in (DeNardi et al., 1993). Digoxigenin (DIG)—labeled miRNA probes (miR181: sequence complementary to the mature miRNA) were prepared according to the instructions for the mirVana Probe Construction kit (Ambion). Muscle sections were incubated with specific miRNA probes (500 ng/ml) overnight at 380° C., washed with 2XSSC at 38° C. for 20 min, and treated with 400 unit/ml (10 μg/ml) RNase A at 37° C. for 30 min. After two washes with PBS, the slides were incubated with FITC-coupled anti-DIG antibody (Roche) for 4 hours at room temperature, washed, rinsed with 200 ng/ml Hoecsht 33258 in PBS, mounted on glass slides with VectaShield (BioValley), and analyzed by fluorescent microscopy.

Wertern blot and Immunofluorescence

Western blot and immunostaining were performed as described previously (Polesskaya et al., 2001). Rabbit anti-MCK was kindly provided by Dr. Hidenory Ito, anti Hox-a11 was a kind gift of Larry Patterson, anti-MHC (MY-32) and Cy-3 conjugated anti-mouse IgG were purchased from Sigma.

In-vitro analysis of miRNA/LNA duplexes

Synthetic miR-181a RNA was end-labeled with 32P-ATP using T4 polynucleotide kinase, purified on a G-25 column and mixed with cold miR-181 so that the final activity was 105 cpm/pmol RNA. 10 pmol of labeled miR-181 were mixed with increasing amounts of LNA oligonucleotides in a total volume of 20 μl of DEPC-treated water containing 1 U/μl Rnase Inhibitor (Life Technologies). After 15 min incubation at room temperature, 10 μ1 of the olignucleotide mixture was mixed with 2×Urea loading buffer, incubated 15 min at 65° C. and separated on a 15% denaturating polyacrylamide gel. RNAs were electro-transferred to Hybond19 N+ membranes. Free RNA and complexes were detected by autoradiography. The 32P-labeled molecular weight ladder was purchased from Ambion.

TABLE I
Expression of various miRNAs during muscle differentiation.

| MiRNA | ES | EB | Myoblasts | Myotubes |
|---|---|---|---|---|
| 172 | +/− | + | +/− | +/− |
| 296 | +/− | + | +/− | +/− |
| 298 | +/− | + | +/− | +/− |
| 300 | +/− | ++ | − | − |
| 290 | ++ | − | − | − |
| 291s | ++ | − | − | − |
| 291as | ++ | − | − | − |
| 295 | ++ | − | − | − |
| 299 | ++ | +/− | +/− | +/− |
| 16 | − | + | +/− | +/− |
| 21 | − | ++ | ++ | ++ |
| 22 | +/− | + | ++ | ++ |
| 99a | − | nd | +/− | +/− |
| 125b | +/− | ++ | ++ | ++ |
| 143 | +/− | ++ | ++ | ++ |
| let7d | − | nd | + | + |
| let7i | − | nd | + | + |
| let7g | − | nd | + | + |
| 133 | − | ++ | − | + |
| 181 | − | ++ | +/− | ++ |
| 208 | − | + | − | + |
| 297 | − | + | − | + |
| let7c | − | + | +/− | ++ |
| 10b | − | − | − | − |
| 15a | − | − | − | − |
| 15b | − | − | − | − |
| 99b | − | − | − | − |
| 106 | − | − | − | − |
| 129 | − | − | − | − |
| 131 | − | − | − | − |
| 142s | − | − | − | − |
| 142as | − | − | − | − |
| 213 | − | − | − | − |
| 302 | − | − | − | − |

Expression of miRNAs were analyzed by northern blot in ES cells, either proliferating (ES) or forming muscle-oriented embryonic bodies (EB), as well as in C212 cells, either proliferating (Myoblasts) or differentiated into myotubes (Myotubes).
MiRNAs are clustered according to their expression profile.
Expression levels:
high: ++;
medium: +;
low: +/−;
undetectable: −;
nd: not done

References

Ambros, V. (2001). microRNAs: tiny regulators with great potential. Cell 107, 823-826.

Ambros, V. (2003). MicroRNA pathways in flies and worms: growth, death, fat, stress, and timing. Cell 113, 673-676.

Aravin, A. A., Lagos-Quintana, M., Yalcin, A., Zavolan, M., Marks, D., Snyder, B., Gaasterland, T., Meyer, J., and Tuschl, T. (2003). The small RNA profile during Drosophila melanogaster development. Dev Cell 5, 337-350.

Bains, W., Ponte, P., Blau, H., and Kedes, L. (1984). Cardiac actin is the major actin gene product in skeletal muscle cell differentiation in vitro. Mol Cell Biol 4, 1449-1453.

Boutla, A., Delidakis, C., and abler, M. (2003). Developmental defects by antisense-mediated inactivation of micro-RNAs 2 and 13 in Drosophila and the identification of putative target genes. Nucleic Acids Res 31, 4973-4980.

Brennecke, J., Hipfner, D. R., Stark, A., Russell, R. B., and Cohen, S. M. (2003). bantam encodes a developmentally regulated microRNA that controls cell proliferation and regulates the proapoptotic gene hid in Drosophila. Cell 113, 25-36.

Buckingham, M. (2001). Skeletal muscle formation in vertebrates. Curr Opin Genet Dev 11, 440-448.

Carrington, J. C., and Ambros, V. (2003). Role of microRNAs in plant and animal development. Science 301, 336-338.

Charge, S. B., and Rudnicki, M. A. (2004). Cellular and molecular regulation of muscle regeneration. Physiol Rev 84, 209-238.

Chen, C. Z., Li, L., Lodish, H. F., and Bartel, D. P. (2004). MicroRNAs modulate hematopoietic lineage differentiation. Science 303, 83-86.

Chen, F., Cooney, A. J., Wang, Y., Law, S. W., and O'Malley, B. W. (1994). Cloning of a novel orphan receptor (GCNF) expressed during germ cell development. Mol Endocrinol 8, 1434-1444.

DeNardi, C., Ausoni, S., Moretti, P., Gorza, L., Velleca, M., Buckingham, M., and Schiaffino, S. (1993). Type 2X-myosin heavy chain is coded by a muscle fiber type-specific and developmentally regulated gene. J Cell Biol 123, 823-835.

Dinsmore, J., Ratliff, J., Deacon, T., Pakzaban, P., Jacoby, D., Galpern, W., and Isacson, O. (1996). Embryonic stem cells differentiated in vitro as a novel source of cells for transplantation. Cell Transplant 5, 131-143.

Finnegan, E. J., and Matzke, M. A. (2003). The small RNA world. J Cell Sci 116, 4689-4693.

Hake, S. (2003). MicroRNAs: a role in plant development. Curr Biol 13, R851-852.

Hosaka, Y., Yokota, T., Miyagoe-Suzuki, Y., Yuasa, K., Imamura, M., Matsuda, R., Ikemoto, T., Kameya, S., and Takeda, S. (2002). Alpha1-syntrophindeficient skeletal muscle exhibits hypertrophy and aberrant formation of neuromuscular junctions during regeneration. J Cell Biol 158,1097-1107.

Houbaviy, H. B., Murray, M. F., and Sharp, P. A. (2003). Embryonic stem cell-specific MicroRNAs. Dev Cell 5, 351-358.

Hummelke, G. C., and Cooney, A. J. (2001). Germ cell nuclear factor is a transcriptional repressor essential for embryonic development. Front Biosci 6, Dl 186-1191.

Hutvagner, G., Simard, M. J., Mello, C., and Zamore, P. (2004). Sequence-Specific Inhibition of Small RNA Function. PLOS Biology 2, 1-11.

Kole, R., and Sazani, P. (2001). Antisense effects in the cell nucleus: modification of splicing. Curr Opin Mol Ther 3, 229-234.

Koshkin, A. A., Nielsen, P., Meldgaard, M., Rajwanshi, V. K., Singh, S. K. & Wengel, J (1998). J Am Chem Soc 120, 13252-13260.

Krichevsky, A. M., King, K. S., Donahue, C. P., Khrapko, K., and Kosik, K. S. (2003). A microRNA array reveals extensive regulation of microRNAs during brain development. Rna 9, 1274-1281.

Kumar, R., Singh, S. K., Koshkin, A. A., Rajwanshi, V. K., Meldgaard, M., and Wengel, J. (1998). The first analogues of LNA (locked nucleic acids): phosphorothioate-LNA and 2′-thio-LNA. Bioorg Med Chem Lett 8, 2219-2222.

Kurreck, J., Wyszko, E., Gillen, C., and Erdmann, V. A. (2002). Design of antisense oligonucleotides stabilized by locked nucleic acids. Nucleic Acids Res 30, 1911-1918.

Lagos-Quintana, M., Rauhut, R., Meyer, J., Borkhardt, A., and Tuschl, T. (2003). New microRNAs from mouse and human. Rna 9, 175-179.

Lagos-Quintana, M., Rauhut, R., Yalcin, A., Meyer, J., Lendeckel, W., and Tuschl, T. (2002). Identification of tissue-specific microRNAs from mouse. Curr Biol 12, 735-739.

Lee, Y., Jeon, K., Lee, J. T., Kim, S., and Kim, V. N. (2002). MicroRNA maturation: stepwise processing and subcellular localization. Embo J 21, 4663-4670.

Lewis, B. P., Shih, l. H., Jones-Rhoades, M. W., Bartel, D. P., and Burge, C. B. (2003). Prediction of mammalian microRNA targets. Cell 115, 787-798.

Mallory, A. C., and Vaucheret, H. (2004). MicroRNAs: something important between the genes. Curr Opin Plant Biol 7, 120-125.

Meister, G., Landthaler, M., Dorsett, Y., and Tuschl, T. (2004). Sequence-specific inhibition of microRNA- and siRNA-induced RNA silencing. Rna 10, 544-550.

Nelson, P. T., Hatzigeorgiou, A. G., and Mourelatos, Z. (2004). miRNP:mRNA association in polyribosomes in a human neuronal cell line. Rna 10, 387-394.

Olsen, P. H., and Ambros, V. (1999). The lin4 regulatory RNA controls developmental timing in Caenorhabditis elegans by blocking LIN-14 protein synthesis after the initiation of translation. Dev Biol 216, 671-680.

Patterson, L. T., Pembaur, M., and Potter, S. S. (2001). Hoxa11 and Hoxd11 regulate branching morphogenesis of the ureteric bud in the developing kidney. Development 128, 2153-2161.

Polesskaya, A., Seale, P., and Rudnicki, M. A. (2003). Wnt signaling induces the myogenic specification of resident CD45+ adult stem cells during muscle regeneration. Cell 113, 841-852.

Reinhart, B. J., Slack, F. J., Basson, M., Pasquinelli, A. E., Bettinger, J. C., Rougvie, A. E.,

Horvitz, H. R., and Ruvkun, G. (2000). The 21-nucleotide let-7 RNA regulates developmental timing in Caenorhabditis elegans. Nature 403, 901-906.

Schramke, V., and Allshire, R. (2003). Hairpin RNAs and retrotransposon LTRs effect RNAi and chromatin-based gene silencing. Science 301, 1069-1074.

Seale, P., Polesskaya, A., and Rudnicki, M. A. (2003). Adult stem cell specification by Wnt signaling in muscle regeneration. Cell Cycle 2, 418-419.

Seitz, H., Youngson, N., Lin, S. P., Dalbert, S., Paulsen, M., Bachellerie, J. P., Ferguson-Smith, A. C., and Cavaille, J. (2003). Imprinted microRNA genes transcribed antisense to a reciprocally imprinted retrotransposon-like gene. Nat Genet 34, 261-262.

Sempere, L. F., Freemantle, S., Pitha-Rowe, I., Moss, E., Dmitrovsky, E., and Ambros, V. (2004). Expression profiling of mammalian microRNAs uncovers a subset of brain-expressed microRNAs with possible roles in murine and human neuronal differentiation. Genome Biol 5, R13.

Small, K. M., and Potter, S. S. (1993). Homeotic transformations and limb defects in Hox A11 mutant mice. Genes Dev 7, 2318-2328.

Taghon, T., Thys, K., De Smedt, M., Weerkamp, F., Staal, F. J., Plum, J., and Leclercq, G. (2003). Homeobox gene expression profile in human hematopoietic multipotent stem cells and T-cell progenitors: implications for human T-cell development. Leukemia 17, 1157-1163.

Takahashi, Y., Hamada, J., Murakawa, K., Takada, M., Tada, M., Nogami, I., Hayashi, N., Nakamori, S., Monden, M., Miyamoto, M., et al. (2004). Expression profiles of 39 HOX genes in normal human adult organs and anaplastic thyroid cancer cell lines by quantitative real-time RTPCR system. Exp Cell Res 293, 144-153.

Verdel, A., Jia, S., Gerber, S., Sugiyama, T., Gygi, S., Grewal, S. I., and Moazed, D. (2004). RNAi-mediated targeting of heterochromatin by the RITS complex. Science 303, 672-676.

Wahlestedt, C., Salmi, P., Good, L., Kela, J., Johnsson, T., Hokfelt, T., Broberger, C., Porreca, F., Lai, J., Ren, K., et al. (2000). Potent and nontoxic antisense oligonucleotides containing locked nucleic acids. Proc Natl Acad Sci U.S.A 97, 5633-5638.

Weiss, J., and Zimmermann, F. (1999). Tribromoethanol (Avertin) as an anaesthetic in mice. Lab Anim 33, 192-193.

Xu, P., Vemooy, S. Y., Guo, M., and Hay, B. A. (2003). The Drosophila microRNA Mir-14 suppresses cell death and is required for normal fat metabolism. Curr Biol 13, 790-795.

Yamamoto, M., and Kuroiwa, A. (2003). Hoxa-11 and Hoxa-13 are involved in repression of MyoD during limb muscle development. Dev Growth Differ 45, 485-498.

Yekta, S., Shih, I. H., and Bartel, D. P. (2004). MicroRNA-directed cleavage of HOXB8 mRNA. Science 304, 594-596.

Zeng, Y., Wagner, E. J., and Cullen, B. R. (2002). Both natural and designed micro RNAs can inhibit the expression of cognate mRNAs when expressed in human cells. Mol Cell 9, 1327-1333.

Zhuang, Y., Kim, C. G., Bartelmez, S., Cheng, P., Groudine, M., and Weintraub, H. (1992). Helixloop-helix transcription factors E12 and E47 are not essential for skeletal or cardiac myogenesis, erythropoiesis, chondrogenesis, or neurogenesis. Proc NatI Acad Sci USA 89, 12132-12136.

### Example 13

### The microRNA miR-181 Targets the Homeobox Protein Hox-A11 During Mammalian Myoblast Differentiation

Deciphering the mechanisms underlying skeletal muscle-cell differentiation in mammals is an important challenge. Cell differentiation involves complex pathways regulated at both transcriptional and post-transcriptional levels. Recent observations have revealed the importance of small (20-25 base pair) non-coding RNAs (microRNAs or miRNAs) that are expressed in both lower organisms¹ and in mammals^{2,3}. miRNAs modulate gene expression by affecting mRNA translation⁴ or stability⁵. In lower organisms, miRNAs are essential for cell differentiation during development⁶⁻⁹; some miRNAs are involved in maintenance of the differentiated state. Here, we show that miR-181, a microRNA that is strongly upregulated during differentiation, participates in establishing the muscle phenotype. Moreover, our results suggest that miR-181 downregulates the homeobox protein Hox-A11 (a repressor of the differentiation process), thus establishing a functional link between miR-181 and the complex process of mammalian skeletal-muscle differentiation. Therefore, miRNAs can be involved in the establishment of a differentiated phenotype—even when they are not expressed in the corresponding fully differentiated tissue.

Muscle precursors, or myoblasts, are derived from multipotent precursor cells and acquire the myoblastic phenotype during the first step of skeletal muscle differentiation. They subsequently exit from the cell cycle and enter terminal differentiation, fusing into large multinucleated myotubes and expressing muscle-specific marker proteins. Differentiation can be partially recapitulated in two in vitro models: using either totipotent embryonic stem cells oriented toward the muscle lineage¹⁰, or established myoblastic cell lines that enter the skeletal muscle differentiation pathway by default when they are deprived of growth factors.

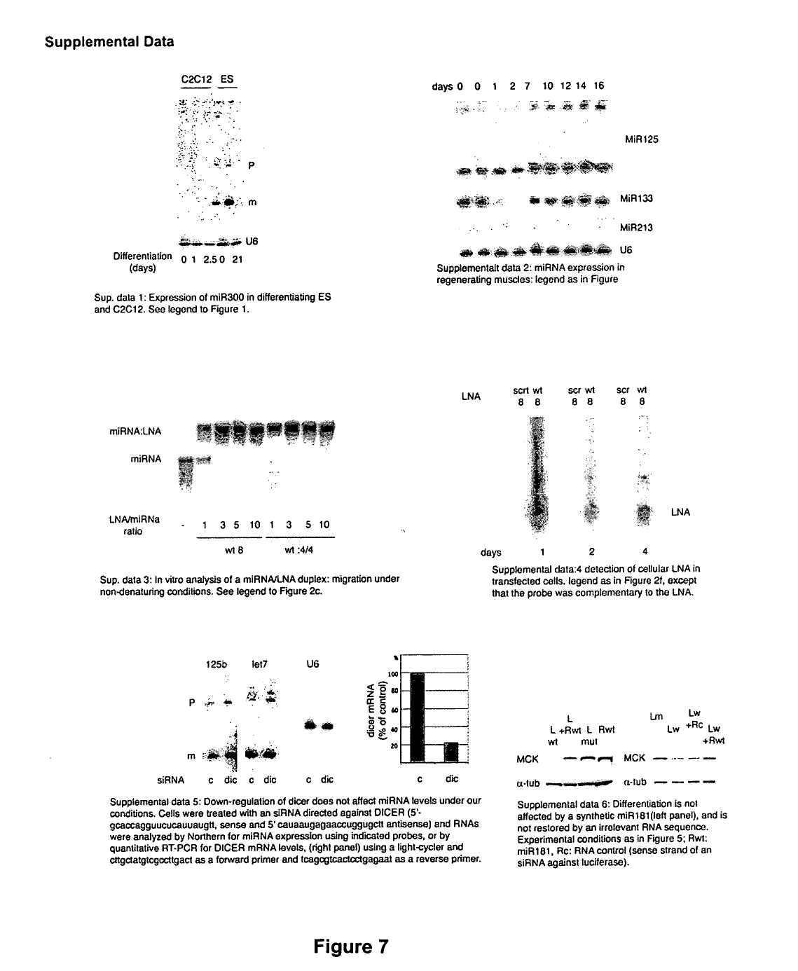

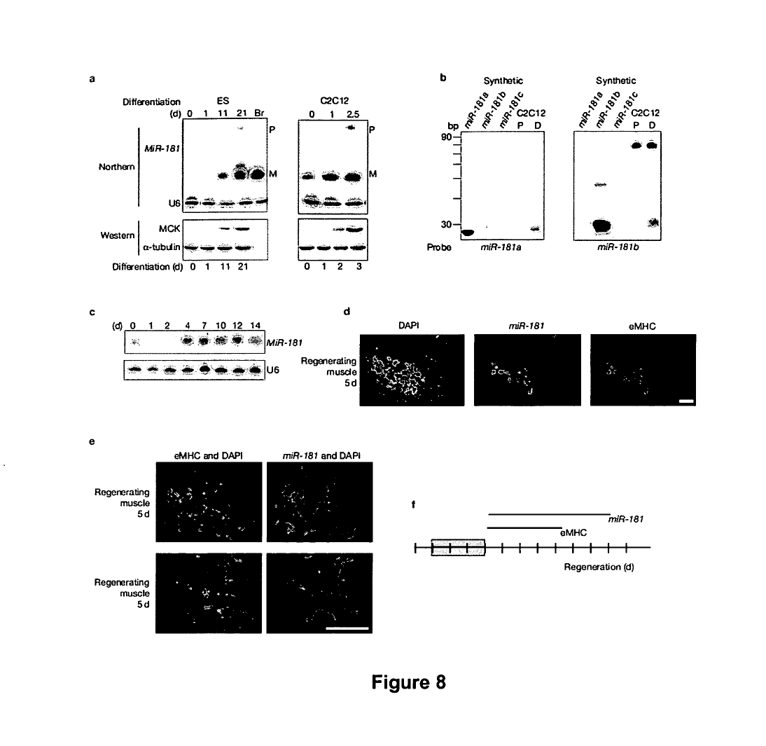

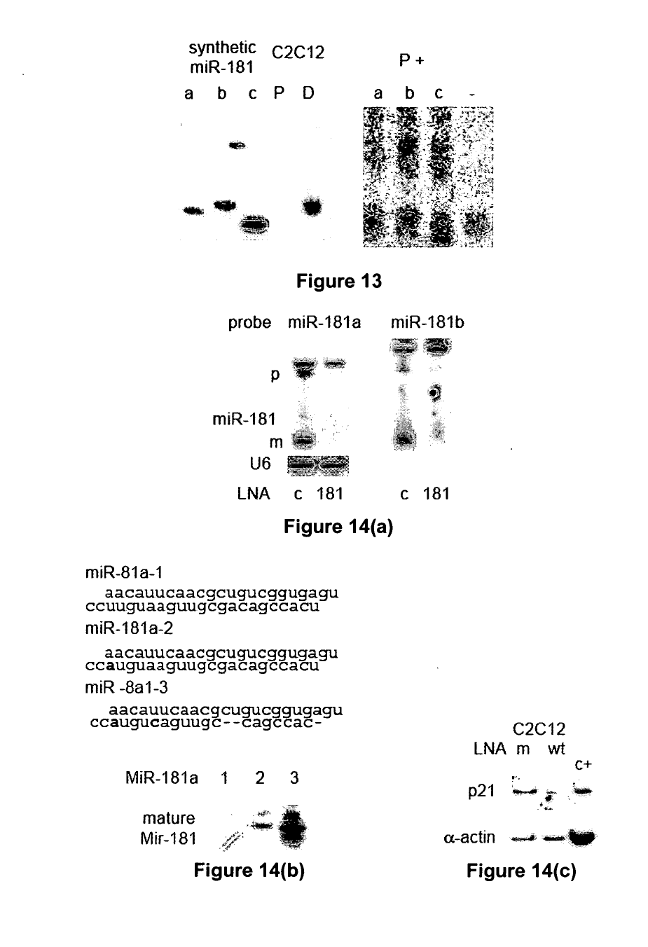

We screened for miRNAs that were differentially expressed during the two main steps of muscle differentiation and found that miR-181 is one of the most strongly upregulated miRNAs when terminal differentiation is induced in either of the two models. In myoblastic cells, miR-181 is upregulated before, or concomitant with, expression of differentiation-specific proteins such as muscle creatine kinase (MCK; FIG. 8a). Chemically modified locked nucleic acid (LNA) oligonucleotides¹¹ were used as probes to discriminate between the three previously characterized miR-181 iso-forms (hftp://microrna.sanger.ac.uk/), and miR-181a and miR-181b (FIG. 8b), but not miR-181c (FIG. 13) were detected. As one of the miR-181b precursors, miR-181b2, is located close to miR-181a on mouse chromosome 2, it is likely that the two isoforms are co-induced as a long primary transcript (pri-miRNA).

In vivo, miR-181 was barely detectable in the tibialis anterior muscle of adult mouse, as previously described^{9,12,} but was strongly upregulated on regeneration of muscle fibres (FIG. 8c). Cells expressing miR-181 were characterized by immunofluorescent in situ hybridization (FISH) experiments (FIG. 8d, e): miR-181 was detected by FISH, and embryonic myosin heavy chain (eMHC, a well-characterized protein marker of regenerating fibres) was detected by immunofluorescence microscopy. Nuclei were counterstained with DAPI to delineate the regeneration area (indicated by a high number of nuclei with a disorganized topology). miR-181-positive cells were mostly multinucleated in regenerating fibres, but some mononucleated cells were also labelled. Whether multinucleated or mononucleated, all of the miR-181-positive cells were also positive for eMHC, indicating that they were differentiating, nonproliferating muscle cells. Only about 80% of the cells positive for eMHC were also positive for miR-181. This can be explained by the sensitivities of the two detection assays: FISH uses such short probes that it is less sensitive than immunofluorescence microscopy. Although miR-181 returned to basal levels at the end of the regeneration process, it was still detectable for a long period after the disappearance of eMHC (FIG. 8f). miR-181 seems to have a long haHf-life in muscle cells, as suggested by its long-term (over several days) persistence in Dicer-depleted myoblastic cells, which may explain its long-term presence during regeneration.

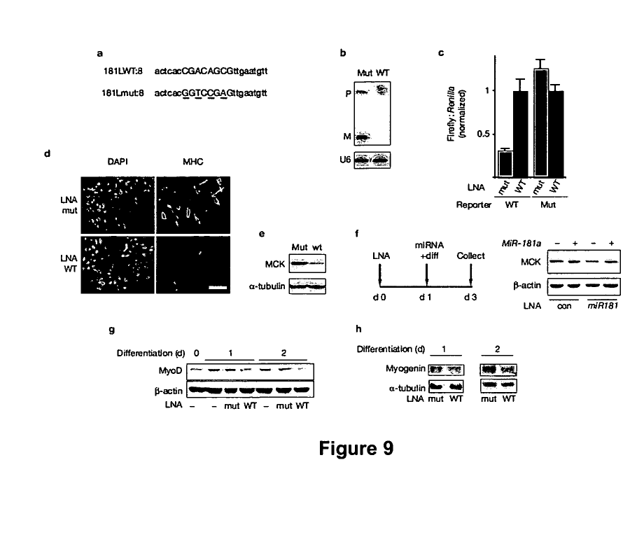

Together, these data indicate that miR-181, though poorty expressed in terminally differentiated muscle, is highly expressed in regenerating muscle, and raised the possibility that it may function during muscle differentiation. To address this issue, we designed an antisense-based loss-of-function assay^{3,14} using LNA-DNA-mixed-antisense oligonucleotides¹⁵: these oligonucleotides form highly stable sequence-specific duplexes with their target miRNA sequences, and are potent, specific and long-lasting inhibitors of these molecules. An LNA-DNA antisense oligonucleotide complementary to miR-181a (for sequence, see FIG. 9a) abrogated miR-181 detection (both a and b isoforms) in northern blots (FIG. 9b, FIG. 14a), most likely through sequestration of the target microRNA. miR-181 inhibition was also evident at a functional level, when a luciferase reporter construct harboring a sequence complementary to miR-181 was assayed. In C2C12 cells, the reporter activity was inhibited on differentiation. Inhibition was released by miR-181 antisense LNA, but not by a mutant form of the antisense oligonucleotide, whereas a reporter harbouring a mutated sequence was not affected (FIG. 9c). These results, along with similar data for a green fluorescent protein (GFP) target sequence, indicate that antisense LNAs can be used to analyse miRNA function in live cells.

The miR-181 antisense LNA oligonucleotides dramatically affected C2C12 myoblast differentiation, as assessed by both myotube formation and by the expression of sarcomeric myosin heavy chain (MHC; FIG. 9d) or MCK (FIG. 9e), both markers for terminal differentiation. A mutated antisense LNA did not affect differentiation-marker expression. Moreover, the differentiation phenotype was rescued by transfecting the miRNA as a synthetic double-stranded sequence (FIG. 9f), under conditions that result in high levels of cellular miRNA (monitored by northern blot, FIG. 14b). This demonstrated that the effect of the LNA-DNA antisense oligonucleotide was specifically due to miR-181 inhibition. The synthetic miRNA alone did not dramatically affect differentiation (FIG. 9f). Taken together, these data functionally link miR-181 to myoblast differentiation, even though miR-181 is barely detectable in resting muscle cells. This suggests that miR-181 is involved in the establishment of the differentiated phenotype, but probably not in its maintenance. Interestingly, during regeneration, a similar expression profile is observed for the transcription factors that orchestrate the differentiation process, the basic helix-loop-helix (bHLH) myogenic proteins MyoD and myogenin: these essential proteins are not detected in resting muscle and are expressed only on regeneration¹⁶.

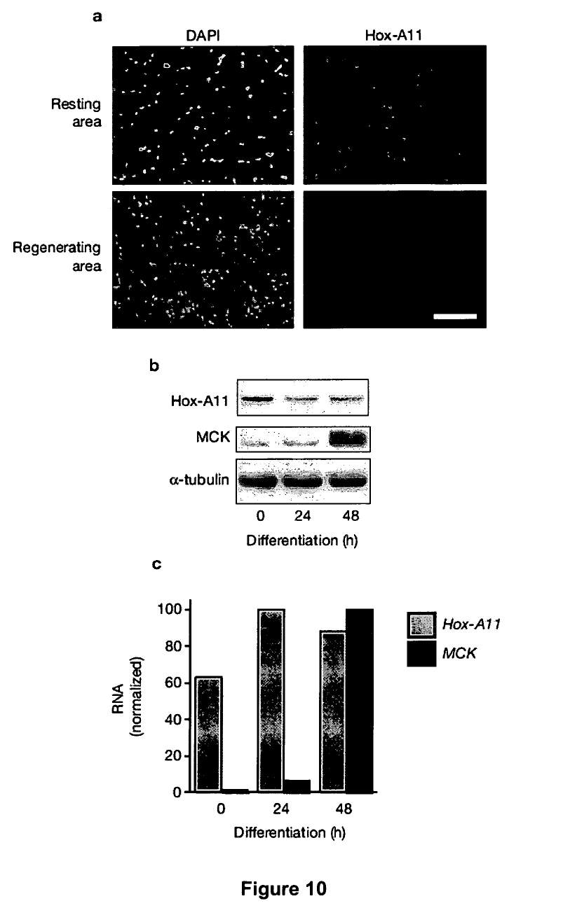

During differentiation of C2C12 cells in vitro, MyoD induces myogenin and triggers the entire differentiation programme. In miR-181- depleted C2C12 cells, MyoD expression was inhibited (FIG. 9g), as was the expression of its downstream targets, myogenin (FIG. 9h) and p21Cip1 (FIG. 14c). Therefore, miR-181 acts up-stream of MyoD in the differentiation pathway. One of the targets consistently predicted for miR-18117-19 (see hftp://www.microrna.org/ and hftp://pictar.bio.nyu.edu) is the homeobox protein Hox-A11. This protein is involved in urogenital tract development²⁰ but is also important for limb muscle patterning^{21,22} and can inhibit MyoD expression²³. In adult humans, Hox-A11 is detected in various organs, including skeletal muscle²⁴. The pattem of expression of Hox-A11 protein is complementary to that of miR-181 in muscle: Hox-A11 is highly expressed in cells with low miR-181 levels, resting muscle in vivo (FIG. 10a) and undifferentiated myoblasts in vitro (FIG. 10b), whereas Hox-A11 downregulation coincides with miRNA induction during terminal differentiation in both models (FIG. 10a, b).

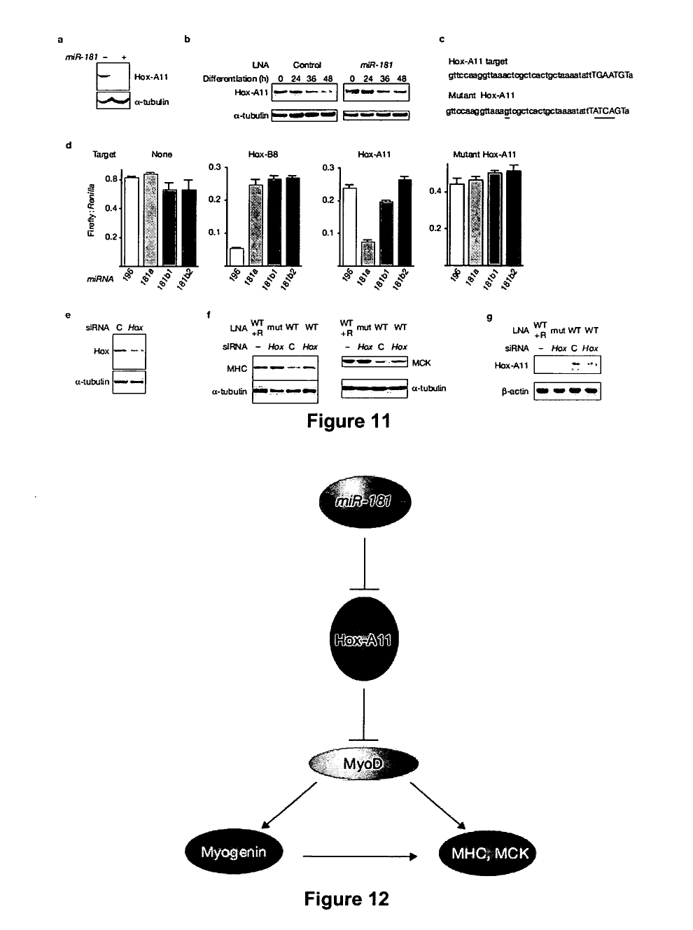

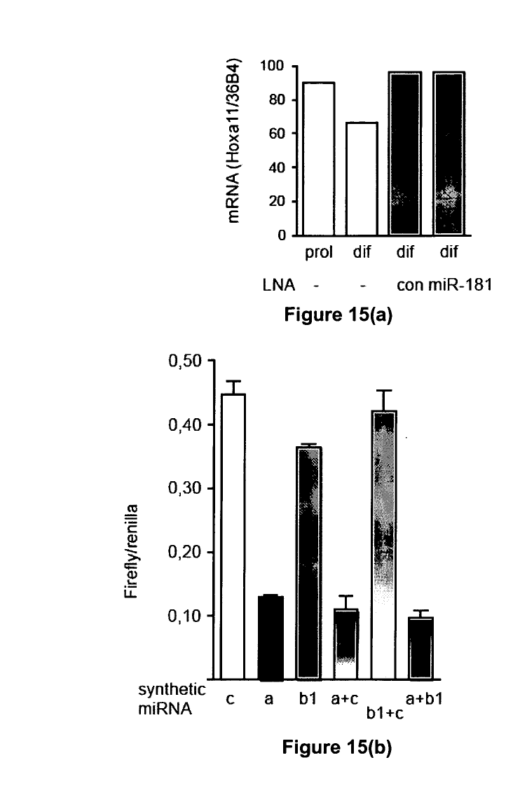

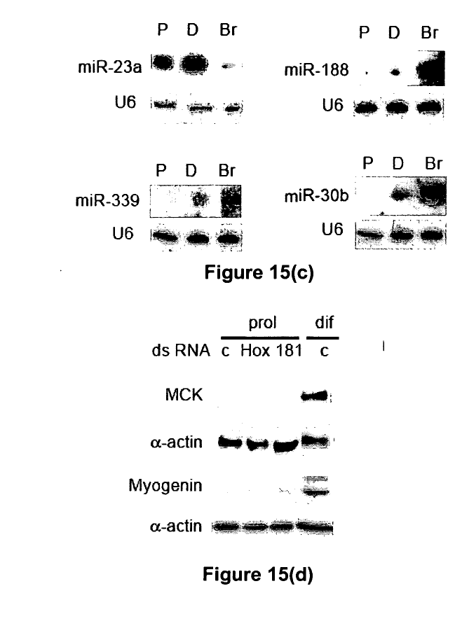

In differentiating C2C12 cells, despite reduced Hox-A11 protein levels, a concomitant decrease in Hox-A11 mRNA (FIG. 10c) was not observed, suggesting a post-transcriptional regulation mechanism. Whether or not miR-181 could affect Hox-A11 cellular protein levels was examined and it was found that Hox-A11 protein was downregulated by ectopic miR-181a in proliferating myoblasts (FIG. 11a). Conversely, Hox-A11 was upregulated by inhibition of miR-181; absolute levels of Hox-A11 protein were higher in cells treated with the LNA antisense oligonucleotides than in control cells (FIG. 11b). Twofold upregulation of the Hox- A11 protein was estimated by semi-quantitative analysis of the western blots, similarly to previously published data on miR-375 and its target, myotrophin²⁵. The LNA-DNA antisense oligonucleotide did not, however, abolish Hox-A11 downregulation on differentiation, suggesting that there are additional mechanisms of regulation. The level of Hox- A11 mRNA was not affected by inhibition of miR-181 with the LNA antisense oligonucleotide (FIG. 15a), even though the protein level increased, supporting a post-transcriptional mechanism. These data are consistent with the hypothesis that Hox-A11 is a direct target of miR-181 in muscle cells. To test this hypothesis, a standard reporter assay in miR-181-negative cells, using plasmids with tandem repeats of Hox-A11 predicted target sequences (FIG. 11c) inserted downstream of the firefly luciferase gene, was used. Insertion of wild-type sequences rendered the reporter sensitive to ectopic miR- 181a (FIG. 11d). Mutation of the target sequences abolished this effect. Moreover, a reporter harbouring an irrelevant target sequence (miR- 196 target sequence from Hox-B8)⁵ was not affected by miR-181a, and was inhibited only by its cognate microRNA, miR-196. Interestingly, the effect on the Hox-A11 target sequence was far more pronounced with miR-181a than with miR-181b. Moreover, co-transfection of the miR-181b isoform did not increase inhibition by the miR-181a isoform in the luciferase assay (FIG. 15b). In the literature, miR-181 is consistently predicted to bind to Hox-A11 mRNA, although with differing scores; the best-ranked isoform varies between different studies. Our experimental data best fit with the predictions that identified the a isoform of miR-181 as a miRNA that potentially binds to Hox-A11 (refs 17-19).

Taken together, our data indicate that Hox-A11 is a direct target of miR- 181 during mammalian muscle differentiation. This was confirmed at the functional level by simultaneously inhibiting miR-181 and Hox-A11; an siRNA against Hox-A11 partly rescued the differentiation phenotype created by the anti-miR-181 antisense LNA oligonucleotide (FIG. 11e-f). Moreover, differentiation was inversely correlated with Hox-A11 expression (FIG. 11g); Hox-A11 was absent in control cells that differentiated normally, and upregulated in LNA-treated cells that did not differentiate. The siRNA reduced Hox-A11 protein levels in LNA-treated cells, resulting in an intermediate level of Hox-A11 protein, as well as an intermediate level of differentiation. Thus Hox-A11 is a critical target of miR-181. As Hox-A11 is a repressor of myoblast terminal differentiation in vitro²³, our data support a model in which miR-181 participates in differentiation by alleviating repression by Hox-A11, which in turn results in MyoD induction and triggers the expression of muscle markers (FIG. 12). We note, however, that as inhibition of miR-181 did not completely abolish Hox-A11 downregulation (FIG. 11b), additional mechanisms must control cellular Hox-A11 levels during myoblastic differentiation. A plausible hypothesis is that Hox-A11 mRNA translation is controlled by more than one miRNA. Indeed, in the reporter assay described in FIG. 11d, insertion of only one target sequence did not influence reporter activity, and inhibition required multiple targets in the reporter. This is generally the case for miRNAs acting at the translational level, as documented previously^{26,27}, and is consistent with the observation that several miRNAs generally bind to natural target mRNAs. A number of other miRNAs are predicted to bind to the natural Hox-A11 3′ untranslated region (UTR). Among these, several are expressed and/or upregulated in terminally differentiating myoblasts (miR23a, miR188, miR339 and miR30b; FIG. 15c). The coordinate action of these miRNAs may lead to the complete disappearance of Hox-A11 on differentiation.

Our results also suggest that Hox-A11 is not the only target of miR-181. First, restoration of the LNA-induced differentiation phenotype by treatment with anti-Hox-A11 siRNA was only partial (FIG. 11f), perhaps due to the residual level of Hox-A11 protein observed in these cells (FIG. 11g). Second, the involvement of other protein targets in miR-181 pathways would be consistent with the marked phenotype created by downregulating this miRNA. Indeed, miRNAs do seem to have multiple targets, including in mammals²⁸. From in silico analysis, miR-181a is suspected to have a number of targets in addition to Hox-A11, some of which are relevant to skeletal-muscle terminal differentiation, such as ID2 (a bHLH-related inhibitor of differentiation and DNA binding) or EYA1 (a homologue of the Drosophila eyes absent transcription factor; see http://www.microma.org/). As miR-181 is also involved in B lymphocyte differentiation⁹, it will be important to determine whether the pathways in which it participates are common to these two cell types, and are more widespread.

Taken together, our data demonstrate that miR-181 is required for skeletal myoblast terminal differentiation, during which an important target is the homeobox protein Hox-A11. However, neither upregulation of miR- 181, nor downregulation of Hox-A11, triggered terminal differentiation of proliferating myoblasts (FIG. 15d). Thus, miR-181 is necessary, but not sufficient, for differentiation. miR-181 is expressed at very low levels in adult muscle, in contrast to other miRNAs, such as miR-133 or miR-1, which are readily detected in adult muscle¹². A reasonable explanation is that miR-133 and miR-1 are involved in the maintenance of the muscle phenotype, whereas miR-181 is involved in its establishment. Our results underscore the importance of dynamic analysis of miRNAs during cell differentiation.

Methods

DNA constructs. The reporter plasmid containing the miR-181a complementary sequence was constructed by inserting a synthetic double-stranded oligonucleotide into the Sall-Xbal site of pISO vector (kind gift of D. Bartel) downstream of the firefly luciferase coding sequence. Reporter plasmids with four copies of the predicted Hox-A11 target sequence for miR-181 (position of the binding nucleus, 1361 in mRNA, NM_{—}005523), either wild type or mutated, were constructed by cloning four tandem repeats of a 64 base pair sequence containing the target into the Sall-Xbal sites of pISO. Details of construction are available on request. The pISO reporter plasmid containing the miR196 target site from Hox-B8 was a kind gift of D. Bartel.

Oligonucleotides. SiRNA, RNA, and DNA oligonucleotides were obtained from various sources; LNA probes discriminating miR-181 a, b and c isoforms were from Exiquon (Vedbaek, Denmark). LNA-DNA mixed oligonucleotides were from Proligo (Paris, France). The anti-Hox-A11 siRNA sequence was: 5′-GAGCUCGGCCMCGUCUACTT-3′.

Cell culture and transfection. Myoblast C2C12 cells were maintained in Dulbecco's minimal essential medium (DMEM; Gibco-lnvitrogen, Paisley, UK) supplemented with 15% fetal bovine serum (Dominique Dutscher, Brumath, France). For transfections, 5×10⁴ cells were plated in 6-well plates and transfected the next day with 1 μg of oligonucleotides using Lipofectamine (Gibco-lnvitrogen). To induce terminal differentiation, 24 h after transfection, growth medium was replaced by differentiation medium (DMEM, 2% horse serum, Invitrogen). Phenotypic differentiation was observed after 72-96 h. For luciferase assays, HeLa S3 cells were grown in complete DMEM in 24-well plates. Cells were transfected, using Lipofectamine 2000, with firefly luciferase reporter vectors (0.05 μg), together with a Renilla luciferase control vector (0.1 μg) (Promega, Lyon, France) and equal amounts (as assessed by A260 and by gel analysis) of synthetic miRNA duplexes (0.1 μg). Luciferase activity was measured 24 h post transfection and was normalized using Renilla luciferase activity. C2C12 cells were transfected with firefly luciferase reporter constructs containing a sequence complementary to miR-181 in their 3′ UTR, or a mutated version of this sequence, along with a Renilla construct to monitor transfection efficiency, and placed under differentiation conditions; reporter activities were measured after 2 days in differentiation medium.

Mouse embryonic stem cells (kind gift of M. Vernet) were cultured and maintained in an undifferentiated state using standard procedures. For embryoid body formation they were diluted and kept in suspension for 4 days. To induce muscle differentiation, embryoid bodies were maintained an additional 2 days in 1% DMSO in DMEM with 10% horse serum10, then plated on tissue culture dishes in DMEM with 2% horse serum. Myotubes were observed at 21 days of differentiation.

Cardiotoxin-induced regeneration assay. Six- to seven-week-old Balb/c mice were anaesthetized with Avertin (Sigma-Aldrich, Steinheim, Germany), the leg was shaved, and injections were made into the tibialis anterior muscle, using a 29 G ½ insulin syringe. Cardiotoxin (Latoxan, Valence, France) was diluted to 10 μM in PBS, and 25 μl were injected per muscle²⁹. Control animals received equal volumes of PBS. Tibialis anterior muscles were harvested at different times, and total RNA was extracted and used for northern blots, or muscles were frozen as described below for in situ hybridization.

Northern blot. For northern blot analyses, 30 μg of total RNA were separated on 15% denaturing polyacrylamide gels and electro-transferred to Hybond-N+ (Amersham Biosciences, Little Chalfont, UK) membranes. DNA anti-sense probes were end-labelled with T4 polynucleotide kinase (BioLabs, Hitchin, UK) using y-32P-ATP with high specific activity (6,000 Ci mmol-1, Amersham Biosciences). Hybridization was carried out in Rapid-hyb buffer (Amersham Biosciences) at 42° C. Washing was carried out at 42° C. (DNA probes) or at 65° C. (LNA probes). U6 expression was used as a loading control.

In situ hybridization and immuno-FISH. The miRNA in situ hybridization protocol by Ambion (Austin, Tex.) was optimized for skeletal muscle. Briefly, the tibialis anterior muscle was frozen in Tissue-Tek OCT reagent, cryo-sections (12 μm) were prepared, de-proteinized and acetylated as previously described30. Digoxigenin (DIG)-labelled miRNA probes (miR-181: sequence complementary to the mature miRNA) were prepared according to the instructions for the mirVana Probe Construction kit (Ambion). Muscle crosssections were incubated with specific miRNA probes (500 ng/ml) overnight at 38° C., washed with 2. SSC at 38° C. for 20 min, and treated with 400 units/ml (10 μg/ml) RNase A at 37° C. for 30 min. After two washes with PBS, the slides were incubated with FITC-coupled anti-DIG antibody (Roche, Mannheim, Germany) for 4 h at room temperature, washed, rinsed with 200 ng/ml DAPI in PBS, mounted on glass slides with VectaShield (BioValley, Burlingame, Calif.), and analysed by fluorescent microscopy.

For immuno-FISH experiments, the cryo-sections were fixed with 4% phosphate-buffered paraformaldehyde (PFA) for 10 min, treated for 10 min with 1 μg/ml proteinase K, and post-fixed in 4% PFA for 10 more min. In situ hybridization was performed as described above, and then the sections were incubated 4 h to over-night at room temperature with anti-embryonic MHC antibody (F1.652, Developmental Studies Hybridoma Bank, Iowa City, Iowa.), at 2 μg/ml in PBS-1%BSA-5% newborn calf serum. Detection was performed by 1 h incubation with anti-mouse-TRITC anti-body (Sigma-Aldrich), diluted 1:1,000 in blocking solution.

For all FISH and immuno-FISH experiments, 25-30 cross-sections from four independent regenerating tibialis anterior muscles were analysed. Negative controls with secondary antibodies alone were performed in all cases, and gave no detectable staining.

Real-time RT-PCR. Poly-A+ mRNA was isolated with a MagNA pure LC mRNA isolation kit (Roche). One-step real time Q-RT-PCR was carried out with a Hot start LC RNA master SYBR green kit (Roche), using the following primers: Hox-A11, forward 5′-TCTCTAAGGCTCCAGCCTAC-3′, reverse GCTTAACCACGGA-GATCTGA; MCK, forward 5′-CACCATGCCGTTCGGCAACA-3′, reverse 5′-GGTTGTCCACCCCAGTCT-3′; 36B4 (housekeeping gene used for RNA normalization), forward 5′-ATGTGCAGCTGATAAAGACTGG-3′, reverse 5′-AGGCCTTGACCTTTTCAGTMG-3′.

Western blotting and immunofluorescence microscopy. Western blotting and immunostaining were performed using standard procedures. Rabbit anti-MCK was kindly provided by H. Ito, anti-Hox-A11 was a kind gift of L. Patterson. Anti-tubulin, anti-actin, anti-MHC (MY-32) and Cy-3 conjugated anti-mouse IgG were purchased from Sigma, and anti-MyoD antibodies (C20) were purchased from Santa Cruz (Santa Cruz, Calif.).

References

1. Ambros, V. microRNAs: tiny regulators with great potential. Cell 107, 823-826 (2001).
2. Lagos-Quintana, M. et al. Identification of tissue-specific microRNAs from mouse. Curr. Biol. 12, 735-739 (2002).
3. Lagos-Quintana, M. et al. New microRNAs from mouse and human. RNA 9, 175-179 (2003).
4. Olsen, P. H. & Ambros, V. The lin-4 regulatory RNA controls developmental timing in Caenorhabditis elegans by blocking LIN-14 protein synthesis after the initiation of translation. Dev. Biol. 216, 671-680 (1999).
5. Yekta, S., Shih, I. H. & Bartel, D. P. MicroRNA-directed cleavage of HOXB8 mRNA. Science 304, 594-596 (2004).
6. Krichevsky, A. M. et al. A microRNA array reveals extensive regulation of microRNAs during brain development. RNA 9, 1274-1281 (2003).
7. Houbaviy, H. B., Murray, M. F. & Sharp, P. A. Embryonic stem cell-specific microRNAs. Dev. Cell 5, 351-358 (2003).
8. Ambros, V. The functions of animal microRNAs. Nature 431, 350-355 (2004).
9. Chen, C. Z., Li, L., Lodish, H. F. & Bartel, D. P. MicroRNAs modulate hematopoietic lineage differentiation. Science 303, 83-86 (2004).
10. Dinsmore, J. et al. Embryonic stem cells differentiated in vitro as a novel source of cells for transplantation. Cell Transplant. 5, 131-143 (1996).
11. Koshkin, A. A. et al. LNA (Locked Nucleic Acid): An RNA mimic forming exceedingly stable LNA:LNA duplexes. J. Am. Chem. Soc. 120, 13252-13260 (1998).
12. Sempere, L. F. et al. Expression profiling of mammalian microRNAs uncovers a subset of brain-expressed microRNAs with possible roles in murine and human neuronal differentiation. Genome Biol. 5, R13 (2004).
13. Hutvagner, G., Simard, M. J., Mello, C. & Zamore, P. Sequence-specific inhibition of small RNA function. PLOS Biology 2, 1-11 (2004).
14. Meister, G., Landthaler, M., Dorsett, Y. & Tuschl, T. Sequence-specific inhibition of microRNA- and siRNA-induced RNA silencing. RNA 10, 544-550 (2004).
15. Wahlestedt, C. et al. Potent and nontoxic antisense oligonucleotides containing locked nucleic acids. Proc. Natl Acad. Sci. USA 97, 5633-5638 (2000).
16. Fuchtbauer, E. M. & Westphal, H. MyoD and myogenin are coexpressed in regenerating skeletal muscle of the mouse. Dev. Dyn. 193, 34-39 (1992).
17. Lewis, B. P. et al. Prediction of mammalian microRNA targets. Cell 115, 787-798 (2003).
18. Rehmsmeier, M., Steffen, P., Hochsmann, M. & Giegerich, R. Fast and effective prediction of microRNA-target duplexes. RNA 10, 1507-1517 (2004).
19. Krek, A. et al. Combinatorial microRNA target predictions. Nature Genet. 37, 495-500 (2005).
20. Patterson, L. T., Pembaur, M. & Potter, S. S. Hoxa11 and Hoxd11 regulate branching morphogenesis of the ureteric bud in the developing kidney. Development 128, 2153-2161 (2001).
21. Small, K. M. & Potter, S. S. Homeotic transformations and limb defects in Hox A11 mutant mice. Genes Dev. 7, 2318-2328 (1993).
22. Yamamoto, M. et aL Coordinated expression of Hoxa-11 and Hoxa-13 during limb muscle patteming. Development 125,1325-1335 (1998).
23. Yamamoto, M. & Kuroiwa, A. Hoxa-11 and Hoxa-13 are involved in repression of MyoD during limb muscle development. Dev. Growth Differ. 45, 485498 (2003).
24. Takahashi, Y. et al. Expression profiles of 39 HOX genes in normal human adult organs and anaplastic thyroid cancer cell lines by quantitative real-time RT-PCR system. Exp. Cell Res. 293, 144-153 (2004).
25. Poy, M. N. et al. A pancreatic islet-specific microRNA regulates insulin secretion. Nature 432, 226-230 (2004).
26. Doench, J. G., Petersen, C. P. & Sharp, P. A. siRNAs can function as miRNAs. Genes Dev. 17, 438-442 (2003).
27. Pillai, R. S. et al. Inhibition of translational initiation by Let-7 microRNA in human cells. Science 309, 1573-1576 (2005).
28. Lim, L. P. et al. Microarray analysis shows that some microRNAs downregulate large numbers of target mRNAs. Nature 433, 769-773 (2005).
29. Polesskaya, A., Seale, P. & Rudnicki, M. A. Wnt signaling induces the myogenic specification of resident CD45+adult stem cells during muscle regeneration. Cell 113, 841-852 (2003).
30. DeNardi, C. et al. Type 2X-myosin heavy chain is coded by a muscle fibre type-specific and developmentally regulated gene. J. Cell Biol. 123, 823-835 (1993).

### Example 14

### Additional Data

### Efficient Cellular Uptake

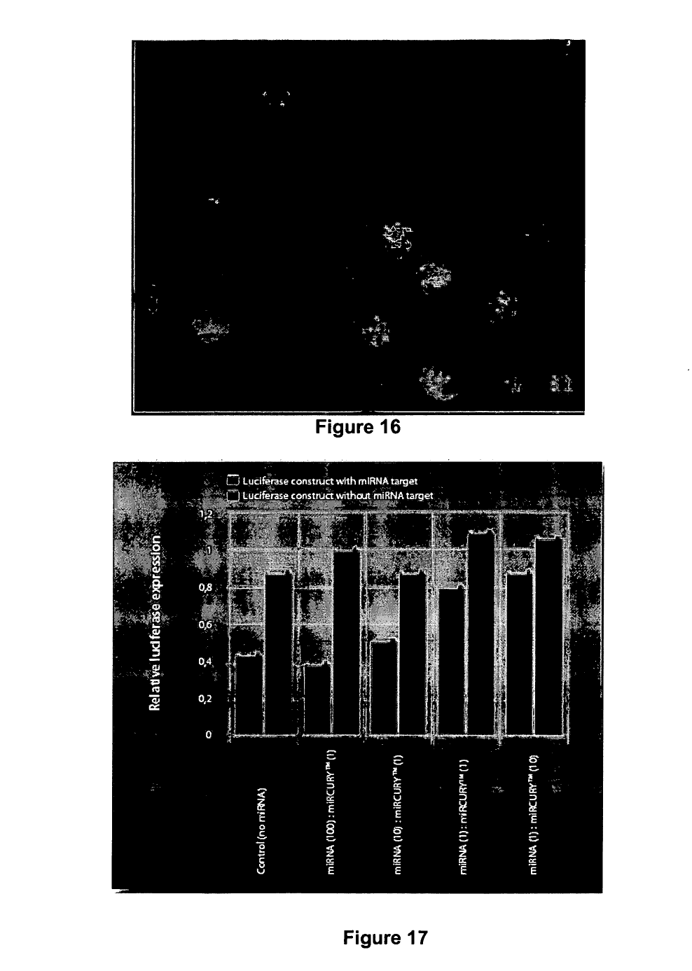

Efficient uptake of fluorochrome-labelled miRCURY™ knockdown probes (LNA probes from Exiqon) into human K562 cells is shown in FIG. 16. The LNA enhanced knockdown antisense molecules are readily transfected into cells by any standard transfection method e.g. electroporation and lipid mediated. The image shows electroporated cells and is kindly provided by Dr. Jens Eriksen, Laboratory of Oncology, Herlev University Hospital, Denmark.

Knockdown of Drosophila bantam microRNA in Transgenic HEK293 Cells

FIG. 17 demonstrates the effective knockdown effect of miRCURY™ antisense molecules. The experimental set-up consists of human HEK 293 cells transfected with a luciferase reporter construct under post-transcriptional control of a miRNA not present in human cells. This reporter system is then co-transfected with synthetic miRNA and the miRCURY™ knockdown antisense in various ratios as indicated. The ratios varies from 100 fold excess of the miRNA showing lack of knock-down effect and up to 10 times excess of the miRCURY™ antisense demonstrating a strong inhibition of the miRNA activity causing up-regulation of the luciferase activity. Data contributed by Dr. Ulf Andersson and Dr. Anders Lund, Biotech Research and Innovation Centre, Copenhagen, Denmark.

Knockdown of Dme-bantam microRNA in Drosophila KC167 Cells

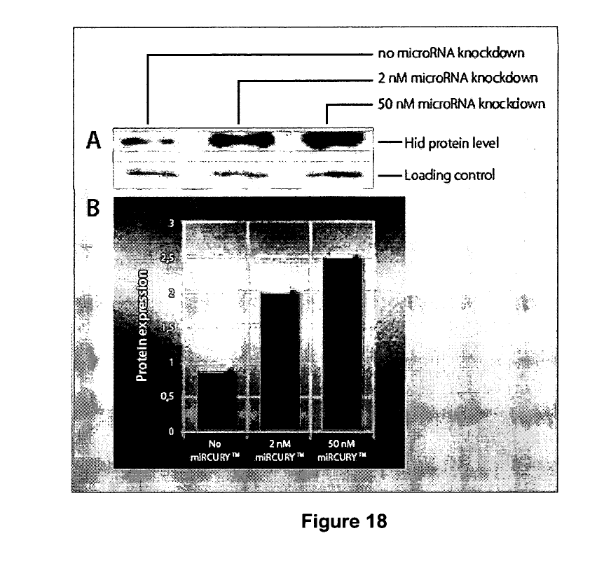

Knockdown of dme-bantam miRNA in Drosophila KC167 cells by miR-CURY™ antisense is shown in FIG. 18. The anti-dme-bantam miRCURY™ knock-down was transfected into D. melanogaster KC167 cells. Sequence-specifi c and concentration-dependent knockdown of bantam miRNA is shown by increased hid protein in miRCURY™ inhibitor-transfected cells as assessed by Western blot analysis. The graph (B) is based on digital analysis of the Western blot (A) of the protein under translational control of the dme-bantam miRNA. Data and images are kindly provided by Dr. Ulf Andersson and Dr. Anders Lund, Biotech Research and Innovation Centre, Copenhagen, Denmark.

Other embodiments are in the claims.

What is claimed is:

1. A nucleotide probe that hybridizes to a miRNA, said probe comprising a plurality of LNA monomers.

2. The probe of claim 1, wherein two of said plurality of LNA monomers are disposed 3 nucleotides apart.

3. The probe of claim 2, wherein only naturally-occurring nucleotides are disposed between said two LNA monomers.

4. The probe of claim 2, wherein each of said plurality of LNA monomers is disposed 3 nucleotides from the closest LNA monomer.

5. The probe of claim 1, wherein two of said plurality of LNA monomers are disposed 4 nucleotides apart.

6. The probe of claim 5, wherein only unmodified nucleotides are disposed between said two LNA monomers.

7. The probe of claim 5, wherein each of said plurality of LNA monomers is disposed 4 nucleotides from the closest LNA monomer.

8. The probe of claim 1, wherein two of said plurality of LNA monomers are disposed adjacent to one another.

9. The probe of claim 8, wherein three of said plurality of LNA monomers are disposed adjacent to one another.

10. The probe of claim 9, wherein four of said plurality of LNA monomers are disposed adjacent to one another.

11. The probe of claim 8, wherein said plurality is disposed at the 3′ or 5′ end.

12. The probe of claim 8, wherein said plurality is disposed so that one of said LNA monomers hybridizes to the center of said miRNA.

13. The probe of claim 1, comprising at most one mismatched base.

14. The probe of claim 1, wherein said probe hybridizes to said miRNA under stringent conditions.

15. The probe of claim 14, wherein said probe hybridizes to said miRNA under high stringency conditions.

16. The probe of claim 1, wherein the melting point of the duplex formed between said probe and said miRNA is at least 1° C. higher than the melting point of the duplex formed between said miRNA and a nucleic acid sequence not comprising a LNA monomer.

17. The probe of claim 16, wherein said nucleic acid sequence does not comprises a modified backbone.

18. The probe of claim 16, wherein the melting point of the duplex formed between said probe and said miRNA is at least 5° C. higher.

19. The probe of claim 1, said probe comprising at least 70% DNA.

20. The probe of claim 1, further comprising a 5′ or 3′ amino group.

21. The probe of claim 1, further comprising a 5′ or 3′ label.

22. The probe of claim 21, wherein said label is fluorescent or radioactive.

23. The probe of claim 22, wherein said label comprises fluorescein.

24. The probe of claim 1, said probe comprising at least 10% LNA units.

25. The probe of claim 1, said probe comprising at most 30% LNA units.

26. The probe of claim 1, wherein said probe is at least 8 nucleotides long and at most 30 nucleotides long.

27. A method of creating a nucleotide duplex, said method comprising the steps of:
(a) providing a miRNA; and
(b) contacting said miRNA with a probe of claim 1 that hybridizes to said miRNA

28. The method of claim 27, wherein said contacting occurs in a cell.

29. The method of claim 27, wherein the duplex that forms is a substrate for RNAse H.

30. The method of claim 27, wherein the duplex that forms is not a substrate for RNAse H.

31. A method of inhibiting the biological activity of a miRNA, said method comprising the steps of:
(a) providing said miRNA; and
(b) contacting said miRNA with a probe of claim 1 that hybridizes to said miRNA, thereby inhibiting the biological activity of said miRNA.

32. The method of claim 31, wherein said contacting occurs in a cell.

33. A method of determining the biological activity of a miRNA, said method comprising the steps of:
(a) providing said miRNA;
(b) contacting said miRNA with a probe of claim 1 that hybridizes to said miRNA; and
(c) assaying said biological activity.

34. The method of claim 33, wherein said contacting occurs in a cell.

35. A kit comprising a probe of claim 1 and packaging and labeling indicative of the miRNA to which said probe hybridizes and conditions under which said hybridization occurs.
